(19) United States
(12) Patent Application Publication
(10) Pub. No.: US 2024/0423914 A1
(43) Pub. Date: Dec. 26, 2024

(54) AMIDE CONTAINING LIPIDS

(71) Applicant: Acuitas Therapeutics, Inc., Vancouver (CA)

(72) Inventor: Jason Samuel Tan, Vancouver (CA)

(21) Appl. No.: 18/744,241

(22) Filed: Jun. 14, 2024

Related U.S. Application Data
(60) Provisional application No. 63/508,782, filed on Jun. 16, 2023.

(51) Int. Cl. A61K 9/127; A61K 9/51; A61K 31/7088; C07C 237/06; C07C 237/10; C07C 271/20; C07D 207/06

(52) U.S. Cl. CPC A61K 9/127; A61K 9/5123; A61K 31/7088; C07C 237/06; C07C 237/10; C07C 271/20; C07D 207/06

(57) ABSTRACT

Compounds are provided having the following Formula (I):

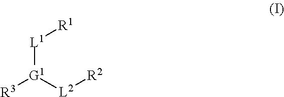

or a pharmaceutically acceptable salt, tautomer, or stereoisomer thereof, wherein G¹, R¹, R², R³, L¹, and L² are as defined herein. Use of the compounds as a component of lipid nanoparticle formulations for delivery of a therapeutic agent, compositions comprising the compounds and methods for their use and preparation are also provided.

### BACKGROUND

### Technical Field

The present disclosure generally relates to novel cationic lipids that can be used in combination with other lipid components, such as neutral lipids, cholesterol, and polymer conjugated lipids, to form lipid nanoparticles with oligonucleotides, to facilitate the intracellular delivery of therapeutic nucleic acids (e.g., oligonucleotides, messenger RNA) both in vitro and in vivo.

### Description of the Related Art

There are many challenges associated with the delivery of nucleic acids to affect a desired response in a biological system. Nucleic acid-based therapeutics have enormous potential but there remains a need for more effective delivery of nucleic acids to appropriate sites within a cell or organism to realize this potential. Therapeutic nucleic acids include, e.g., messenger RNA (mRNA), antisense oligonucleotides, ribozymes, DNAzymes, plasmids, immune stimulating nucleic acids, antagomir, antimir, mimic, supermir, and aptamers. Some nucleic acids, such as mRNA or plasmids, can be used to effect expression of specific cellular products as would be useful in the treatment of, for example, diseases related to a deficiency of a protein or enzyme. The therapeutic applications of translatable nucleotide delivery are extremely broad as constructs can be synthesized to produce any chosen protein sequence, whether indigenous to the system. The expression products of the nucleic acid can augment existing levels of protein, replace missing or non-functional versions of a protein, or introduce a new protein and associated functionality in a cell or organism.

Some nucleic acids, such as miRNA inhibitors, can be used to effect expression of specific cellular products that are regulated by miRNA as would be useful in the treatment of, for example, diseases related to deficiency of protein or enzyme. The therapeutic applications of miRNA inhibition are extremely broad as constructs can be synthesized to inhibit one or more miRNA that would in turn regulate the expression of mRNA products. The inhibition of endogenous miRNA can augment its downstream target endogenous protein expression and restore proper function in a cell or organism as a means to treat disease associated to a specific miRNA or a group of miRNA.

Other nucleic acids can down-regulate intracellular levels of specific mRNA and, as a result, down-regulate the synthesis of the corresponding proteins through processes such as RNA interference (RNAi) or complementary binding of antisense RNA. The therapeutic applications of antisense oligonucleotide and RNAi are also extremely broad, since oligonucleotide constructs can be synthesized with any nucleotide sequence directed against a target mRNA. Targets may include mRNAs from normal cells, mRNAs associated with disease-states, such as cancer, and mRNAs of infectious agents, such as viruses. To date, antisense oligonucleotide constructs have shown the ability to specifically down-regulate target proteins through degradation of the cognate mRNA in both in vitro and in vivo models. In addition, antisense oligonucleotide constructs are currently being evaluated in clinical studies.

However, two problems currently face using oligonucleotides in therapeutic contexts. First, free RNAs are susceptible to nuclease digestion in plasma. Second, free RNAs have limited ability to gain access to the intracellular compartment where the relevant translation machinery resides. Lipid nanoparticles formed from cationic lipids with other lipid components, such as neutral lipids, cholesterol, PEG, PEGylated lipids, and oligonucleotides have been used to block degradation of the RNAs in plasma and facilitate the cellular uptake of the oligonucleotides.

There remains a need for improved cationic lipids and lipid nanoparticles for the delivery of oligonucleotides. Preferably, these lipid nanoparticles would provide optimal drug to lipid ratios, protect the nucleic acid from degradation and clearance in serum, be suitable for systemic delivery, and provide intracellular delivery of the nucleic acid. In addition, these lipid-nucleic acid particles should be well-tolerated and provide an adequate therapeutic index, such that patient treatment at an effective dose of the nucleic acid is not associated with unacceptable toxicity and/or risk to the patient. The present disclosure provides these and related advantages.

### BRIEF SUMMARY

In brief, the present disclosure provides lipid compounds, including stereoisomers, pharmaceutically acceptable salts, or tautomers thereof, which can be used alone or in combination with other lipid components such as neutral lipids, charged lipids, steroids (including for example, all sterols) and/or their analogs, and/or polymer conjugated lipids to form lipid nanoparticles for the delivery of therapeutic agents. In some instances, the lipid nanoparticles are used to deliver nucleic acids such as antisense and/or messenger RNA. Methods for use of such lipid nanoparticles for treatment of various diseases or conditions, such as those caused by infectious entities and/or insufficiency of a protein, are also provided.

In one embodiment, compounds having the following structure of Formula (I):

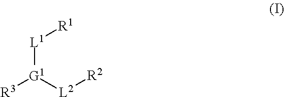

or a pharmaceutically acceptable salt, tautomer, or stereoisomer thereof, wherein G¹, R¹, R², R³, L¹, and L² are as defined herein.

Pharmaceutical compositions comprising one or more of the foregoing compounds of Formula (I) and a therapeutic agent are also provided. In some embodiments, the pharmaceutical compositions further comprise one or more components selected from neutral lipids, charged lipids, steroids, and polymer conjugated lipids. Such compositions are useful for formation of lipid nanoparticles for the delivery of the therapeutic agent.

In other embodiments, the present disclosure provides a method for administering a therapeutic agent to a patient in need thereof, the method comprising preparing a composition of lipid nanoparticles comprising the compound of Formula (I) and a therapeutic agent and delivering the composition to the patient. Such methods are useful for inducing expression of a protein in a subject, for example for expressing an antigen for purposes of vaccination or a gene editing protein.

These and other aspects of the disclosure will be apparent upon reference to the following detailed description.

### DETAILED DESCRIPTION

In the following description, certain specific details are set forth to provide a thorough understanding of various embodiments of the disclosure. However, one skilled in the art will understand that the embodiments of the disclosure may be practiced without these details.

The present disclosure is based, in part, upon the discovery of novel cationic lipids that provide advantages when used in lipid nanoparticles for the in vivo delivery of an active or therapeutic agent such as a nucleic acid into a cell of a mammal. Embodiments of the present disclosure provide nucleic acid-lipid nanoparticle compositions comprising one or more of the novel cationic lipids described herein that provide increased activity of the nucleic acid and improved tolerability of the compositions in vivo, resulting in a significant increase in the therapeutic index as compared to nucleic acid-lipid nanoparticle compositions previously described.

In certain embodiments, the present disclosure provides novel cationic lipids that enable the formulation of improved compositions for the in vitro and in vivo delivery of mRNA and/or other oligonucleotides. In some embodiments, these improved lipid nanoparticle compositions are useful for expression of protein encoded by mRNA. In other embodiments, these improved lipid nanoparticles compositions are useful for upregulation of endogenous protein expression by delivering miRNA inhibitors targeting one specific miRNA or a group of miRNA regulating one target mRNA or several mRNA. In other embodiments, these improved lipid nanoparticle compositions are useful for down-regulating (e.g., silencing) the protein levels and/or mRNA levels of target genes. In some other embodiments, the lipid nanoparticles are also useful for delivery of mRNA and plasmids for expression of transgenes. In yet other embodiments, the lipid nanoparticle compositions are useful for inducing a pharmacological effect resulting from expression of a protein, e.g., increased production of red blood cells through the delivery of a suitable erythropoietin mRNA, or protection against infection through delivery of mRNA encoding for a suitable antibody.

The lipid nanoparticles and compositions of the present disclosure may be used for a variety of purposes, including the delivery of encapsulated or associated (e.g., complexed) therapeutic agents such as nucleic acids to cells, both in vitro and in vivo. Accordingly, embodiments of the present disclosure provide methods of treating or preventing diseases or disorders in a subject in need thereof by contacting the subject with a lipid nanoparticle that encapsulates or is associated with a suitable therapeutic agent, wherein the lipid nanoparticle comprises one or more of the novel cationic lipids described herein.

As described herein, embodiments of the lipid nanoparticles of the present disclosure are particularly useful for the delivery of nucleic acids, including, e.g., mRNA, antisense oligonucleotide, plasmid DNA, microRNA (miRNA), miRNA inhibitors (antagomirs/antimirs), messenger-RNA-interfering complementary RNA (micRNA), DNA, multivalent RNA, dicer substrate RNA, complementary DNA (cDNA), etc. Therefore, the lipid nanoparticles and compositions of the present disclosure may be used to induce expression of a desired protein both in vitro and in vivo by contacting cells with a lipid nanoparticle comprising one or more novel cationic lipids described herein, wherein the lipid nanoparticle encapsulates or is associated with a nucleic acid that is expressed to produce the desired protein (e.g., a messenger RNA or plasmid encoding the desired protein). Alternatively, the lipid nanoparticles and compositions of the present disclosure may be used to decrease the expression of target genes and proteins both in vitro and in vivo by contacting cells with a lipid nanoparticle comprising one or more novel cationic lipids described herein, wherein the lipid nanoparticle encapsulates or is associated with a nucleic acid that reduces target gene expression (e.g., an antisense oligonucleotide or small interfering RNA (siRNA)). The lipid nanoparticles and compositions of the present disclosure may also be used for co-delivery of different nucleic acids (e.g., mRNA and plasmid DNA) separately or in combination, such as may be useful to provide an effect requiring co-localization of different nucleic acids (e.g., mRNA encoding for a suitable gene modifying enzyme and DNA segment(s) for incorporation into the host genome).

Nucleic acids for use with this disclosure may be prepared according to any available technique. For mRNA, the primary methodology of preparation is, but not limited to, enzymatic synthesis (also termed in vitro transcription) which currently represents the most efficient method to produce long sequence-specific mRNA. In vitro transcription describes a process of template-directed synthesis of RNA molecules from an engineered DNA template comprised of an upstream bacteriophage promoter sequence (e.g., including but not limited to that from the T7, T3, and SP6 coliphage) linked to a downstream sequence encoding the gene of interest. Template DNA can be prepared for in vitro transcription from a number of sources with appropriate techniques which are well known in the art including, but not limited to, plasmid DNA and polymerase chain reaction amplification (see Linpinsel, J. L and Conn, G. L., General protocols for preparation of plasmid DNA template and Bowman, J. C., Azizi, B., Lenz, T. K., Ray, P., and Williams, L. D. in RNA in vitro transcription and RNA purification by denaturing PAGE in Recombinant and in vitro RNA syntheses Methods v. 941 Conn G. L. (ed), New York, N.Y. Humana Press, 2012).

Transcription of the RNA occurs in vitro using the linearized DNA template in the presence of the corresponding RNA polymerase and adenosine, guanosine, uridine, and cytidine ribonucleoside triphosphates (rNTPs) under conditions that support polymerase activity while minimizing potential degradation of the resultant mRNA transcripts. In vitro transcription can be performed using a variety of commercially available kits including, but not limited to RiboMax Large Scale RNA Production System (Promega), MegaScript Transcription kits (Life Technologies) as well as with commercially available reagents including RNA polymerases and rNTPs. The methodology for in vitro transcription of mRNA is well known in the art. (see, e.g., Losick, R., 1972, In vitro transcription, Ann Rev Biochem v. 41 409-46; Kamakaka, R. T. and Kraus, W. L. 2001. In Vitro Transcription. Current Protocols in Cell Biology. 2:11.6:11.6.1-11.6.17; Beckert, B. And Masquida, B., (2010) Synthesis of RNA by In Vitro Transcription in RNA in Methods in Molecular Biology v. 703 (Neilson, H. Ed), New York, N.Y. Humana Press, 2010; Brunelle, J. L. and Green, R., 2013, Chapter Five—In vitro transcription from plasmid or PCR-amplified DNA, Methods in Enzymology v. 530, 101-114; all of which are incorporated herein by reference).

The desired in vitro transcribed mRNA is then purified from the undesired components of the transcription or associated reactions (including unincorporated rNTPs, protein enzyme, salts, short RNA oligos etc.). Techniques for the isolation of the mRNA transcripts are well known in the art. Well known procedures include phenol/chloroform extraction or precipitation with either alcohol (ethanol, isopropanol) in the presence of monovalent cations or lithium chloride. Additional, non-limiting examples of purification procedures which can be used include size exclusion chromatography (Lukavsky, P. J. and Puglisi, J. D., 2004, Large-scale preparation and purification of polyacrylamide-free RNA oligonucleotides, RNA v. 10, 889-893), silica-based affinity chromatography and polyacrylamide gel electrophoresis (Bowman, J. C., Azizi, B., Lenz, T. K., Ray, P., and Williams, L. D. in RNA in vitro transcription and RNA purification by denaturing PAGE in Recombinant and in vitro RNA syntheses Methods v. 941 Conn G. L. (ed), New York, N.Y. Humana Press, 2012). Purification can be performed using a variety of commercially available kits including, but not limited to SV Total Isolation System (Promega) and In Vitro Transcription Cleanup and Concentration Kit (Norgen Biotek).

Furthermore, while reverse transcription can yield large quantities of mRNA, the products can contain several aberrant RNA impurities associated with undesired polymerase activity which may need to be removed from the full-length mRNA preparation. These include short RNAs that result from abortive transcription initiation as well as double-stranded RNA (dsRNA) generated by RNA-dependent RNA polymerase activity, RNA-primed transcription from RNA templates and self-complementary 3′ extension. It has been demonstrated that these contaminants with dsRNA structures can lead to undesired immunostimulatory activity through interaction with various innate immune sensors in eukaryotic cells that function to recognize specific nucleic acid structures and induce potent immune responses. This in turn, can dramatically reduce mRNA translation since protein synthesis is reduced during the innate cellular immune response. Therefore, additional techniques to remove these dsRNA contaminants have been developed and are known in the art including but not limited to scalable HPLC purification (see, e.g., Kariko, K., Muramatsu, H., Ludwig, J. And Weissman, D., 2011, Generating the optimal mRNA for therapy: HPLC purification eliminates immune activation and improves translation of nucleoside-modified, protein-encoding mRNA, Nucl Acid Res, v. 39 e142; Weissman, D., Pardi, N., Muramatsu, H., and Kariko, K., HPLC Purification of in vitro transcribed long RNA in Synthetic Messenger RNA and Cell Metabolism Modulation in Methods in Molecular Biology v. 969 (Rabinovich, P. H. Ed), 2013). HPLC purified mRNA has been reported to be translated at much greater levels, particularly in primary cells and in vivo.

A significant variety of modifications have been described in the art which are used to alter specific properties of in vitro transcribed mRNA and improve its utility. These include but are not limited to modifications to the 5′ and 3′ termini of the mRNA. Endogenous eukaryotic mRNA typically contains a cap structure on the 5′-end of a mature molecule which plays an important role in mediating binding of the mRNA Cap Binding Protein (CBP), which is in turn responsible for enhancing mRNA stability in the cell and efficiency of mRNA translation. Therefore, highest levels of protein expression are achieved with capped mRNA transcripts. The 5′-cap contains a 5′-5′-triphosphate linkage between the 5′-most nucleotide and guanine nucleotide. The conjugated guanine nucleotide is methylated at the N7 position. Additional modifications include methylation of the ultimate and penultimate most 5′-nucleotides on the 2′-hydroxyl group.

Multiple distinct cap structures can be used to generate the 5′-cap of in vitro transcribed synthetic mRNA. 5′-capping of synthetic mRNA can be performed co-transcriptionally with chemical cap analogs (i.e., capping during in vitro transcription). For example, the Anti-Reverse Cap Analog (ARCA) cap contains a 5′-5′-triphosphate guanine-guanine linkage where one guanine contains an N7 methyl group as well as a 3′-O-methyl group. However, up to 20% of transcripts remain uncapped during this co-transcriptional process and the synthetic cap analog is not identical to the 5′-cap structure of an authentic cellular mRNA, potentially reducing translatability and cellular stability. Alternatively, synthetic mRNA molecules may also be enzymatically capped post-transcriptionally. These may generate a more authentic 5′-cap structure that more closely mimics, either structurally or functionally, the endogenous 5′-cap which have enhanced binding of cap binding proteins, increased half-life, reduced susceptibility to 5′ endonucleases and/or reduced 5′ decapping. Numerous synthetic 5′-cap analogs have been developed and are known in the art to enhance mRNA stability and translatability (see, e.g., Grudzien-Nogalska, E., Kowalska, J., Su, W., Kuhn, A. N., Slepenkov, S. V., Darynkiewicz, E., Sahin, U., Jemielity, J., and Rhoads, R. E., Synthetic mRNAs with superior translation and stability properties in Synthetic Messenger RNA and Cell Metabolism Modulation in Methods in Molecular Biology v. 969 (Rabinovich, P. H. Ed), 2013).

On the 3′-terminus, a long chain of adenine nucleotides (poly-A tail) is normally added to mRNA molecules during RNA processing. Immediately after transcription, the 3′ end of the transcript is cleaved to free a 3′ hydroxyl to which poly-A polymerase adds a chain of adenine nucleotides to the RNA in a process called polyadenylation. The poly-A tail has been extensively shown to enhance both translational efficiency and stability of mRNA (see Bernstein, P. and Ross, J., 1989, Poly (A), poly (A) binding protein and the regulation of mRNA stability, Trends Bio Sci v. 14 373-377; Guhaniyogi, J. And Brewer, G., 2001, Regulation of mRNA stability in mammalian cells, Gene, v. 265, 11-23; Dreyfus, M. And Regnier, P., 2002, The poly (A) tail of mRNAs: Bodyguard in eukaryotes, scavenger in bacteria, Cell, v. 111, 611-613).

Poly (A) tailing of in vitro transcribed mRNA can be achieved using various approaches including, but not limited to, cloning of a poly (T) tract into the DNA template or by post-transcriptional addition using Poly (A) polymerase. The first case allows in vitro transcription of mRNA with poly (A) tails of defined length, depending on the size of the poly (T) tract, but requires additional manipulation of the template. The latter case involves the enzymatic addition of a poly (A) tail to in vitro transcribed mRNA using poly (A) polymerase which catalyzes the incorporation of adenine residues onto the 3′termini of RNA, requiring no additional manipulation of the DNA template, but results in mRNA with poly(A) tails of heterogenous length. 5′-capping and 3′-poly (A) tailing can be performed using a variety of commercially available kits including, but not limited to Poly (A) Polymerase Tailing kit (EpiCenter), mMESSAGE mMACHINE T7 Ultra kit and Poly (A) Tailing kit (Life Technologies) as well as with commercially available reagents, various ARCA caps, Poly (A) polymerase, etc.

In addition to 5′ cap and 3′ poly adenylation, other modifications of the in vitro transcripts have been reported to provide benefits as related to efficiency of translation and stability. It is well known in the art that pathogenic DNA and RNA can be recognized by a variety of sensors within eukaryotes and trigger potent innate immune responses. The ability to discriminate between pathogenic and self-DNA and RNA has been shown to be based, at least in part, on structure and nucleoside modifications since most nucleic acids from natural sources contain modified nucleosides In contrast, in vitro synthesized RNA lacks these modifications, thus rendering it immunostimulatory which in turn can inhibit effective mRNA translation as outlined above. The introduction of modified nucleosides into in vitro transcribed mRNA can be used to prevent recognition and activation of RNA sensors, thus mitigating this undesired immunostimulatory activity and enhancing translation capacity (see, e.g., Kariko, K. And Weissman, D. 2007, Naturally occurring nucleoside modifications suppress the immunostimulatory activity of RNA: implication for therapeutic RNA development, Curr Opin Drug Discov Devel, v. 10 523-532; Pardi, N., Muramatsu, H., Weissman, D., Kariko, K., In vitro transcription of long RNA containing modified nucleosides in Synthetic Messenger RNA and Cell Metabolism Modulation in Methods in Molecular Biology v. 969 (Rabinovich, P. H. Ed), 2013); Kariko, K., Muramatsu, H., Welsh, F. A., Ludwig, J., Kato, H., Akira, S., Weissman, D., 2008, Incorporation of Pseudouridine Into mRNA Yields Superior Nonimmunogenic Vector With Increased Translational Capacity and Biological Stability, Mol Ther v. 16, 1833-1840. The modified nucleosides and nucleotides used in the synthesis of modified RNAs can be prepared monitored and utilized using general methods and procedures known in the art. A large variety of nucleoside modifications are available that may be incorporated alone or in combination with other modified nucleosides to some extent into the in vitro transcribed mRNA (see, e.g., US Publication No. 2012/0251618). In vitro synthesis of nucleoside-modified mRNA has been reported to have reduced ability to activate immune sensors with a concomitant enhanced translational capacity.

Other components of mRNA which can be modified to provide benefit in terms of translatability and stability include the 5′ and 3′ untranslated regions (UTR). Optimization of the UTRs (favorable 5′ and 3′ UTRs can be obtained from cellular or viral RNAs), either both or independently, have been shown to increase mRNA stability and translational efficiency of in vitro transcribed mRNA (see, e.g., Pardi, N., Muramatsu, H., Weissman, D., Kariko, K., In vitro transcription of long RNA containing modified nucleosides in Synthetic Messenger RNA and Cell Metabolism Modulation in Methods in Molecular Biology v. 969 (Rabinovich, P. H. Ed), 2013).

In addition to mRNA, other nucleic acid payloads may be used for this disclosure. For oligonucleotides, methods of preparation include but are not limited to chemical synthesis and enzymatic, chemical cleavage of a longer precursor, in vitro transcription as described above, etc. Methods of synthesizing DNA and RNA nucleotides are widely used and well known in the art (see, e.g., Gait, M. J. (ed.) Oligonucleotide synthesis: a practical approach, Oxford [Oxfordshire], Washington, D.C.: IRL Press, 1984; and Herdewijn, P. (ed.) Oligonucleotide synthesis: methods and applications, Methods in Molecular Biology, v. 288 (Clifton, N.J.) Totowa, N.J.: Humana Press, 2005; both of which are incorporated herein by reference).

For plasmid DNA, preparation for use with this disclosure commonly utilizes but is not limited to expansion and isolation of the plasmid DNA in vitro in a liquid culture of bacteria containing the plasmid of interest. The presence of a gene in the plasmid of interest that encodes resistance to a particular antibiotic (penicillin, kanamycin, etc.) allows those bacteria containing the plasmid of interest to selective grow in antibiotic-containing cultures. Methods of isolating plasmid DNA are widely used and well known in the art (see, e.g., Heilig, J., Elbing, K. L. and Brent, R (2001) Large-Scale Preparation of Plasmid DNA. Current Protocols in Molecular Biology. 41:II:1.7:1.7.1-1.7.16; Rozkov, A., Larsson, B., Gillström, S., Björnestedt, R. and Schmidt, S. R. (2008), Large-scale production of endotoxin-free plasmids for transient expression in mammalian cell culture. Biotechnol. Bioeng., 99: 557-566; and U.S. Pat. No. 6,197,553B1). Plasmid isolation can be performed using a variety of commercially available kits including, but not limited to Plasmid Plus (Qiagen), GenJET plasmid MaxiPrep (Thermo) and PureYield MaxiPrep (Promega) kits as well as with commercially available reagents.

Various exemplary embodiments of Formula (I), lipid nanoparticles and compositions comprising the same, and their use to deliver active or therapeutic agents such as nucleic acids to modulate gene and protein expression, are described in further detail below.

As used herein, the following terms have the meanings ascribed to them unless specified otherwise.

Unless the context requires otherwise, throughout the present specification and claims, the word “comprise” and variations thereof, such as, “comprises” and “comprising” are to be construed in an open and inclusive sense, that is, as “including, but not limited to”.

Reference throughout this specification to “one embodiment” or “an embodiment” means that a particular feature, structure, or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the features, structures, or characteristics may be combined in any suitable manner in one or more embodiments.

Unless defined otherwise, all technical and scientific terms used herein have the same meaning as is commonly understood by one of skill in the art to which this disclosure belongs. As used in the specification and claims, the singular form “a”, “an” and “the” include plural references unless the context clearly dictates otherwise.

The phrase “induce expression of a desired protein” refers to the ability of a nucleic acid to increase expression of the desired protein. To examine the extent of protein expression, a test sample (e.g., a sample of cells in culture expressing the desired protein) or a test mammal (e.g., a mammal such as a human or an animal model such as a rodent (e.g., mouse) or a non-human primate (e.g., monkey) model) is contacted with a nucleic acid (e.g., nucleic acid in combination with a lipid of the present disclosure). Expression of the desired protein in the test sample or test animal is compared to expression of the desired protein in a control sample (e.g., a sample of cells in culture expressing the desired protein) or a control mammal (e.g., a mammal such as a human or an animal model such as a rodent (e.g., mouse) or non-human primate (e.g., monkey) model) that is not contacted with or administered the nucleic acid. When the desired protein is present in a control sample or a control mammal, the expression of a desired protein in a control sample or a control mammal may be assigned a value of 1.0. In some embodiments, inducing expression of a desired protein is achieved when the ratio of desired protein expression in the test sample or the test mammal to the level of desired protein expression in the control sample or the control mammal is greater than 1, for example, about 1.1, 1.5, 2.0. 5.0 or 10.0. When a desired protein is not present in a control sample or a control mammal, inducing expression of a desired protein is achieved when any measurable level of the desired protein in the test sample or the test mammal is detected. One of ordinary skill in the art will understand appropriate assays to determine the level of protein expression in a sample, for example dot blots, northern blots, in situ hybridization, ELISA, immunoprecipitation, enzyme function, and phenotypic assays, or assays based on reporter proteins that can produce fluorescence or luminescence under appropriate conditions.

The phrase “inhibiting expression of a target gene” refers to the ability of a nucleic acid to silence, reduce, or inhibit the expression of a target gene. To examine the extent of gene silencing, a test sample (e.g., a sample of cells in culture expressing the target gene) or a test mammal (e.g., a mammal such as a human or an animal model such as a rodent (e.g., mouse) or a non-human primate (e.g., monkey) model) is contacted with a nucleic acid that silences, reduces, or inhibits expression of the target gene. Expression of the target gene in the test sample or test animal is compared to expression of the target gene in a control sample (e.g., a sample of cells in culture expressing the target gene) or a control mammal (e.g., a mammal such as a human or an animal model such as a rodent (e.g., mouse) or non-human primate (e.g., monkey) model) that is not contacted with or administered the nucleic acid. The expression of the target gene in a control sample or a control mammal may be assigned a value of 100%. In particular embodiments, silencing, inhibition, or reduction of expression of a target gene is achieved when the level of target gene expression in the test sample or the test mammal relative to the level of target gene expression in the control sample or the control mammal is about 95%, 90%, 85%, 80%, 75%, 70%, 65%, 60%, 55%, 50%, 45%, 40%, 35%, 30%, 25%, 20%, 15%, 10%, 5%, or 0%. In other words, the nucleic acids are capable of silencing, reducing, or inhibiting the expression of a target gene by at least about 5%, 10%, 15%, 20%, 25%, 30%, 35%, 40%, 45%, 50%, 55%, 60%, 65%, 70%, 75%, 80%, 85%, 90%, 95%, or 100% in a test sample or a test mammal relative to the level of target gene expression in a control sample or a control mammal not contacted with or administered the nucleic acid. Suitable assays for determining the level of target gene expression include, without limitation, examination of protein or mRNA levels using techniques known to those of skill in the art, such as, e.g., dot blots, northern blots, in situ hybridization, ELISA, immunoprecipitation, enzyme function, as well as phenotypic assays known to those of skill in the art.

An “effective amount” or “therapeutically effective amount” of an active agent or therapeutic agent such as a therapeutic nucleic acid is an amount sufficient to produce the desired effect, e.g., an increase or inhibition of expression of a target sequence in comparison to the normal expression level detected in the absence of the nucleic acid. An increase in expression of a target sequence is achieved when any measurable level is detected in the case of an expression product that is not present in the absence of the nucleic acid. In the case where the expression product is present at some level prior to contact with the nucleic acid, an in increase in expression is achieved when the fold increase in value obtained with a nucleic acid such as mRNA relative to control is about 1.05, 1.1, 1.2, 1.3, 1.4, 1.5, 1.75, 2, 2.5, 3, 4, 5, 6, 7, 8, 9, 10, 15, 20, 25, 30, 40, 50, 75, 100, 250, 500, 750, 1000, 5000, 10000 or greater. Inhibition of expression of a target gene or target sequence is achieved when the value obtained with a nucleic acid such as antisense oligonucleotide relative to the control is about 95%, 90%, 85%, 80%, 75%, 70%, 65%, 60%, 55%, 50%, 45%, 40%, 35%, 30%, 25%, 20%, 15%, 10%, 5%, or 0%. Suitable assays for measuring expression of a target gene or target sequence include, e.g., examination of protein or RNA levels using techniques known to those of skill in the art such as dot blots, northern blots, in situ hybridization, ELISA, immunoprecipitation, enzyme function, fluorescence or luminescence of suitable reporter proteins, as well as phenotypic assays known to those of skill in the art.

The term “nucleic acid” as used herein refers to a polymer containing at least two deoxyribonucleotides or ribonucleotides in either single- or double-stranded form and includes DNA, RNA, and hybrids thereof. DNA may be in the form of antisense molecules, plasmid DNA, cDNA, PCR products, or vectors. RNA may be in the form of small hairpin RNA (shRNA), messenger RNA (mRNA), antisense RNA, miRNA, micRNA, multivalent RNA, dicer substrate RNA or viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, single nucleotide polymorphisms, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

The term “gene” refers to a nucleic acid (e.g., DNA or RNA) sequence that comprises partial length or entire length coding sequences necessary to produce a polypeptide or precursor polypeptide.

“Gene product,” as used herein, refers to a product of a gene such as an RNA transcript or a polypeptide.

The term “lipid” refers to a group of organic compounds that include, but are not limited to, esters of fatty acids and are generally characterized by being poorly soluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

A “steroid” is a compound comprising the following carbon skeleton:

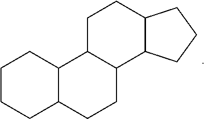 [SMILES: C1CCC2C(C1)CCC1C3CCCC3CCC21]

Non-limiting examples of steroids include cholesterol, and the like.

A “cationic lipid” refers to a lipid capable of being positively charged. Exemplary cationic lipids include one or more amine group(s) which bear the positive charge. Preferred cationic lipids are ionizable such that they can exist in a positively charged or neutral form depending on pH. The ionization of the cationic lipid affects the surface charge of the lipid nanoparticle under different pH conditions. This charge state can influence plasma protein absorption, blood clearance and tissue distribution (Semple, S. C., et al., Adv. Drug Deliv Rev 32:3-17 (1998)) as well as the ability to form endosomolytic non-bilayer structures (Hafez, I. M., et al., Gene Ther 8:1188-1196 (2001)) critical to the intracellular delivery of nucleic acids.

The term “lipid nanoparticle” refers to particles having at least one dimension on the order of nanometers (e.g., 1-1,000 nm) which include one or more of the compounds of Formula (I) or other specified components. In some embodiments, lipid nanoparticles are included in a formulation that can be used to deliver an active agent or therapeutic agent, such as a nucleic acid (e.g., mRNA) to a target site of interest (e.g., cell, tissue, organ, tumor, and the like). In some embodiments, the lipid nanoparticles of the disclosure comprise a nucleic acid. Such lipid nanoparticles typically comprise a compound of Formula (I) and one or more excipient selected from neutral lipids, charged lipids, steroids, and polymer conjugated lipids. In some embodiments, the active agent or therapeutic agent, such as a nucleic acid, may be encapsulated in the lipid portion of the lipid nanoparticle or an aqueous space enveloped by some or all the lipid portion of the lipid nanoparticle, thereby protecting it from enzymatic degradation or other undesirable effects induced by the mechanisms of the host organism or cells, e.g., an adverse immune response.

In various embodiments, the lipid nanoparticles have a mean diameter of from about 30 nm to about 150 nm, from about 40 nm to about 150 nm, from about 40 nm to about 100 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. In certain embodiments, nucleic acids, when present in the lipid nanoparticles, are resistant in aqueous solution to degradation with a nuclease. Lipid nanoparticles comprising nucleic acids and their method of preparation are disclosed in, e.g., U.S. Patent Publication Nos. 2004/0142025, 2007/0042031 and PCT Pub. Nos. WO 2013/016058 and WO 2013/086373, the full disclosures of which are herein incorporated by reference in their entirety for all purposes.

As used herein, “lipid encapsulated” refers to a lipid nanoparticle that provides an active agent or therapeutic agent, such as a nucleic acid (e.g., mRNA), with full encapsulation, partial encapsulation, or both. In an embodiment, the nucleic acid (e.g., mRNA) is fully encapsulated in the lipid nanoparticle.

The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-DMG) and the like.

The term “neutral lipid” refers to any of several lipid species that exist either in an uncharged or neutral zwitterionic form at a selected pH. At physiological pH, such lipids include, but are not limited to, phosphotidylcholines such as 1,2-Distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-Palmitoyl-2-oleoyl-sn-glycero-3-phosphocholine (POPC), 1,2-dioleoyl-sn-glycero-3-phosphocholine (DOPC), phophatidylethanolamines such as 1,2-Dioleoyl-sn-glycero-3-phosphoethanolamine (DOPE), sphingomyelins (SM), ceramides, steroids such as sterols and their derivatives. Neutral lipids may be synthetic or naturally derived.

The term “charged lipid” refers to any of several lipid species that exist in either a positively charged or negatively charged form independent of the pH within a useful physiological range, e.g., pH˜3 to pH˜9. Charged lipids may be synthetic or naturally derived. Examples of charged lipids include phosphatidylserines, phosphatidic acids, phosphatidylglycerols, phosphatidylinositols, sterol hemisuccinates, dialkyl trimethylammonium-propanes, (e.g., DOTAP, DOTMA), dialkyl dimethylaminopropanes, ethyl phosphocholines, dimethylaminoethane carbamoyl sterols (e.g., DC-Chol).

As used herein, the term “aqueous solution” refers to a composition comprising water.

“Serum-stable” in relation to nucleic acid-lipid nanoparticles means that the nucleotide is not significantly degraded after exposure to a serum or nuclease assay that would significantly degrade free DNA or RNA. Suitable assays include, for example, a standard serum assay, a DNAse assay, or an RNAse assay.

“Systemic delivery,” as used herein, refers to delivery of a therapeutic product that can result in a broad exposure of an active agent within an organism. Some techniques of administration can lead to the systemic delivery of certain agents, but not others. Systemic delivery means that a useful, preferably therapeutic, amount of an agent is exposed to most parts of the body. Systemic delivery of lipid nanoparticles can be by any means known in the art including, for example, intravenous, intraarterial, subcutaneous, and intraperitoneal delivery. In some embodiments, systemic delivery of lipid nanoparticles is by intravenous delivery.

“Local delivery,” as used herein, refers to delivery of an active agent directly to a target site within an organism. For example, an agent can be locally delivered by direct injection into a disease site such as a tumor, other target site such as a site of inflammation, or a target organ such as the liver, heart, pancreas, kidney, and the like. Local delivery can also include topical applications or localized injection techniques such as intramuscular, subcutaneous, or intradermal injection. Local delivery does not preclude a systemic pharmacological effect.

“Alkyl” refers to a straight or branched hydrocarbon chain radical consisting solely of carbon and hydrogen atoms that is saturated (i.e., contains no double and/or triple bonds), having from one to twenty-four carbon atoms (C₁-C₂₄ alkyl), one to sixteen carbon atoms (C₁-C₁₆ alkyl), one to twelve carbon atoms (C₁-C₁₂ alkyl), six to twenty-four carbon atoms (C₆-C₂₄ alkyl), one to eight carbon atoms (C₁-C₈ alkyl) or one to six carbon atoms (C₁-C₆ alkyl) and which is attached to the rest of the molecule by a single bond, e.g., methyl, ethyl, n-propyl, 1-methylethyl (iso propyl), n-butyl, n-pentyl, 1,1-dimethylethyl (t-butyl), 3-methylhexyl, 2-methylhexyl, and the like. Unless stated otherwise specifically in the specification, an alkyl group is optionally substituted.

“Alkoxy” refers to a radical with a formula —OR^{a} where R^{a} is an alkyl radical as defined above. Unless stated otherwise specifically in the specification, an alkoxy group is optionally substituted.

“Alkenyl” refers to a straight or branched hydrocarbon chain radical consisting solely of carbon and hydrogen atoms that contains at least one carbon-carbon double, having from one to twenty-four carbon atoms (C₂-C₂₄ alkenyl), one to twelve carbon atoms (C₂-C₁₂ alkenyl), six to twenty-four carbon atoms (C₆-C₂₄ alkenyl), two to sixteen carbon atoms (C₂-C₁₆ alkenyl), four to twelve carbon atoms (C₄-C₁₂ alkenyl), one to eight carbon atoms (C₂-C₈ alkenyl) or one to six carbon atoms (C₂-C₆ alkenyl) and which is attached to the rest of the molecule by a single bond, e.g., ethenyl, n-propenyl, 1-methylethenyl, n-butenyl, n-pentenyl, 1,1-dimethylethenyl, 3-methylhexenyl, 2-methylhexenyl, and the like. Unless stated otherwise specifically in the specification, an alkenyl group is optionally substituted.

“Alkynyl” refers to a straight or branched hydrocarbon chain radical consisting solely of carbon and hydrogen atoms that contains at least one carbon-carbon triple bond, having from one to twenty-four carbon atoms (C₂-C₂₄ alkynyl), one to twelve carbon atoms (C₂-C₁₂ alkynyl), one to eight carbon atoms (C₂-C₈ alkynyl) or one to six carbon atoms (C₂-C₆ alkynyl) and which is attached to the rest of the molecule by a single bond, e.g., ethynyl, n-propynyl, 1-methylethynyl, n-butynyl, n-pentynyl, 1,1-dimethylethynyl, 3-methylhexynyl, 2-methylhexynyl, and the like. Unless stated otherwise specifically in the specification, an alkynyl group is optionally substituted.

“Alkylene” or “alkylene chain” refers to a straight or branched divalent saturated hydrocarbon chain linking the rest of the molecule to a radical group, consisting solely of carbon and hydrogen. In some embodiments, an alkylene chain has from one to twenty-four carbon atoms (C₁-C₂₄ alkylene), one to fifteen carbon atoms (C₁-C₁₅ alkylene), one to twelve carbon atoms (C₁-C₁₂ alkylene), one to eight carbon atoms (C₁-C₅ alkylene), one to six carbon atoms (C₁-C₆ alkylene), four to six carbon atoms (C₄-C₆ alkylene), two to four carbon atoms (C₂-C₄ alkylene), one to two carbon atoms (C₁-C₂ alkylene), e.g., methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain is optionally substituted.

“Alkenylene” or “alkenylene chain” refers to a straight or branched divalent hydrocarbon chain linking the rest of the molecule to a radical group, consisting solely of carbon and hydrogen and which comprises at least one carbon-carbon double bond. In some embodiments, an alkenylene chain has from two to twenty-four carbon atoms (C₂-C₂₄ alkenylene), two to fifteen carbon atoms (C₂-C₁₅ alkenylene), two to twelve carbon atoms (C₂-C₁₂ alkenylene), two to eight carbon atoms (C₂-C₈ alkenylene), two to six carbon atoms (C₂-C₆ alkenylene), four to six carbon atoms (C₄-C₆ alkenylene), two to four carbon atoms (C₂-C₄ alkenylene), e.g., ethenylene, propenylene, n-butenylene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain is optionally substituted.

“Cycloalkyl” or “carbocyclic ring” refers to a stable non aromatic monocyclic or polycyclic hydrocarbon radical consisting solely of carbon and hydrogen atoms, which may include fused or bridged ring systems, having from three to fifteen ring carbon atoms (C₃-C₁₅), from three to ten ring carbon atoms (C₃-C₁₀) or from three to eight ring carbon atoms (C₃-C₈), and which is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl bicyclo[2.2.1]heptanyl, and the like. Unless otherwise stated specifically in the specification, a cycloalkyl group is optionally substituted.

“Aryl” refers to a carbocyclic ring system radical comprising hydrogen, 6 to 18 carbon atoms and at least one aromatic ring. For purposes of this disclosure, the aryl radical is a monocyclic, bicyclic, tricyclic, or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene.

“Arylalkyl” refers to a radical of the formula —R_{b}-R_{c} where R_{b} is an alkylene or alkenylene as defined above and R_{c} is one or more aryl radicals as defined above, for example, benzyl, diphenylmethyl and the like. Unless stated otherwise specifically in the specification, an arylalkyl group is optionally substituted.

“Heterocyclyl” or “heterocyclic ring” refers to a stable 3- to 18-membered non-aromatic ring radical having one to twelve ring carbon atoms (e.g., two to twelve) and from one to six ring heteroatoms selected from the group consisting of nitrogen, oxygen, and sulfur. Unless stated otherwise specifically in the specification, the heterocyclyl radical is a monocyclic, bicyclic, tricyclic, or tetracyclic ring system, which may include fused, spirocyclic (“spiro-heterocyclyl”) and/or bridged ring systems; and the nitrogen, carbon, or sulfur atoms in the heterocyclyl radical is optionally oxidized; the nitrogen atom is optionally quaternized; and the heterocyclyl radical is partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group is optionally substituted.

The term “substituted” used herein means any of the above groups (e.g., alkyl, alkylhydroxyl, alkenyl, alkynyl, alkylene, cycloalkyl, aryl, aralkyl or heterocyclyl) wherein at least one hydrogen atom is replaced by a bond to a non-hydrogen atoms such as, but not limited to: a halogen atom such as F, Cl, Br, and I; oxo groups (═O); hydroxyl groups (—OH); alkoxy groups (—OR^{a}, where R^{a} is C₁-C₁₂ alkyl or cycloalkyl); carboxyl groups (—OC(═O)R^{a} or —C(═O)OR^{a}, where R^{a} is H, C₁-C₁₂ alkyl or cycloalkyl); amine groups (—NR^{a}R^{b}, where R^{a} and R^{b} are each independently H, C₁-C₁₂ alkyl or cycloalkyl); C₁-C₁₂ alkyl groups; and cycloalkyl groups. In some embodiments the substituent is a C₁-C₁₂ alkyl group. In other embodiments, the substituent is a cycloalkyl group. In other embodiments, the substituent is a halo group, such as fluoro. In other embodiments, the substituent is a oxo group. In other embodiments, the substituent is a hydroxyl group. In other embodiments, the substituent is an alkoxy group. In other embodiments, the substituent is a carboxyl group. In other embodiments, the substituent is an amine group.

“Optional” or “optionally” (e.g., optionally substituted) means that the subsequently described event of circumstances may or may not occur, and that the description includes instances where said event or circumstance occurs and instances in which it does not. For example, “optionally substituted alkyl” means that the alkyl radical may or may not be substituted and that the description includes both substituted alkyl radicals and alkyl radicals having no substitution. In some embodiments, “optionally substituted” means a particular radical is substituted with one or more substituents selected from the group consisting of halo (e.g., F, Cl, Br, and I), oxo (═O), hydroxyl (—OH), alkoxy (—OR^{a}, where R^{a} is C₁-C₁₂ alkyl), cycloalkoxy (—OR^{a}, where R^{a} is C₃-C₈ cycloalkyl), carboxyl (—OC(═O)R^{a} or —C(═O)OR^{a}, where R^{a} is H, C₁-C₁₂ alkyl, or C₃-C₈ cycloalkyl), amine (—NR^{a}R^{b}, where R^{a} and R^{b} are each independently H, C₁-C₁₂ alkyl, or C₃-C₈ cycloalkyl), C₁-C₁₂ alkyl, and C₃-C₈ cycloalkyl.

In some embodiments, “optionally substituted” means substituted with one or more halo substituents. In some embodiments, “optionally substituted” means substituted with one or more oxo substituents. In some embodiments, “optionally substituted” means substituted with one or more hydroxyl substituents. In certain embodiments, “optionally substituted” means substituted with one or more alkoxy substituents. In some embodiments, “optionally substituted” means substituted with one or more cycloalkoxy substituents. In certain embodiments, “optionally substituted” means substituted with one or more carboxy substituents. In some embodiments, “optionally substituted” means substituted with one or more amine substituents. In certain embodiments, “optionally substituted” means substituted with one or more C₁-C₁₂ alkyl substituents. In some embodiments, “optionally substituted” means substituted with one or more C₃-C₈ cycloalkyl substituents.

When a functional group is described as “optionally substituted,” and in turn, substituents on the functional group are also “optionally substituted” and so on, for the purposes of this disclosure, such iterations are limited to five, preferably such iterations are limited to two. In some embodiments, such iterations are limited to one. In some embodiments, such iterations are limited to zero.

This disclosure is also meant to encompass all pharmaceutically acceptable compounds of the compound of Formula (I) being isotopically labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁵C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabeled compounds could be useful to help determine or measure the effectiveness of the compounds, by characterizing, for example, the site or mode of action, or binding affinity to pharmacologically important site of action. Certain isotopically labelled compounds of Formula (I), for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection.

Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O, and ¹³N, can be useful in Positron Emission Topography (PET) studies for examining substrate receptor occupancy. Isotopically labeled compounds of Formula (I) can generally be prepared by conventional techniques known to those skilled in the art or by processes analogous to those described in the Preparations and Examples as set out below using an appropriate isotopically labeled reagent in place of the non-labeled reagent previously employed.

This disclosure is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the disclosure includes compounds produced by a process comprising administering a compound of this disclosure to a mammal for a period sufficient to yield a metabolic product thereof. Such products are typically identified by administering a radiolabeled compound of the disclosure in a detectable dose to an animal, such as rat, mouse, guinea pig, monkey, or to human, allowing sufficient time for metabolism to occur, and isolating its conversion products from the urine, blood, or other biological samples.

“Stable compound” and “stable structure” are meant to indicate a compound that is sufficiently robust to survive isolation to a useful degree of purity from a reaction mixture, and formulation into an efficacious therapeutic agent.

“Mammal” includes humans and both domestic animals such as laboratory animals and household pets (e.g., cats, dogs, swine, cattle, sheep, goats, horses, rabbits), and non-domestic animals such as wildlife and the like.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of the compound of the disclosure. As used herein, the term “solvate” refers to an aggregate that comprises one or more molecules of a compound of the disclosure with one or more molecules of solvent. The solvent may be water, in which case the solvate may be a hydrate. Alternatively, the solvent may be an organic solvent. Thus, the compounds of the present disclosure may exist as a hydrate, including a monohydrate, dihydrate, hemihydrate, sesquihydrate, trihydrate, tetrahydrate and the like, as well as the corresponding solvated forms. The compound of the disclosure may be true solvates, while in other cases, the compound of the disclosure may merely retain adventitious water or be a mixture of water plus some adventitious solvent.

A “pharmaceutical composition” refers to a formulation of a compound of the disclosure and a medium generally accepted in the art for the delivery of the biologically active compound to mammals, e.g., humans. Such a medium includes all pharmaceutically acceptable carriers, diluents, or excipients therefor.

“Effective amount” or “therapeutically effective amount” refers to that amount of a compound of the disclosure which, when administered to a mammal, preferably a human, is sufficient to effect treatment in the mammal, preferably a human. The amount of a lipid nanoparticle of the disclosure which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest in a mammal, preferably a human, having the disease or condition of interest, and includes:
(i) preventing the disease or condition from occurring in a mammal, in particular, when such mammal is predisposed to the condition but has not yet been diagnosed as having it;
(ii) inhibiting the disease or condition, i.e., arresting its development;
(iii) relieving the disease or condition, i.e., causing regression of the disease or condition; or
(iv) relieving the symptoms resulting from the disease or condition, i.e., relieving pain without addressing the underlying disease or condition. As used herein, the terms “disease” and “condition” may be used interchangeably or may be different in that the malady or condition may not have a known causative agent (so that etiology has not yet been worked out) and it is therefore not yet recognized as a disease but only as an undesirable condition or syndrome, wherein a more or less specific set of symptoms have been identified by clinicians.

The compounds of the disclosure, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high-pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers,” which refers to two stereoisomers whose molecules are non-superimposable mirror images of one another.

A “tautomer” refers to a proton shift from one atom of a molecule to another atom of the same molecule. The present disclosure includes tautomers of any said compounds.

### Compounds

In an aspect, the disclosure provides novel lipid compounds which can combine with other lipid components such as neutral lipids, charged lipids, steroids, and/or polymer conjugated lipids to form lipid nanoparticles with oligonucleotides. Without wishing to be bound by theory, it is thought that these lipid nanoparticles shield oligonucleotides from degradation in the serum and provide for effective delivery of oligonucleotides to cells in vitro and in vivo.

Accordingly, one embodiment provides a compound having a structure of Formula (I):

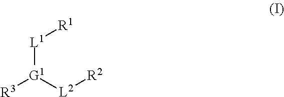

or a pharmaceutically acceptable salt, tautomer, or stereoisomer thereof, wherein:
G¹ is N or CH;
R¹ is —C(═O)N(R^{1a})R^{1b} or —NR^{1c}—C(═O)—R^{1d};
R² is —C(═O)N(R^{2a})R^{2b} or —NR^{2c}—C(═O)—R^{2d};
R^{1c} and R^{2c} are each independently hydrogen, C₁-C₁₆ alkyl, or C₂-C₁₆ alkenyl;
R^{1a} and R^{2a} are each independently C₄-C₂₄ alkyl or C₄-C₂₄ alkenyl;
R^{1b}, R^{2b}, R^{1d}, and R^{2d} is independently C₆-C₂₄ alkyl or C₆-C₂₄ alkenyl;
R³ is —C(═O)—R^{3a} when G¹ is N or R³ is R^{3c}, —NR^{3b}C(═O)—R^{3a} or —OC(═O)—R^{3a} when G¹ is CH;
R^{3a} is C₁-C₈ alkyl or C₁-C₈ alkoxy optionally substituted with one or more substituents selected from the group consisting of halo, oxo, —OH, —CN, —N(R⁴)R⁵, C₁-C₈ alkoxy, C₁-C₈ hydroxylalkoxy, C₁-C₈ alkoxyalkoxy, —C(═O)N(R⁴)R⁵, —NR⁴—C(═O)—R⁵, —N(R⁴)C(═O)OR⁵; —C(═O)OR⁶, —OC(═O)R⁶, cycloalkyl, heterocyclyl, and heteroaryl;
R^{3b} is C₁-C₁₈ alkyl optionally substituted with one or more substituents selected from the group consisting of halo, oxo, —OH, and —N(R⁴)R⁵;
R^{3c} is C₁-C₈ alkoxy optionally substituted with one or more substituents selected from the group consisting of halo, oxo, —OH, —CN, —N(R⁴)R⁵, C₁-C₈ alkoxy, C₁-C₈ hydroxylalkoxy, C₁-C₈ alkoxyalkoxy, —C(═O)N(R⁴)R⁵, —NR⁴—C(═O)—R⁵, —N(R⁴)C(═O)OR⁵; —C(═O)OR⁶, —OC(═O)R⁶, cycloalkyl, heterocyclyl, and heteroaryl;
each occurrence of R⁴ is independently hydrogen or C₁-C₈ alkyl;
each occurrence of R⁵ is independently C₁-C₈ alkyl, C₃-C₈ cycloalkyl, or 3-8 membered heterocyclyl;
R⁶ is hydrogen, C₁-C₁₂ alkyl, C₂-C₁₂ alkenyl, or aralkyl; and
L¹ and L² are each independently C₆-C₁₂ alkylene,
   wherein each alkyl, alkenyl, alkylene, alkoxy, hydroxylalkoxy, alkoxyalkoxy, cycloalkyl, heterocyclyl, heteroaryl and aralkyl is optionally substituted with one or more fluoro.

Another embodiment provides a compound having a structure of Formula (I):

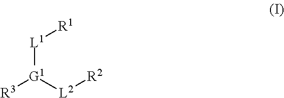

or a pharmaceutically acceptable salt, tautomer, or stereoisomer thereof, wherein:
G¹ is N or CH;
R¹ is —C(═O)N(R^{1a})R^{1b} or —NR^{1c}—C(═O)—R^{1d};
R² is —C(═O)N(R^{2a})R^{2b} or —NR^{2c}—C(═O)—R^{2d};
R^{1c} and R^{2c} are each independently hydrogen, C₁-C₁₆ alkyl, or C₂-C₁₆ alkenyl;
R^{1a} and R^{2a} are each independently C₄-C₂₄ alkyl or C₄-C₂₄ alkenyl;
R^{1b}, R^{2b}, R^{1d}, and R^{2d} is independently C₆-C₂₄ alkyl or C₆-C₂₄ alkenyl;
R³ is —C(═O)—R^{3a} when G¹ is N or R³ is R^{3c}, —NR^{3b}C(═O)—R^{3a} or —OC(═O)—R^{3a} when G¹ is CH;
R^{3a} is C₁-C₈ alkyl, C₃-C₈ cycloalkyl, or C₁-C₈ alkoxy optionally substituted with one or more substituents selected from the group consisting of halo, oxo, —OH, —CN, —N(R⁴)R⁵, C₁-C₈ alkoxy, C₁-C₈ hydroxylalkoxy, C₁-C₈ alkoxyalkoxy, —C(═O)N(R⁴)R⁵, —NR⁴—C(═O)—R⁵, —N(R⁴)C(═O)OR⁵; —C(═O)OR⁶, —OC(═O)R⁶, cycloalkyl, heterocyclyl, and heteroaryl;
R^{3b} is C₁-C₁₈ alkyl optionally substituted with one or more substituents selected from the group consisting of halo, oxo, —OH, and —N(R⁴)R⁵;
R^{3c} is C₁-C₈ alkoxy optionally substituted with one or more substituents selected from the group consisting of halo, oxo, —OH, —CN, —N(R⁴)R⁵, C₁-C₈ alkoxy, C₁-C₈ hydroxylalkoxy, C₁-C₈ alkoxyalkoxy, —C(═O)N(R⁴)R⁵, —NR⁴—C(═O)—R⁵, —N(R⁴)C(═O)OR⁵; —C(═O)OR⁶, —OC(═O)R⁶, cycloalkyl, heterocyclyl, and heteroaryl;
each occurrence of R⁴ is independently hydrogen or C₁-C₈ alkyl;
each occurrence of R⁵ is independently C₁-C₈ alkyl, C₃-C₈ cycloalkyl, or 3-8 membered heterocyclyl;
R⁶ is hydrogen, C₁-C₁₂ alkyl, C₂-C₁₂ alkenyl, or aralkyl;
L¹ is C₅-C₁₂ alkylene; and
L² is C₄-C₁₂ alkylene,
   wherein each alkyl, alkenyl, alkylene, alkoxy, hydroxylalkoxy, alkoxyalkoxy, cycloalkyl, heterocyclyl, heteroaryl and aralkyl is optionally substituted with one or more fluoro.

In some embodiments, the compound has the following Formula (Ia):

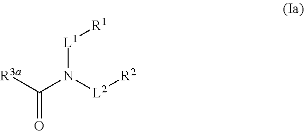

or a pharmaceutically acceptable salt, tautomer, or stereoisomer thereof.

In certain embodiments, the compound has the following Formula (Ib):

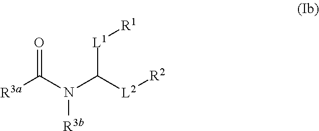

or a pharmaceutically acceptable salt, tautomer, or stereoisomer thereof.

In some embodiments, the compound has the following Formula (Ic):

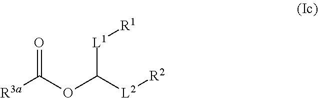

or a pharmaceutically acceptable salt, tautomer, or stereoisomer thereof.

In certain embodiments, the compound has the following Formula (Id):

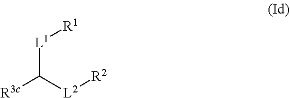

or a pharmaceutically acceptable salt, tautomer, or stereoisomer thereof.

In certain embodiments, R¹ is —C(═O)N(R^{1a})R^{1b}. In some embodiments, R^{1a} is C₄-C₂₄ alkyl.

In certain embodiments, R^{1a} is unbranched and unsubstituted C₆-C₂₄ alkyl. In some embodiments, R^{1a} is unbranched and unsubstituted C₈-C₁₂ alkyl. In certain embodiments, R^{1a} is unbranched and unsubstituted C₁₀ alkyl. In certain embodiments, R^{1a} is unbranched and unsubstituted C₄, C₈, or C₁₀ alkyl.

In some embodiments, R^{1b} is unbranched and unsubstituted C₆-C₂₄ alkyl. In certain embodiments, R^{1b} is unbranched and unsubstituted C₈-C₁₂ alkyl. In some embodiments, R^{1b} is unbranched and unsubstituted C₁₀ alkyl. In certain embodiments, R^{1a} is unbranched and unsubstituted C₄, C₈, or C₁₀ alkyl. In certain embodiments, R^{1b} is branched and unsubstituted C₁₄-C₂₀ alkyl. In some embodiments, R^{1b} is branched and unsubstituted C₁₆-C₁₈ alkyl.

In certain embodiments, R¹ is —NR^{1c}—C(═O)—R^{1d}. In some embodiments, R^{1c} is hydrogen. In certain embodiments, R^{1c} unbranched and unsubstituted C₁-C₁₆ alkyl. In some embodiments, R^{1c} is unbranched and unsubstituted C₁₀ alkyl. In certain embodiments, R^{1c} is unbranched and unsubstituted C₁₂ alkyl. In some embodiments, R^{1c} is unbranched and unsubstituted C₁₀ alkyl or C₁₂ alkyl.

In some embodiments, R^{1d} is unbranched and unsubstituted C₆-C₂₄ alkyl. In certain embodiments, R^{1d} is unbranched and unsubstituted C₈-C₁₂ alkyl. In certain embodiments, R^{1d} is unbranched and unsubstituted C₇-C₁₂ alkyl. In some embodiments, R^{1d} is unbranched and unsubstituted C₉ alkyl. In certain embodiments, R^{1d} is unbranched and unsubstituted C₇ alkyl. In some embodiments, R^{1d} is unbranched and unsubstituted C₇ alkyl or C₉ alkyl.

In certain embodiments, R¹ has one of the following structures:

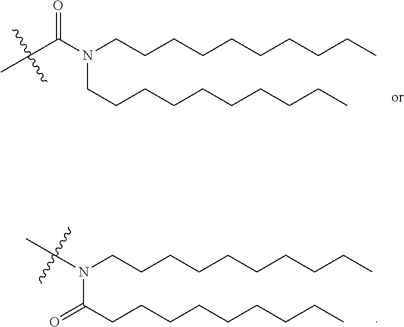 [SMILES: *C(=O)N(CCCCCCCCCC)CCCCCCCCCC ; *N(CCCCCCCCCC)C(=O)CCCCCCCCC]

In some embodiments, R¹ has one of the following structures:

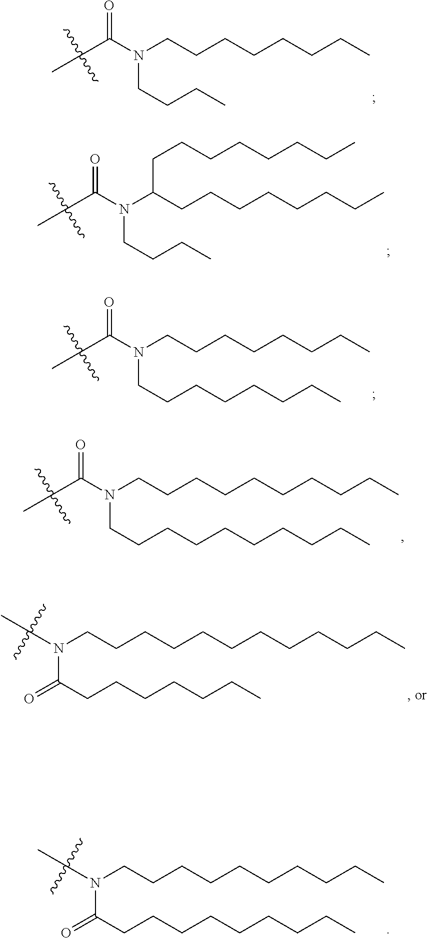

In some embodiments, R² is —C(═O)N(R^{2a})R^{2b}. In certain embodiments, R^{a} is C₄-C₂₄ alkyl. In some embodiments, R^{2a} is unbranched and unsubstituted C₆-C₂₄ alkyl. In certain embodiments, R^{2a} is unbranched and unsubstituted C₈-C₁₂ alkyl. In some embodiments, R¹ is unbranched and unsubstituted C₁₀ alkyl. In some embodiments, R^{a} is unbranched and unsubstituted C₄-C₂₄ alkyl. In certain embodiments, R^{2a} is unbranched and unsubstituted C₄-C₁₂ alkyl. In some embodiments, R^{2a} is unbranched and unsubstituted C₄, C₈, or C₁₀ alkyl.

In certain embodiments, R^{2b} is unbranched and unsubstituted C₆-C₂₄ alkyl. In some embodiments, R^{2b} is unbranched and unsubstituted C₈-C₁₂ alkyl. In certain embodiments, R^{2b} is unbranched and unsubstituted C₁₀ alkyl. In some embodiments, R¹ is unbranched and unsubstituted C₄-C₂₄ alkyl. In certain embodiments, R^{2b} is unbranched and unsubstituted C₄-C₁₂ alkyl. In some embodiments, R^{2b} is unbranched and unsubstituted C₄, C₈, or C₁₀ alkyl.

In some embodiments, R² is —NR^{2c}—C(═O)—R^{2d}. In certain embodiments, R^{2c} is hydrogen. In some embodiments, R^{2c} unbranched and unsubstituted C₁-C₁₆ alkyl. In certain embodiments, R^{2c} is unbranched and unsubstituted C₁₀ alkyl. In some embodiments, R^{2c} is unbranched and unsubstituted C₁₂ alkyl. In certain embodiments, R^{2c} is unbranched and unsubstituted C₁₀ alkyl or C₁₂ alkyl.

In some embodiments, R^{2d} is unbranched and unsubstituted C₆-C₂₄ alkyl. In certain embodiments, R^{2d} is unbranched and unsubstituted C₈-C₁₂ alkyl. In certain embodiments, R^{2d} is unbranched and unsubstituted C₇-C₁₂ alkyl. In some embodiments, R^{2d} is unbranched and unsubstituted C₉ alkyl. In certain embodiments, R^{2d} is unbranched and unsubstituted C₇ alkyl. In some embodiments, R^{2d} is unbranched and unsubstituted C₇ alkyl or C₉ alkyl.

In certain embodiments, R² has one of the following structures:

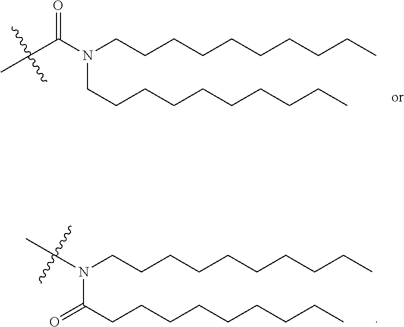 [SMILES: *C(=O)N(CCCCCCCCCC)CCCCCCCCCC ; *N(CCCCCCCCCC)C(=O)CCCCCCCCC]

In some embodiments, R² has one of the following structures:

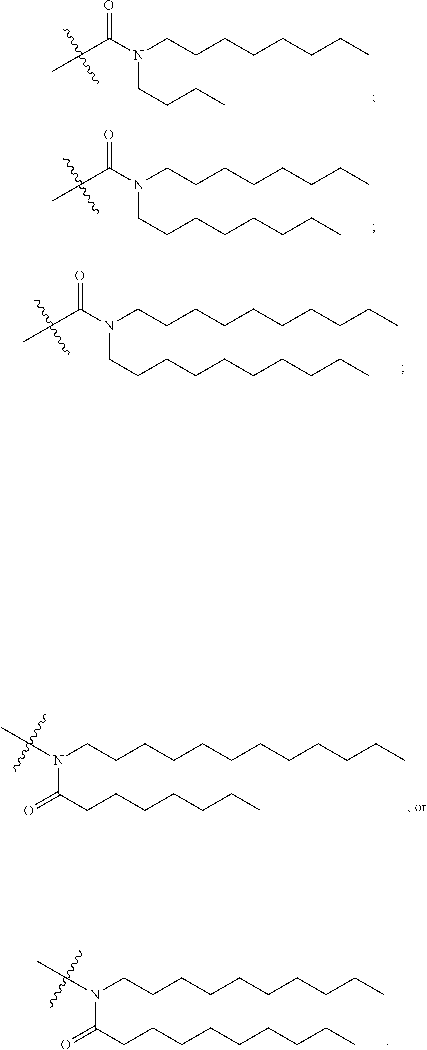 [SMILES: *C(=O)N(CCCC)CCCCCCCC ; *C(=O)N(CCCCCCCC)CCCCCCCC ; *C(=O)N(CCCCCCCCCC)CCCCCCCCCC ; *N(CCCCCCCCCCCC)C(=O)CCCCCCC ; *N(CCCCCCCCCC)C(=O)CCCCCCCCC]

In some embodiments, R^{3a} is C₁-C₈ alkyl optionally substituted with one or more substituents selected from the group consisting of halo (e.g., fluoro), oxo, —OH, —N(CH₃)₂, or 4-6 membered N-heterocyclyl (e.g., pyrrolidinyl, morpholino, piperidinyl, etc.). In certain embodiments, R^{3a} is C₁-C₈ alkoxy optionally substituted with one or more substituents selected from the group consisting of halo (e.g., fluoro), oxo, —OH, —N(CH₃)₂, or 4-6 membered N-heterocyclyl (e.g., pyrrolidinyl, morpholino, piperidinyl, etc.). In some embodiments, R^{3a} is C₁-C₈ alkyl optionally substituted with pyrrolidinyl.

In some embodiments, R^{3a} has one of the following structures:

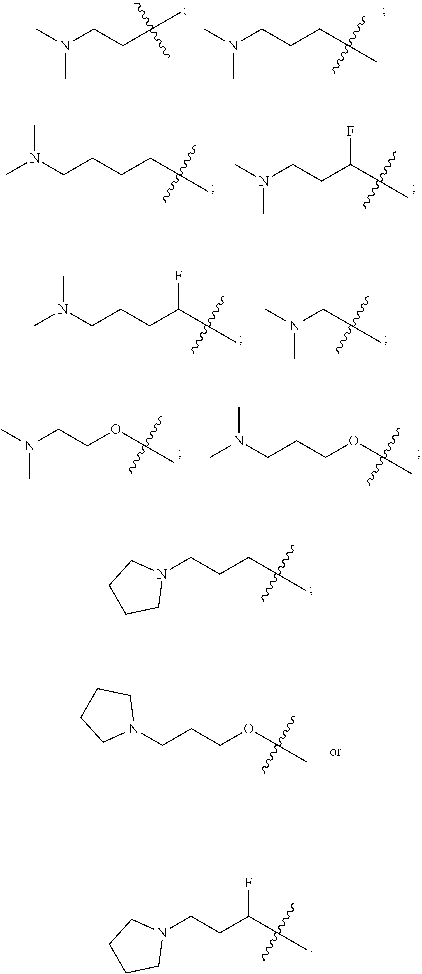

In certain embodiments, R^{3b} is methyl or C₆-C₁₂ alkyl. In some embodiments, R^{3b} is —CH₃ or unbranched and unsubstituted C₈-C₁₀ alkyl.

In some embodiments, R^{3c} is optionally substituted with one or more substituents selected from the group consisting of halo (e.g., fluoro), oxo, —OH, —N(CH₃)₂, or 4-6 membered N-heterocyclyl (e.g., pyrrolidinyl, morpholino, piperidinyl, etc.). In certain embodiments, R^{3c} has one of the following structures:

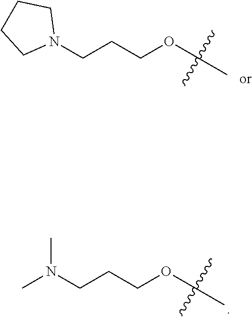

In some embodiments, L¹ is C₅-C₁₀ alkylene and L² is C₄-C₁₀ alkylene. In some embodiments, L¹ is C₅, C₇, C₈, C₉, or C₁₀ alkylene.

In some embodiments, L¹ and L² are each independently C₆-C₁₀ alkylene. In some embodiments, L¹ and L² are each independently C₇, C₈, or C₉ alkylene. In certain embodiments, L¹ and L² are the same. In some embodiments, L¹ and L² are both C₅ alkylene. In certain embodiments, L¹ and L² are both C₇ alkylene. In some embodiments, L¹ and L² are both C₈ alkylene. In certain embodiments, L¹ and L² are both C₉ alkylene. In some embodiments, L¹ and L² are both C₁₀ alkylene.

In certain embodiments, L¹ and L² are different. In some embodiments, L¹ is C₈-C₁₀ alkylene and L² is C₄-C₆ alkylene.

In some embodiments, L¹ and L² are both unbranched and unsubstituted. In some embodiments, L¹ and L² are each independently unbranched and unsubstituted C₆-C₁₀ alkylene. In some embodiments, L¹ and L² are each independently unbranched and unsubstituted C₇, C₈, or C₉ alkylene. In some embodiments, L¹ and L² are both unbranched and unsubstituted C₅ alkylene. In certain embodiments, L¹ and L² are both unbranched and unsubstituted C₇ alkylene. In some embodiments, L¹ and L² are both unbranched and unsubstituted C₈ alkylene. In certain embodiments, L¹ and L² are both unbranched and unsubstituted C₉ alkylene. In some embodiments, L¹ and L² are both unbranched and unsubstituted C₁₀ alkylene.

In certain embodiments, L¹ and L² are different. In some embodiments, L¹ is unbranched and unsubstituted C₈-C₁₀ alkylene and L² is unbranched and unsubstituted C₄-C₆ alkylene. In some embodiments, L¹ is unbranched and unsubstituted C₉ alkylene. In some embodiments, L² is unbranched and unsubstituted C₄ alkylene.

In various embodiments, the compound has one of the structures set forth in Table 1 below (or a stereoisomer, tautomer, or salt thereof).

TABLE 1
Representative compounds of Formula (I)

| No. | Structure |
|---|---|
| I-1 | 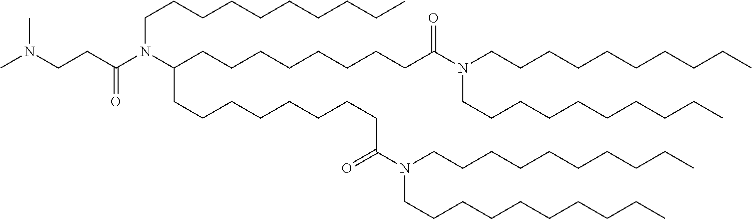 [SMILES: CCCCCCCCCCN(CCCCCCCCCC)C(=O)CCCCCCCCC(CCCCCCCCC(=O)N(CCCCCCCCCC)CCCCCCCCCC)N(CCCCCCCCCC)C(=O)CCN(C)C] |
| I-2 | 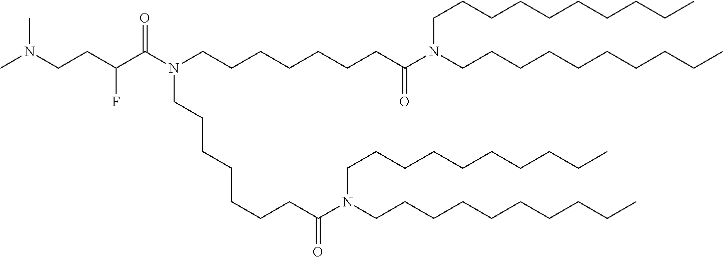 [SMILES: CCCCCCCCCCN(CCCCCCCCCC)C(=O)CCCCCCCN(CCCCCCCC(=O)N(CCCCCCCCCC)CCCCCCCCCC)C(=O)C(F)CCN(C)C] |
| I-3 | 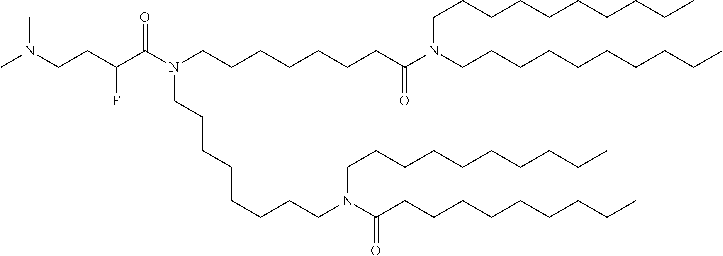 [SMILES: CCCCCCCCCCN(CCCCCCCCN(CCCCCCCC(=O)N(CCCCCCCCCC)CCCCCCCCCC)C(=O)C(F)CCN(C)C)C(=O)CCCCCCCCC] |
| I-4 | 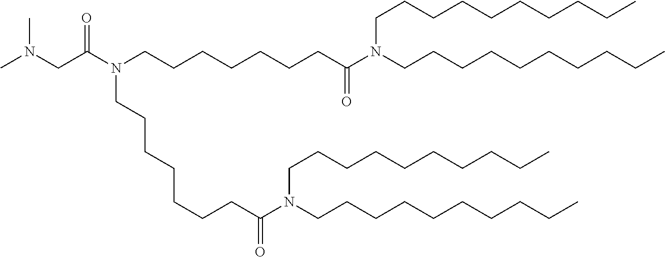 [SMILES: CCCCCCCCCCN(CCCCCCCCCC)C(=O)CCCCCCCN(CCCCCCCC(=O)N(CCCCCCCCCC)CCCCCCCCCC)C(=O)CN(C)C] |
| I-5 | 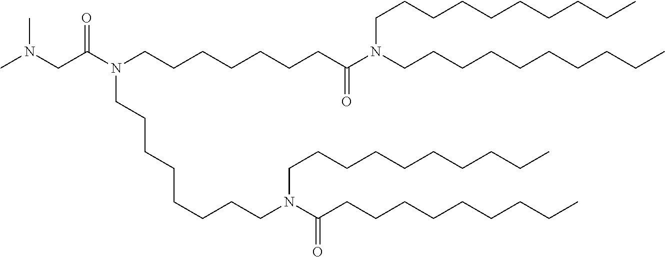 [SMILES: CCCCCCCCCCN(CCCCCCCCN(CCCCCCCC(=O)N(CCCCCCCCCC)CCCCCCCCCC)C(=O)CN(C)C)C(=O)CCCCCCCCC] |
| I-6 | 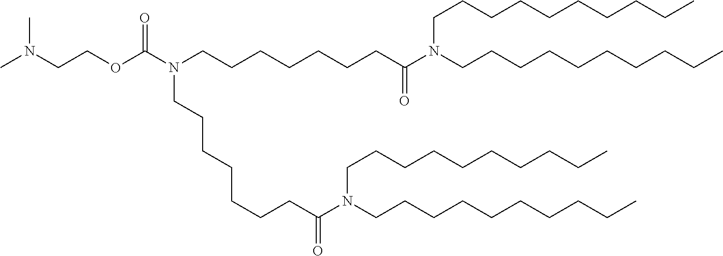 [SMILES: CCCCCCCCCCN(CCCCCCCCCC)C(=O)CCCCCCCN(CCCCCCCC(=O)N(CCCCCCCCCC)CCCCCCCCCC)C(=O)OCCN(C)C] |
| I-7 | 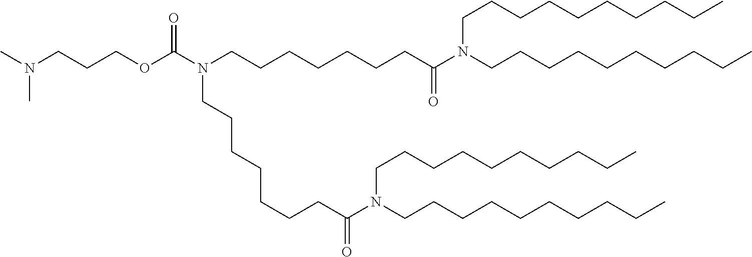 [SMILES: CCCCCCCCCCN(CCCCCCCCCC)C(=O)CCCCCCCN(CCCCCCCC(=O)N(CCCCCCCCCC)CCCCCCCCCC)C(=O)OCCCN(C)C] |
| I-8 | 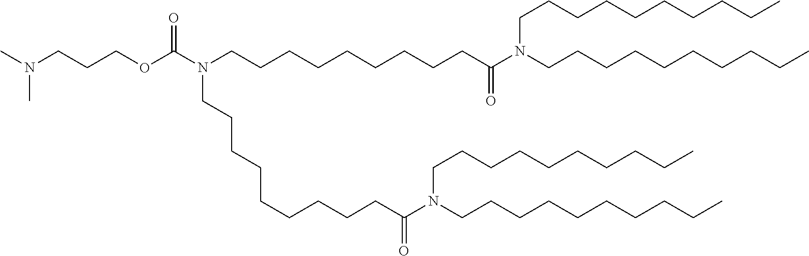 [SMILES: CCCCCCCCCCN(CCCCCCCCCC)C(=O)CCCCCCCCCN(CCCCCCCCCC(=O)N(CCCCCCCCCC)CCCCCCCCCC)C(=O)OCCCN(C)C] |
| I-9 | 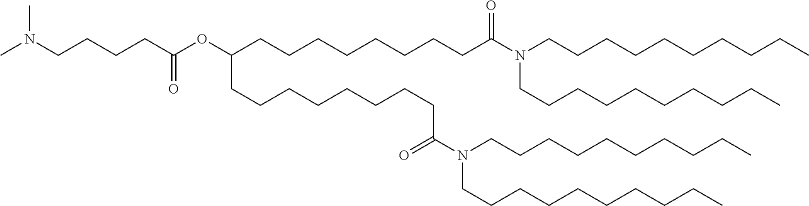 [SMILES: CCCCCCCCCCN(CCCCCCCCCC)C(=O)CCCCCCCCC(CCCCCCCCC(=O)N(CCCCCCCCCC)CCCCCCCCCC)OC(=O)CCCCN(C)C] |
| I-10 | 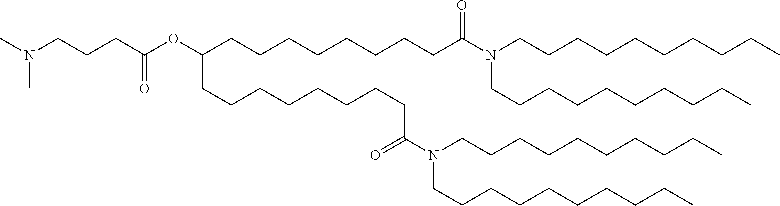 [SMILES: CCCCCCCCCCN(CCCCCCCCCC)C(=O)CCCCCCCCC(CCCCCCCCC(=O)N(CCCCCCCCCC)CCCCCCCCCC)OC(=O)CCCN(C)C] |
| I-11 | 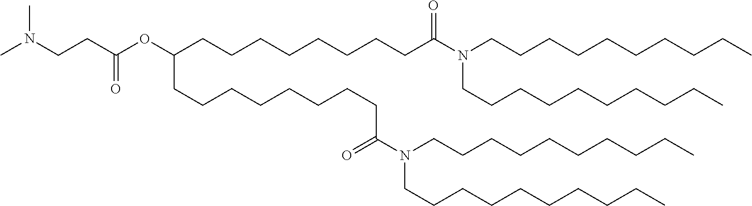 [SMILES: CCCCCCCCCCN(CCCCCCCCCC)C(=O)CCCCCCCCC(CCCCCCCCC(=O)N(CCCCCCCCCC)CCCCCCCCCC)OC(=O)CCN(C)C] |
| I-12 | 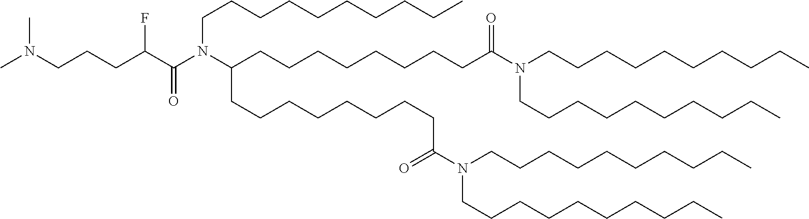 [SMILES: CCCCCCCCCCN(CCCCCCCCCC)C(=O)CCCCCCCCC(CCCCCCCCC(=O)N(CCCCCCCCCC)CCCCCCCCCC)N(CCCCCCCCCC)C(=O)C(F)CCCN(C)C] |
| I-13 | 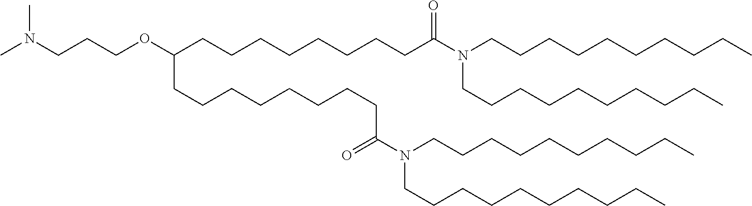 [SMILES: CCCCCCCCCCN(CCCCCCCCCC)C(=O)CCCCCCCCC(CCCCCCCCC(=O)N(CCCCCCCCCC)CCCCCCCCCC)OCCCN(C)C] |
| I-14 | 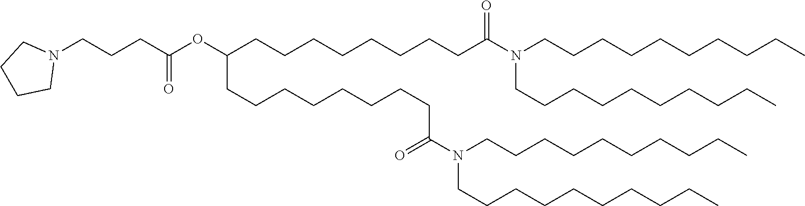 [SMILES: CCCCCCCCCCN(CCCCCCCCCC)C(=O)CCCCCCCCC(CCCCCCCCC(=O)N(CCCCCCCCCC)CCCCCCCCCC)OC(=O)CCCN1CCCC1] |
| I-15 | 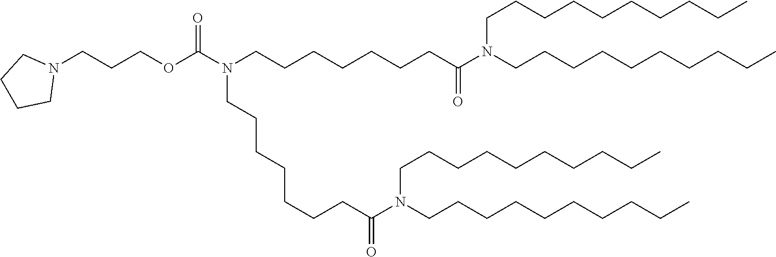 [SMILES: CCCCCCCCCCN(CCCCCCCCCC)C(=O)CCCCCCCN(CCCCCCCC(=O)N(CCCCCCCCCC)CCCCCCCCCC)C(=O)OCCCN1CCCC1] |
| I-16 | 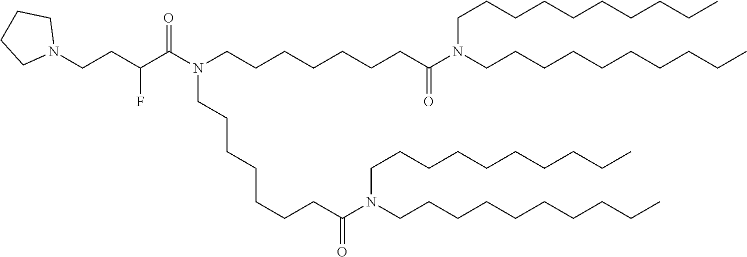 [SMILES: CCCCCCCCCCN(CCCCCCCCCC)C(=O)CCCCCCCN(CCCCCCCC(=O)N(CCCCCCCCCC)CCCCCCCCCC)C(=O)C(F)CCN1CCCC1] |
| I-17 | 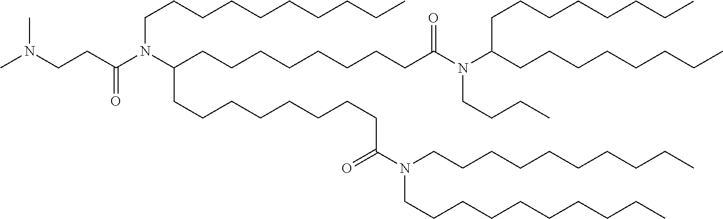 [SMILES: CCCCCCCCCCN(CCCCCCCCCC)C(=O)CCCCCCCCC(CCCCCCCCC(=O)N(CCCC)C(CCCCCCCC)CCCCCCCC)N(CCCCCCCCCC)C(=O)CCN(C)C] |
| I-18 | 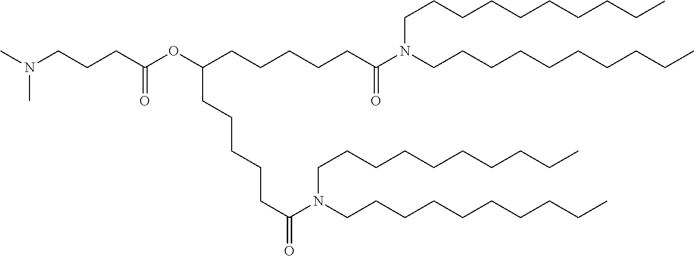 [SMILES: CCCCCCCCCCN(CCCCCCCCCC)C(=O)CCCCCC(CCCCCC(=O)N(CCCCCCCCCC)CCCCCCCCCC)OC(=O)CCCN(C)C] |
| I-19 | 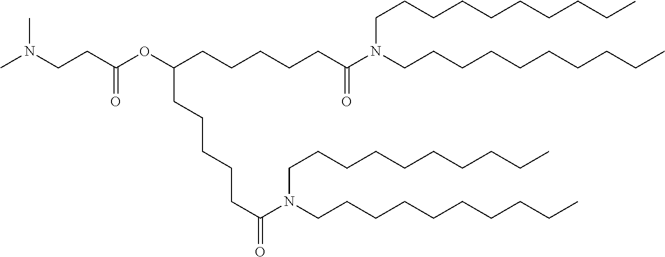 [SMILES: CCCCCCCCCCN(CCCCCCCCCC)C(=O)CCCCCC(CCCCCC(=O)N(CCCCCCCCCC)CCCCCCCCCC)OC(=O)CCN(C)C] |
| I-20 | 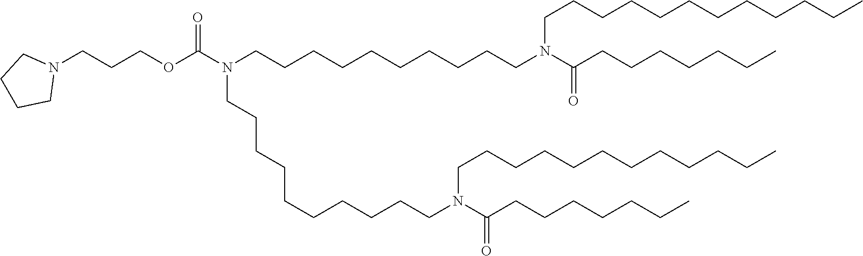 [SMILES: CCCCCCCCCCCCN(CCCCCCCCCCN(CCCCCCCCCCN(CCCCCCCCCCCC)C(=O)CCCCCCC)C(=O)OCCCN1CCCC1)C(=O)CCCCCCC] |
| I-21 | 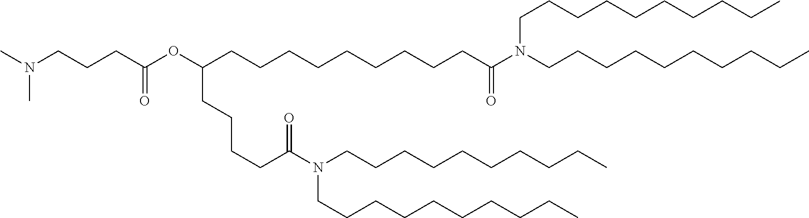 [SMILES: CCCCCCCCCCN(CCCCCCCCCC)C(=O)CCCCCCCCCC(CCCCC(=O)N(CCCCCCCCCC)CCCCCCCCCC)OC(=O)CCCN(C)C] |

It is understood that any embodiment of the compounds of Formula (I), as set forth above, and any specific substituent and/or variable in the compound Formula (I), as set forth above, may be independently combined with other embodiments and/or substituents and/or variables of compounds of Formula (I) to form embodiments of the disclosure not specifically set forth above. In addition, in the event that a list of substituents and/or variables is listed for any particular G group, R group, or L group in a particular embodiment and/or claim, it is understood that each individual substituent and/or variable may be deleted from the particular embodiment and/or claim and that the remaining list of substituents and/or variables will be considered to be within the scope of embodiments of the disclosure.

It is understood that in the present description, combinations of substituents and/or variables of the depicted formulae are permissible only if such contributions result in stable compounds.

For the purposes of administration, the compounds of the present disclosure (typically in the form of lipid nanoparticles in combination with a therapeutic agent) may be administered as a raw chemical or may be formulated as pharmaceutical compositions. Pharmaceutical compositions (e.g., lipid nanoparticles) of the present disclosure comprise a compound of Formula (I) and one or more pharmaceutically acceptable carrier, diluent, or excipient. The compound of Formula (I) is present in the composition in an amount which is effective to form a lipid nanoparticle and deliver the therapeutic agent, e.g., for treating a particular disease or condition of interest. Appropriate concentrations and dosages can be readily determined by one skilled in the art.

An embodiment provides a composition (e.g., lipid nanoparticles) comprising a compound of Formula (I) and a therapeutic agent. In some embodiments, the composition (e.g., lipid nanoparticles) further comprises one or more excipient selected from neutral lipids, steroids, and polymer conjugated lipids.

In some embodiments, the therapeutic agent comprises a nucleic acid. In certain embodiments, the nucleic acid is selected from antisense and messenger RNA.

In certain embodiments, the composition comprises one or more neutral lipids selected from DSPC, DPPC, DMPC, DOPC, POPC, DOPE and SM. In some embodiments, the neutral lipid is DSPC.

In some embodiments, the molar ratio of the compound to the neutral lipid ranges from about 2:1 to about 8:1. In certain embodiments, the steroid is cholesterol. In some embodiments, the molar ratio of the compound to cholesterol ranges from about 2:1 to 1:1. In some embodiments, the ratio is from 5:1 to 1:1 or from 2:1 to 1:1.

In certain embodiments, the polymer conjugated lipid is a pegylated lipid. In various embodiments, the polymer conjugated lipid is a pegylated lipid. For example, some embodiments include a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(m-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω-methoxy(polyethoxy)ethyl)carbamate.

In some embodiments, the molar ratio of the compound to the pegylated lipid ranges from about 100:1 to about 10:1 or from about 100:1 to about 25:1. In some embodiments, the molar ratio of the compound to pegylated lipid ranges from about 100:1 to about 20:1 or from about 100:1 to about 10:1.

In some embodiments, the pegylated lipid is PEG-DMG. In certain embodiments, the pegylated lipid has the following Formula (II):

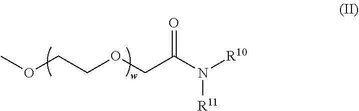

or a pharmaceutically acceptable salt, tautomer, or stereoisomer thereof, wherein:
R¹⁰ and R¹¹ are each independently a straight or branched, alkyl, alkenyl or alkynyl from 10 to 30 carbon atoms, wherein the alkyl, alkenyl or alkynyl is optionally interrupted by one or more ester bonds; and
w has a mean value ranging from 30 to 60.

In certain embodiments, R¹⁰ and R¹¹ are each independently straight alkyl chain containing from 12 to 16 carbon atoms. In some embodiments, the average w is about 45, 46, 47, 48, 49, 50, 51, 52, 53, 54, or 55. In some embodiments, the average w is about 49. In some embodiments, w ranges from 40 to 50. In some embodiments, the average w is about 40 to 50. In certain embodiments, the average w is about 45.

In certain embodiments, the pegylated lipid has the following Formula (II):

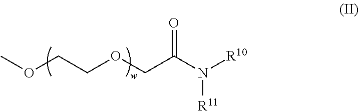

or a pharmaceutically acceptable salt, tautomer, or stereoisomer thereof, wherein:
R¹⁰ and R¹¹ are each independently a straight or branched, alkyl, alkenyl or alkynyl from 10 to 30 carbon atoms, wherein the alkyl, alkenyl or alkynyl is optionally interrupted by one or more ester bonds; and
w has a value ranging from 30 to 60.

In certain embodiments, R¹⁰ and R¹¹ are each independently straight alkyl chain containing from 12 to 16 carbon atoms. In some embodiments, the average w is about 45, 46, 47, 48, 49, 50, 51, 52, 53, 54, or 55. In some embodiments, the average w is about 49. In certain embodiments, w has a value ranging from 30 to 60. In some embodiments, w ranges from 40-50. In some embodiments, w is 40, 41, 42, 43, 44, 45, 46, 47, 48, 49, or 50.

In some embodiments, the lipid nanoparticle or composition comprises a plurality of pegylated lipids of Formula (II) and the average w for the plurality ranges from 40-50. In some embodiments, the average w is 43, 44, 45, 46, 47, or 48.

Synthesis of pegylated lipids can be found in U.S. Pat. No. 9,738,593, the disclosure of which is hereby incorporated by reference.

In some embodiments, the lipid nanoparticle or composition comprises 45-50 mol % of a compound of Formula (I), 5-15 mol % DSPC, 35-45 mol % cholesterol, and 1.5-2.5 mol % based on the total moles of each of the components.

Administration of the compositions of the disclosure can be carried out via any of the accepted modes of administration of agents for serving similar utilities. The pharmaceutical compositions of the disclosure may be formulated into preparations in solid, semi-solid, liquid, or gaseous forms, such as tablets, capsules, powders, granules, ointments, solutions, suspensions, suppositories, injections, inhalants, gels, microspheres, and aerosols. Typical routes of administering such pharmaceutical compositions include, without limitation, oral, topical, transdermal, inhalation, parenteral, sublingual, buccal, rectal, vaginal, and intranasal. The term parenteral as used herein includes subcutaneous injections, intravenous, intramuscular, intradermal, intrasternal injection or infusion techniques. Pharmaceutical compositions of the disclosure are formulated to allow the active ingredients contained therein to be bioavailable upon administration of the composition to a patient. Compositions that will be administered to a subject or patient take the form of one or more dosage units, where for example, a tablet may be a single dosage unit, and a container of a compound of the disclosure in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of a compound of the disclosure, or a pharmaceutically acceptable salt thereof, for treatment of a disease or condition of interest in accordance with the teachings of this disclosure.

A pharmaceutical composition of the disclosure may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example, inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer, or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion, or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer, and isotonic agent may be included.

The liquid pharmaceutical compositions of the disclosure, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose; agents to act as cryoprotectants such as sucrose or trehalose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of the disclosure intended for either parenteral or oral administration should contain an amount of a compound of the disclosure such that a suitable dosage will be obtained.

The pharmaceutical composition of the disclosure may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device.

The pharmaceutical composition of the disclosure may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition of the disclosure may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the disclosure in solid or liquid form may include an agent that binds to the compound of the disclosure and thereby assists in the delivery of the compound. Suitable agents that may act in this capacity include a monoclonal or polyclonal antibody, or a protein.

The pharmaceutical composition of the disclosure may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the disclosure may be delivered in single phase, bi-phasic, or tri-phasic systems to deliver the active ingredient(s). Delivery of the aerosol includes the necessary container, activators, valves, sub-containers, and the like, which together may form a kit. One skilled in the art, without undue experimentation may determine preferred aerosols.

The pharmaceutical compositions of the disclosure may be prepared by methodology well known in the pharmaceutical art. For example, a pharmaceutical composition intended to be administered by injection can be prepared by combining the lipid nanoparticles of the disclosure with sterile, distilled water or other carrier to form a solution. A surfactant may be added to facilitate the formation of a homogeneous solution or suspension. Surfactants are compounds that non-covalently interact with the compound of the disclosure to facilitate dissolution or homogeneous suspension of the compound in the aqueous delivery system.

The compositions of the disclosure, or their pharmaceutically acceptable salts, are administered in a therapeutically effective amount, which will vary depending upon a variety of factors including the activity of the specific therapeutic agent employed; the metabolic stability and length of action of the therapeutic agent; the age, body weight, general health, sex, and diet of the patient; the mode and time of administration; the rate of excretion; the drug combination; the severity of the particular disorder or condition; and the subject undergoing therapy.

Compositions of the disclosure may also be administered simultaneously with, prior to, or after administration of one or more other therapeutic agents. Such combination therapy includes administration of a single pharmaceutical dosage formulation of a composition of the disclosure and one or more additional active agents, as well as administration of the composition of the disclosure and each active agent in its own separate pharmaceutical dosage formulation. For example, a composition of the disclosure and the other active agent can be administered to the patient together in a single oral dosage composition such as a tablet or capsule, or each agent administered in separate oral dosage formulations. Where separate dosage formulations are used, the compounds of the disclosure and one or more additional active agents can be administered at essentially the same time, i.e., concurrently, or at separately staggered times, i.e., sequentially; combination therapy is understood to include all these regimens.

Preparation methods for the above compounds and compositions are described herein below and/or known in the art.

It will be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T.W. and P.G.M. Wutz, Protective Groups in Organic Synthesis (1999), 3^{rd} Ed., Wiley. As one of skill in the art would appreciate, the protecting group may also be a polymer resin such as a Wang resin, Rink resin or a 2-chlorotrityl-chloride resin.

It will also be appreciated by those skilled in the art, although such protected derivatives of compounds of this disclosure may not possess pharmacological activity as such, they may be administered to a mammal and thereafter metabolized in the body to form compounds of the disclosure which are pharmacologically active. Such derivatives may therefore be described as “prodrugs.” All prodrugs of compounds of this disclosure are included within the scope of the disclosure.

Furthermore, all compounds of the disclosure which exist in free base or acid form can be converted to their pharmaceutically acceptable salts by treatment with the appropriate inorganic or organic base or acid by methods known to one skilled in the art. Salts of the compounds of the disclosure can be converted to their free base or acid form by standard techniques.

The following Reaction Scheme illustrates methods to make compounds of this disclosure, i.e., compounds of Formula (I):

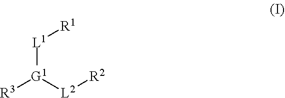

or a pharmaceutically acceptable salt, tautomer, or stereoisomer thereof, wherein G¹, R¹, R², R³, L¹, and L² are as defined herein. It is understood that one skilled in the art may be able to make these compounds by similar methods or by combining other methods known to one skilled in the art. It is also understood that one skilled in the art would be able to make, in a similar manner as described below, other compounds of Formula (I) not specifically illustrated below by using the appropriate starting components and modifying the parameters of the synthesis as needed. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, e.g., Advanced Organic Chemistry: Reactions, Mechanisms, and Structure, 5th edition (Wiley, December 2000)) or prepared as described in this disclosure. Purification (e.g., silica gel chromatography, filtration, extraction, etc.) after each described reaction step is performed as needed.

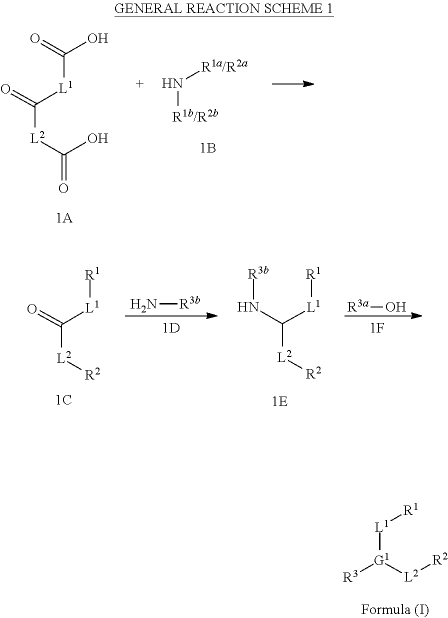

Embodiments of the compound of Formula (I) (e.g., Compound I-1) can be prepared according to General Reaction Scheme 1, wherein the variables (e.g., R¹, R², R³, G¹, L¹, L², R^{1a}, R^{2a}, R^{1b}, R^{2b}, R^{3a}, and R^{3b}) are as defined herein.

Referring to General Reaction Scheme 1, starting materials and other reagents (e.g., compounds 1A, 1B, 1C, and 1F) can be purchased from commercial sources or prepared according to methods familiar to one of ordinary skill in the art. As a first step, a mixture of 1A and 1B is combined under suitable reaction conditions to facilitate a coupling reaction (e.g., DIPEA, HATU in DCM) and afford the desired product as shown. Compound 1C is then reacted with compound 1D using appropriate conditions (e.g., NaBH(AcO)₃, acetic acid and DCE) to give compound 1E. Compound 1E and compound 1F are then combined under appropriate conditions (e.g., DIPEA, HATU in DCM) to produce a compound of Formula (I).

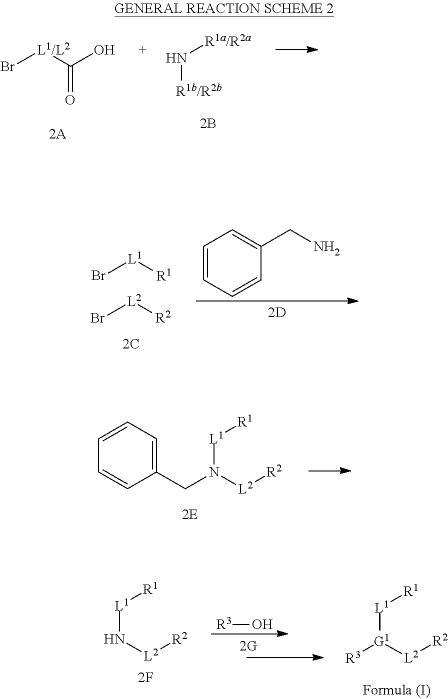

Embodiments of the compound of Formula (I) (e.g., Compound I-2) can be prepared according to General Reaction Scheme 2, wherein the variables (e.g., R¹, R², R³, G¹, L¹, L², R^{1a}, R^{2a}, R^{1b}, R^{2b}, and R^{3b}) are as defined herein.

Referring to General Reaction Scheme 2, starting materials and other reagents (e.g., compounds 2A, 2B, 2D, and 2G) can be purchased from commercial sources or prepared according to methods familiar to one of ordinary skill in the art. As a first step, a mixture of 2A and 2B is combined under suitable reaction conditions to facilitate a coupling reaction (e.g., oxalyl chloride, DMF) and afford the desired product as shown. Compound 2C is then reacted with compound 2D using appropriate conditions (e.g., DIPEA, potassium iodide in acetonitrile) to give compound 2E. Compound 2E is then deprotected using suitable conditions (e.g., H₂, Pd/C in ethyl acetate and methanol) to afford compound 2F. Compound 2F is then reacted with compound 2G (optionally in a protected form) using desired conditions (e.g., DIPEA, HATU in DCM). Optionally, an amine group of R³ may then be deprotected and alkylated under suitable conditions (e.g., (1) TFA in DCM, (2) H₂CO, NaBH(OAc)₃ and methanol). The resultant product is a compound of Formula (I).

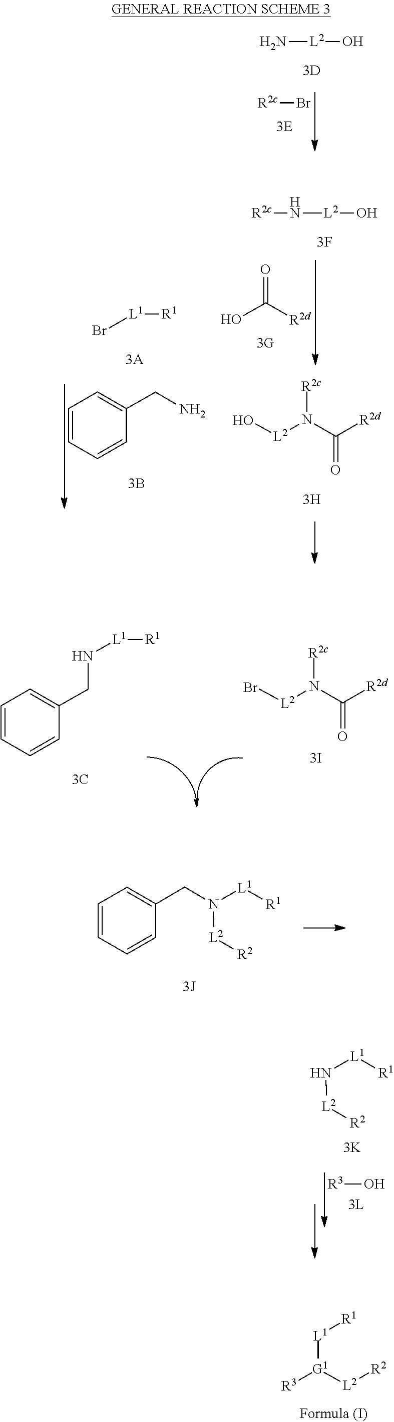

Embodiments of the compound of Formula (I) (e.g., Compound I-3) can be prepared according to General Reaction Scheme 3, wherein the variables (e.g., R¹, R², R³, G¹, L¹, L², R^{2c}, and R^{2d}) are as defined herein.

Referring to General Reaction Scheme 3, starting materials and other reagents (e.g., compounds 3A, 3B, 3D, 3E, and 3G) can be purchased from commercial sources or prepared according to methods familiar to one of ordinary skill in the art. As a first step, a mixture of 3A and 3B is combined under suitable reaction conditions to facilitate a coupling reaction (e.g., DIPEA, potassium iodide in acetonitrile) and afford the desired product as shown.

In parallel, compound 3I is prepared. As a first step, compound 3E and 3D are combined under suitable conditions (e.g., DIPEA in acetonitrile). The resultant compound 3F is then reacted with compound 3G under appropriate conditions (e.g., DIPEA, HATU in DCM) to afford compound 3H. Compound 3H is then converted to compound 3I using suitable conditions (e.g., PBr₃ in diethyl ether). Then, compounds 3C and 3I are combined under appropriate conditions (e.g., DIPEA, potassium iodide in acetonitrile) to afford compound 3J. After a deprotection step (e.g., H₂, Pd/C in ethyl acetate and methanol) to afford compound 3K, a reaction with compounds 3K and 3L (optionally in a protected form) is carried out using suitable conditions (e.g., DIPEA, HATU in DCM). Optionally, an amine group of R³ may then be deprotected and alkylated under suitable conditions (e.g., (1) TFA in DCM, (2) H₂CO, NaBH(OAc)₃ and methanol). The resultant product is a compound of Formula (I).

Alternatively, R³ can be installed with an alkylated amine group by reacting compound 1F, 2F, or 3K under suitable conditions (e.g., dimethylglycinoyl chloride hydrochloride, DMAP, Et₃N in DCM).

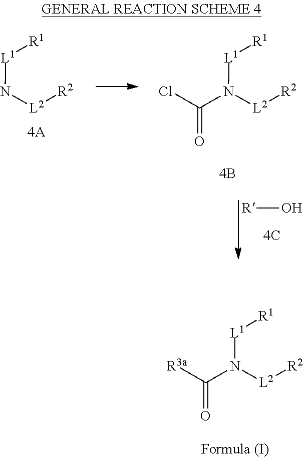

Embodiments of the compound of Formula (I) (e.g., Compound I-7) can be prepared according to General Reaction Scheme 4, wherein the variables (e.g., R¹, R², R³, L¹, and L²) are as defined herein. R′ is a group that, with the oxygen to which it is attached, forms R^{3a} following the coupling reaction as shown.

Referring to General Reaction Scheme 4, starting materials and other reagents (e.g., compound 4A) can be purchased from commercial sources or prepared according to methods familiar to one of ordinary skill in the art or disclosed herein. As a first step, compound 4A is converted to compound 4B under suitable reaction conditions (e.g., triphosgene, sodium bicarbonate in DCM) and afford the desired product 4B as shown. Compound 4B can then be combined with compound 4C under suitable conditions (e.g., heating) to afford a compound of Formula (I) as shown.

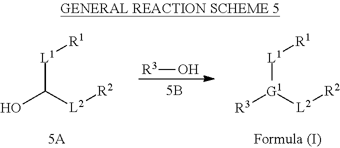

Embodiments of the compound of Formula (I) (e.g., Compound I-9) can be prepared according to General Reaction Scheme 5, wherein the variables (e.g., R¹, R², R³, G¹, L¹, and L²) are as defined herein.

Referring to General Reaction Scheme 5, starting materials and other reagents (e.g., compound 5A and 5B) can be purchased from commercial sources or prepared according to methods familiar to one of ordinary skill in the art or disclosed herein. Compound 5A is reacted with compound 5B under suitable reaction conditions (e.g., DCC, DMAP, Et₃N in DCM) to afford a compound of Formula (I) as shown.

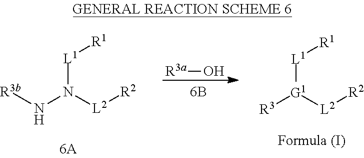

Embodiments of the compound of Formula (I) (e.g., Compound I-12) can be prepared according to General Reaction Scheme 6, wherein the variables (e.g., R¹, R², R³, R^{3b}, R^{3a}, G¹, L¹, and L²) are as defined herein.

Referring to General Reaction Scheme 6, starting materials and other reagents (e.g., compound 6A and 6B) can be purchased from commercial sources or prepared according to methods familiar to one of ordinary skill in the art or disclosed herein. Compound 6A is reacted with compound 6B under suitable reaction conditions (e.g., DIPEA, HATU in DCM) to afford a compound of Formula (I) as shown.

Optionally, if an amine group of R³ is installed in a protected form, it may then be deprotected and alkylated under suitable conditions (e.g., (1) TFA in DCM, (2) H₂CO, NaBH(OAc)₃ and methanol). The resultant product is also a compound of Formula (I).

### Example 1

### Luciferase mRNA In Vivo Evaluation Using

### Lipid Nanoparticle Compositions

A lipid of Formula (I), DSPC, cholesterol and PEG-lipid of Formula (II) are solubilized in ethanol at a molar ratio of 50:10:38.5:1.5 or 47.5:10:40.7:1.8. Lipid nanoparticles (LNP) are prepared at a total lipid to mRNA weight ratio of approximately 10:1 to 40:1. Briefly, the mRNA is diluted to 0.2 mg/mL in 10 to 50 mM citrate buffer, pH 4 to 6 or 10 to 25 mM acetate buffer, pH 4 to 6. Syringe pumps are used to mix the ethanolic lipid solution with the mRNA aqueous solution at a ratio of about 1:5 to 1:3 (vol/vol) with total flow rates above 15 mL/min. The ethanol is then removed, and the external buffer replaced with PBS by dialysis. Finally, the lipid nanoparticles are filtered through a 0.2 μm pore sterile filter.

Studies are performed in 6-8-week-old female C57BU/6 mice (Charles River) or 8-10-week-old CD-1 mice (Charles River or Inotiv) according to guidelines established by an institutional animal care committee (ACC) and the Canadian Council on Animal Care (CCAC). Varying doses of mRNA-lipid nanoparticle are systemically administered by tail vein injection and animals euthanized at a specific time point (e.g., 4 hours) post-administration. Liver and spleen are collected in pre-weighed tubes, weights determined, immediately snap frozen in liquid nitrogen, and stored at −80° C. until processing for analysis.

For liver, approximately 50 mg is dissected for analyses in a 2 mL FastPrep tubes (MP Biomedicals, Solon OH). ¼″ ceramic sphere (MP Biomedicals) is added to each tube and 500-750 μL of Glo Lysis Buffer—GLB (Promega, Madison WI) equilibrated to room temperature is added to liver tissue. Liver tissues are homogenized with the FastPrep24 instrument (MP Biomedicals) at 2×6.0 m/s for 15 seconds. Homogenate is incubated at room temperature for 5 minutes prior to a 1:4 to 1:6 dilution in GLB and assessed using SteadyGlo Luciferase assay system (Promega). Specifically, 50 μL of diluted tissue homogenate is reacted with 50 μL of SteadyGlo substrate, shaken for 10 seconds followed by 5-minute incubation and then luminescence was quantitated using a Filter Max F5 Microplate Reader (Molecular Devices, USA). The amount of protein assayed is determined by using the BCA protein assay kit (Pierce, Rockford, IL). Relative luminescence units (RLU) were then normalized to total μg protein or weight (g) of tissue assayed. To convert RLU to ng luciferase a standard curve is generated with QuantiLum Recombinant Luciferase (Promega).

The FLuc mRNA (7202) from Trilink Biotechnologies will express a luciferase protein, originally isolated from the firefly, Photinus pyralis. FLuc is commonly used in mammalian cell culture to measure both gene expression and cell viability. It emits bioluminescence in the presence of the substrate, luciferin. This capped and polyadenylated mRNA modified with 5-methyoxyuridine and optimized for mammalian systems.

### Example 2

### Immunoglobulin G (IgG) mRNA In Vivo Evaluation Using

### Lipid Nanoparticle Compositions

A lipid of Formula (I), DSPC, cholesterol and PEG-lipid of Formula (II) are solubilized in ethanol at a molar ratio of 50:10:38.5:1.5 or 47.5:10:40.7:1.8. Lipid nanoparticles (LNP) are prepared at a total lipid to mRNA weight ratio of approximately 10:1 to 40:1. Briefly, the mRNA is diluted to 0.2 mg/mL in 10 to 50 mM citrate buffer, pH 4 to 6 or 10 to 25 mM acetate buffer, pH 4 to 6. Syringe pumps are used to mix the ethanolic lipid solution with the mRNA aqueous solution at a ratio of about 1:5 to 1:3 (vol/vol) with total flow rates above 15 mL/min. The ethanol is then removed, and the external buffer replaced with PBS by dialysis. Finally, the lipid nanoparticles are filtered through a 0.2 μm pore sterile filter.

Studies are performed in 6-8-week-old CD-1/ICR mice (Charles River or Inotiv) according to guidelines established by an institutional animal care committee (ACC) and the Canadian Council on Animal Care (CCAC). Varying doses of mRNA-lipid nanoparticle are systemically administered by tail vein injection and animals euthanized at a specific time point (e.g., 24 hours) post-administration. The whole blood is collected, and the serum subsequentially separated by centrifuging the tubes of the whole blood at 2000×g for 10 minutes at 4° C. and stored at −80° C. until use for analysis.

For immunoglobulin G (IgG) ELISA (Life Diagnostics Human IgG ELISA kit), the serum samples are diluted at 100 to 20,000 fold with 1× diluent solution. 100 μL of diluted serum is dispensed into anti-human IgG coated 96-well plate in duplicate alongside human IgG standards and incubated in a plate shaker at 150 rpm at 25° C. for 45 minutes. The wells are washed 5 times with 1× wash solution using a plate washer (400 μL/well). 100 μL of HRP conjugate is added into each well and incubated in a plate shaker at the same condition above. The wells are washed 5 times again with 1× wash solution using a plate washer (400 μL/well). 100 μL of TMB reagent is added into each well and incubated in a plate shaker at the same condition above. The reaction is stopped by adding 100 μL of Stop solution to each well. The absorbance is read at 450 nm (A450) with a microplate reader. The amount of human IgG in mouse serum is determined by plotting A450 values for the assay standard against human IgG concentration.

### Example 3

### Determination of pKa of Formulated Lipids

As described elsewhere, the pKa of formulated lipids is correlated with the effectiveness of LNPs for delivery of nucleic acids (see Jayaraman et al, Angewandte Chemie, International Edition (2012), 51(34), 8529-8533; Semple et al, Nature Biotechnology 28, 172-176 (2010)). In some embodiments, the preferred range of pKa is ˜5 to ˜7. The pKₐ of each lipid may be determined in lipid nanoparticles using an assay based on fluorescence of 2-(p-toluidino)-6-napthalene sulfonic acid (TNS). Lipid nanoparticles comprising compound of Formula (I)/DSPC/cholesterol/PEG-lipid (50:10:38.5:1.5 or 47.5:10:40.7:1.8 mol %) in PBS at a concentration of 0.4 mM total lipid are prepared using the in-line process as described in Example 1. TNS is prepared as a 100 μM stock solution in distilled water. Vesicles are diluted to 24 μM lipid in 2 mL of buffered solutions containing 10 mM HEPES, 10 mM MES, 10 mM ammonium acetate, and 130 mM NaCl, where the pH ranged from 2.5 to 11. An aliquot of the TNS solution is added to give a final concentration of 1 μM and following vortex mixing fluorescence intensity is measured at room temperature in a SLM Aminco Series 2 Luminescence Spectrophotometer using excitation and emission wavelengths of 321 nm and 445 nm. A sigmoidal best fit analysis is applied to the fluorescence data and the pKₐ is measured as the pH giving rise to half-maximal fluorescence intensity.

### Example 4

### Determination of Efficacy of Lipid Nanoparticle Formulations Containing Various Cationic Lipids Using IgG mRNA Expression Rodent Model

Representative compounds of the disclosure shown in Table 2 were formulated using the following molar ratio: 50% cationic lipid/10% distearoylphosphatidylcholine (DSPC)/38.5% Cholesterol/1.5% PEG lipid 2-[2-(ω-methoxy(polyethyleneglycol₂₀₀₀)ethoxy]-N,N-ditetradecylacetamide) or 47.5% cationic lipid/10% DSPC/40.7% Cholesterol/1.8% PEG lipid. Relative activity was determined by measuring the amount of human IgG in mouse serum as described in Example 2. The activity was compared at a dose of 1.0 or 0.3 mg mRNA/kg and expressed as ng luciferase/g liver measured 4 hours after administration, as described in Example 1 or as μg IgG/mL serum measured 24 hours after administration, as described in Example 2. Compound numbers in Table 2 refer to the compound numbers of Table 1.

TABLE 2
Novel Cationic Lipids and Associated Activity

| Compound | Serum IgG (ug/mL) | Serum IgG (ug/mL) |
|---|---|---|
| No. | @ 0.3 mg/kg | @ 1.0 mg/kg |
| I-1 | 18.5 ± 6.7 | — |
| I-2 | 23.2 ± 6.1 | 69.8 ± 18.2 |
| I-3 | 16.2 ± 4.3 | 52.7 ± 8.4 |
| I-4 | 24.2 ± 4.5 | — |
| I-5 | 25.9 ± 11.8 | 73.4 ± 14.9 |
| I-6 | 20.5 ± 4.0 | 197.5 ± 39.3 |
| I-7 | 30.8 ± 7.5 | — |
| I-8 | 10.6 ± 5.0 | — |
| I-9 | 13.4 ± 7.5 | 59.5 ± 25.6 |
| I-10 | 19.0 ± 3.3 | — |
| I-12 | 13.9 ± 4.7 | — |
| I-17 | 10.1 ± 4.03 | — |
| I-18 | 18.0 ± 2.0 | — |
| I-20 | 7.9 ± 3.6 | — |

### General Procedure A: Synthesis of Amide

To a mixture of the amine intermediate (1.0 eq), carboxylic acid (1.5 eq), DIPEA (3 eq) in DCM (0.2M) was added HATU (1.3 eq). The reaction was stirred at room temperature until reaction is complete (1-24 hours) The reaction mixture was concentrated then partitioned between EtOAc and sat. NaHCO₃. The organic layer was separated, dried over Na₂SO₄ and concentrated. Purification via flash chromatography (5% to 100% EtOAc in hexanes) gave the desired product.

### General Procedure B: Boc Deprotection

The boc-protected amine (1.0 eq) in TFA (0.08M) and DCM (0.08M) was stirred at room temperature for 2 h. The reaction was concentrated and the crude was partitioned between EtOAc and sat. NaHCO₃. The organic layer was separated, dried over Na₂SO₄ and concentrated to give the desired product which was used in the subsequent step without further purification.

### General Procedure C: Reductive Amination with Formaldehyde

A mixture of the amine intermediate (1.0 eq), 37 wt % formaldehyde in water (30 eq) in methanol (0.055M) was stirred at room temperature for 15 min. Sodium triacetoxyborohydride (4.2 eq) was added and the reaction was stirred at room temperature overnight. The reaction was quenched with sat. NaHCO₃ and the aqueous layer was extracted with EtOAc. Purification via flash chromatography (5% to 65% EtOAc in hexanes with 1% Et₃N) gave the desired compound.

### General Procedure D: Synthesis of Carbamoyl Chloride

To a mixture of the amine intermediate (1.0 eq) and sodium bicarbonate (4.0 eq) in DCM (0.1M) was added triphosgene (0.67 eq). The reaction mixture was stirred at room temperature overnight. The reaction solvent was decanted and then concentrated to give the desired product which was used in the next step without further purification.

### General Procedure E: Synthesis of Carbamate

The carbamoyl chloride intermediate (1.0 eq) was treated with the appropriate alcohol (10-20 eq) neat. The reaction was heated at 80° C. overnight. Purification via flash chromatography (5% to 100% EtOAc in hexanes with 1% Et₃N) followed by a second flash chromatography (2% to 10% MeOH in DCM) to give the desired compound.

### General Procedure F: Esterification

A mixture of the alcohol intermediate (1.0 eq), the appropriate carboxylic acid (1.05 eq), DCC (1.2 eq), 4-dimethylaminopyridine (1.0 eq), and triethylamine (4.0 eq) in DCM (0.11M) was stirred at room temperature overnight. Purification via flash chromatography (5% to 100% EtOAc in hexanes with 1% Et₃N) gave the desired compound. If the desired compound is not sufficiently pure, subsequent purifications via flash chromatography (2% to 10% MeOH in DCM or reverse phase c18 column with 50% to 100% MeOH in water plus 0.1% TFA) gave the desired compound.

### Synthetic Example 1

### Synthesis of N¹,N¹,N¹⁹,N¹⁹-tetrakis(decyl)-10-(N-decyl-3-(dimethylamino)propanamido)nonadecanediamide (Compound I-1)

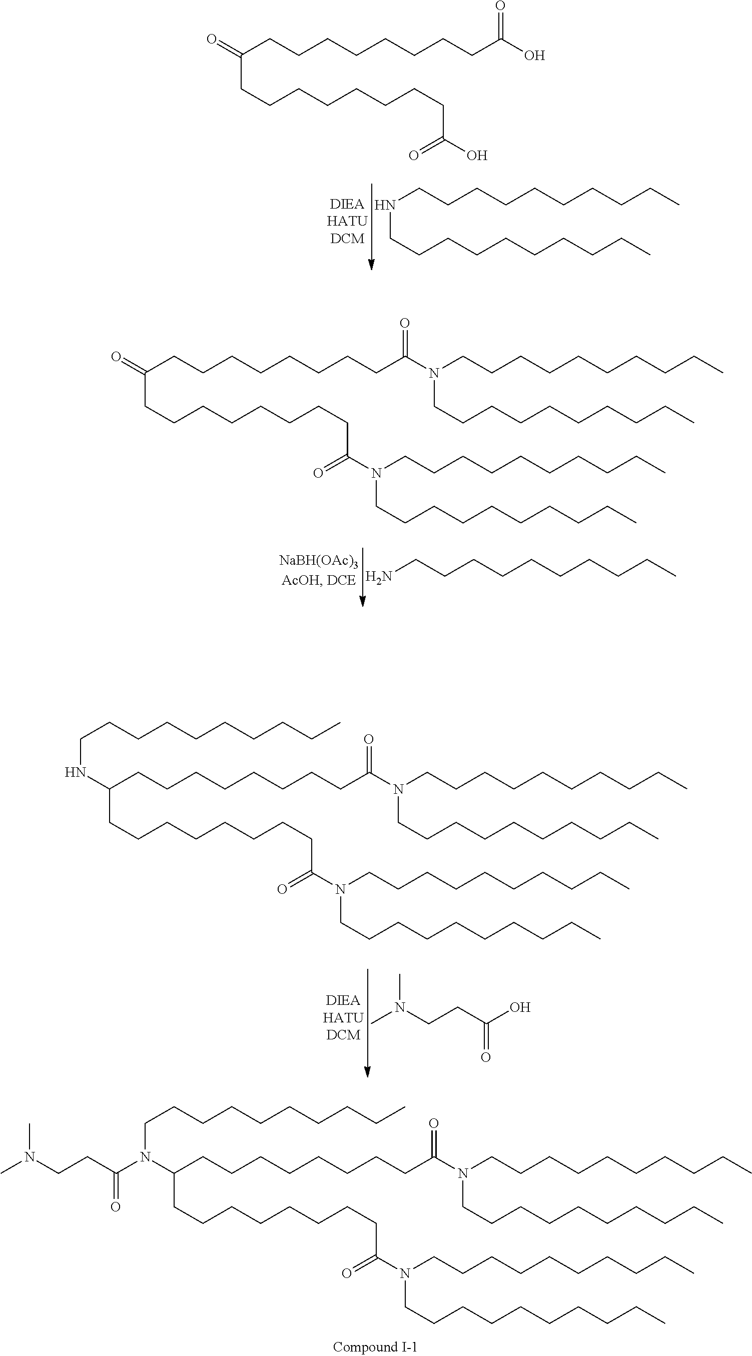

### Synthesis of N¹,N¹,N¹⁹,N¹⁹-tetrakis(decyl)-10-oxononadecanediamide

A mixture of 10-oxononadecanedioic acid (5.8 mmol, 2.0 g), dodecylamine (12.8 mmol, 3.8 g), DIEA (35 mmol, 6.1 mL) and HATU (15.2 mmol, 5.8 g) in DCM (29 mL) was stirred at room temperature overnight. The reaction mixture was concentrated and then partitioned between EtOAc and sat. NaHCO₃. The organic layer was separated, dried over Na₂SO₄ and concentrated. Purification via flash chromatography (5% to 50% EtOAc in hexanes) gave N¹,N¹,N¹⁹,N¹⁹-tetrakis(decyl)-10-oxononadecanediamide (4.3 g, 81%).

### Synthesis of N¹,N¹,N¹⁹,N¹⁹-tetrakis(decyl)-10-(decylamino)nonadecanediamide

A mixture of N¹,N¹,N¹⁹,N¹⁹-tetrakis(decyl)-10-oxononadecanediamide (1.7 mmol, 1.5 g), decylamine (2.5 mmol, 393 mg), acetic acid (2.5 mmol, 0.14 mL), and sodium triacetoxyborohydride (3.3 mmol, 705 mg) in dichloroethane (9.8 mL) was stirred at room temperature overnight. The reaction was quenched with saturated NaHCO₃ and extracted with ethyl acetate (EtOAc). The organic layer was separated, dried over Na₂SO₄ and concentrated. Purification via flash chromatography (10% to 100% EtOAc in hexanes) gave N¹,N¹,N¹⁹,N¹⁹-tetrakis(decyl)-10-(decylamino)nonadecanediamide (1.17 g, 67%).

### Synthesis of N¹,N¹,N¹⁹,N¹⁹-tetrakis(decyl)-10-(N-decyl-3-(dimethylamino)propanamido) nonadecanediamide (Compound I-1)

To a mixture of the Synthesis of N¹,N¹,N¹⁹,N¹⁹-tetrakis(decyl)-10-(decylamino)nonadecanediamide (1.0 eq), 3-(dimethylamino)propanoic acid (1.5 eq), N,N-Diisopropylethylamine (DIEA or DIPEA; 3 eq) in DCM (0.2M) was added HATU (1.3 eq). The reaction was stirred at room temperature until reaction is complete. The reaction mixture was concentrated then partitioned between EtOAc and saturated NaHCO₃. The organic layer was separated, dried over Na₂SO₄ and concentrated. Purification was performed via flash chromatography (5% to 65% EtOAc in hexanes with 1% Et₃N) to give the desired product. Yield (51 mg, 46%).

¹H NMR (400 MHz, CDCl₃) δ 3.60 (t, J=7.1 Hz, 1H), 3.32-3.22 (m, 4H), 3.24-3.14 (m, 4H), 3.09-2.99 (m, 2H), 2.72-2.58 (m, 2H), 2.53-2.43 (m, 2H), 2.31-2.20 (m, 10H), 1.70-1.37 (m, 22H), 1.36-1.07 (m, 93H), 0.96-0.80 (m, 15H). m/z calcd. for Chemical Formula: C₇₄H₁₄₈N₄O₃=1141.2. Found [M+H]⁺=1142.5.

### Synthetic Example 2

### Synthesis of N,N-didecyl-8-(N-(8-(didecylamino)-8-oxooctyl)-4-(dimethylamino)-2-fluorobutanamido)octanamide (Compound I-2)

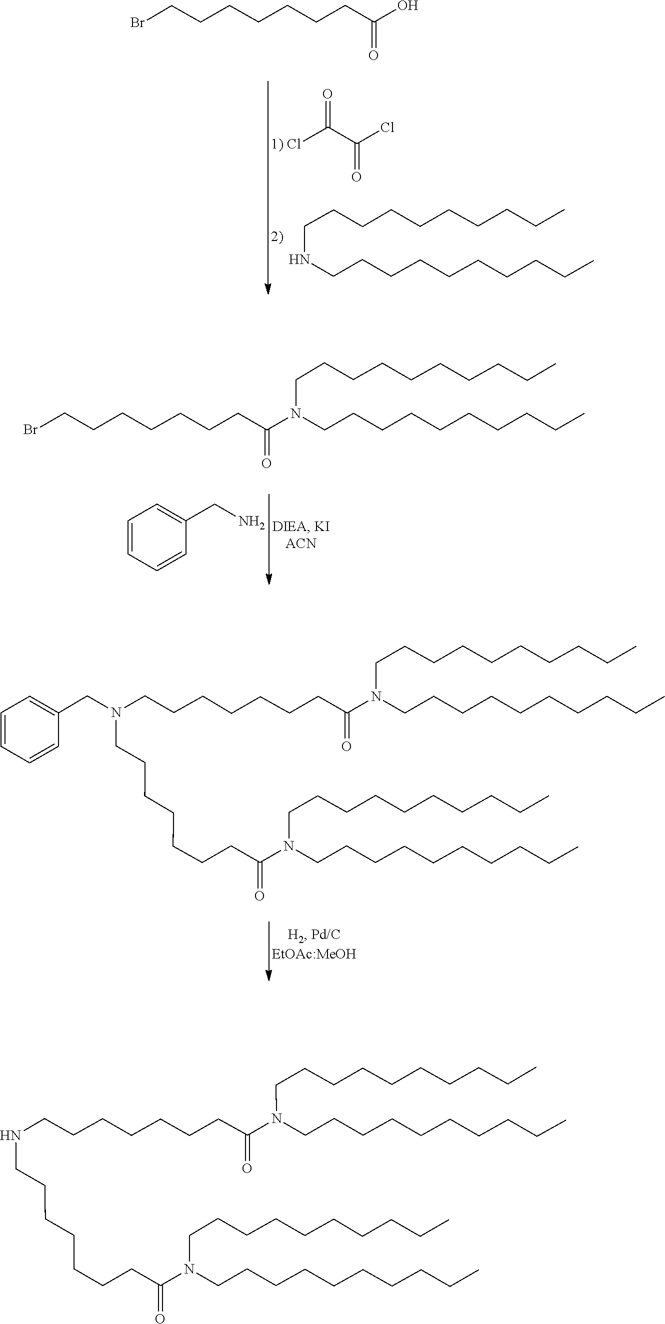

### Synthesis of 8-bromo-N,N-didecyloctanamide

To a mixture of 8-bromooctanoic acid (47.3 mmol, 10.5 g) and DMF (cat.) in DCM (100 mL) was added oxalyl chloride (142 mmol, 18.0 g) and the reaction mixture was stirred at room temperature for 1 h. The reaction mixture was concentrated to give 8-bromooctanoyl chloride which was used in the next step without further purification.

To a mixture of didecylamine (69.1 mmol, 20.6 g), triethylamine (377 mmol, 52.5 mL), and N,N-dimethylpyridin-4-amine (cat. 8.0 mg) in DCM (100 mL) was added 8-bromooctanoyl chloride (62.8 mmol, 15.2 g) in DCM (60 mL) and the reaction mixture was stirred at room temperature for 17 h. The reaction mixture was concentrated, and the crude material was resuspended in hexanes. The resultant solids were filtered and the filtrate was purified via automated flash chromatography (5% to 25% EtOAc in hexanes) to give 8-bromo-N,N-didecyloctanamide (26 g, 82%).

### Synthesis of 8,8′-(benzylazanediyl)bis(N,N-didecyloctanamide)

A mixture of 8-bromo-N,N-didecyloctanamide (2.8 mmol, 1.4 g), benzylamine (1.7 mmol, 0.18 mL), DIEA (5.0 mmol, 0.88 mL), and potassium iodide (3.9 mmol, 647 mg) in acetonitrile (18 mL) was heated at 75° C. for 19 h. The reaction mixture was concentrated and the crude was suspended in hexanes:EtOAc:Et₃N (95:5:1) and filtered. The filtrate was purified via flash chromatography (5% to 65% EtOAc in hexanes with 1% Et₃N) to give 8,8′-(benzylazanediyl)bis(N,N-didecyloctanamide) (605 mg, 45%).

### Synthesis of 8,8′-azanediylbis(N,N-didecyloctanamide)

A mixture of 8,8′-(benzylazanediyl)bis(N,N-didecyloctanamide) (0.63 mmol, 600 mg) and 10 wt % Pd/C (0.06 mmol, 66 mg) in EtOAc:MeOH (5 mL:15 mL) was stirred under H₂ gas (balloon pressure) at room temperature overnight. The reaction mixture was filtered over a pad of diatomaceous earth (e.g., Celite®) and the filter bed was washed with EtOAc. The filtrate was concentrated to give 8,8′-azanediylbis(N,N-didecyloctanamide) (550 mg, quantitative) which was used in the subsequent step without further purification.

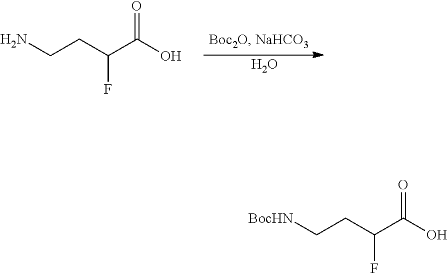 [SMILES: NCCC(F)C(=O)O ; O ; CC(C)(C)OC(=O)NCCC(F)C(=O)O]

### Synthesis of 4-((tert-butoxycarbonyl)amino)-2-fluorobutanoic acid

A mixture of 4-amino-2-fluorobutanoic acid hydrochloride (3.8 mmol, 600 mg), di-tert-butyl decarbonate (4.6 mmol, 1.0 g), and sodium bicarbonate (7.6 mmol, 640 mg) in water (10 mL) was stirred at room temperature for 4 h. The reaction mixture was washed with EtOAc and the aqueous layer was acidified with 0.1 M HCl. The aqueous layer was extracted with EtOAc to give 4-((tert-butoxycarbonyl)amino)-2-fluorobutanoic acid (75 mg, 9%).

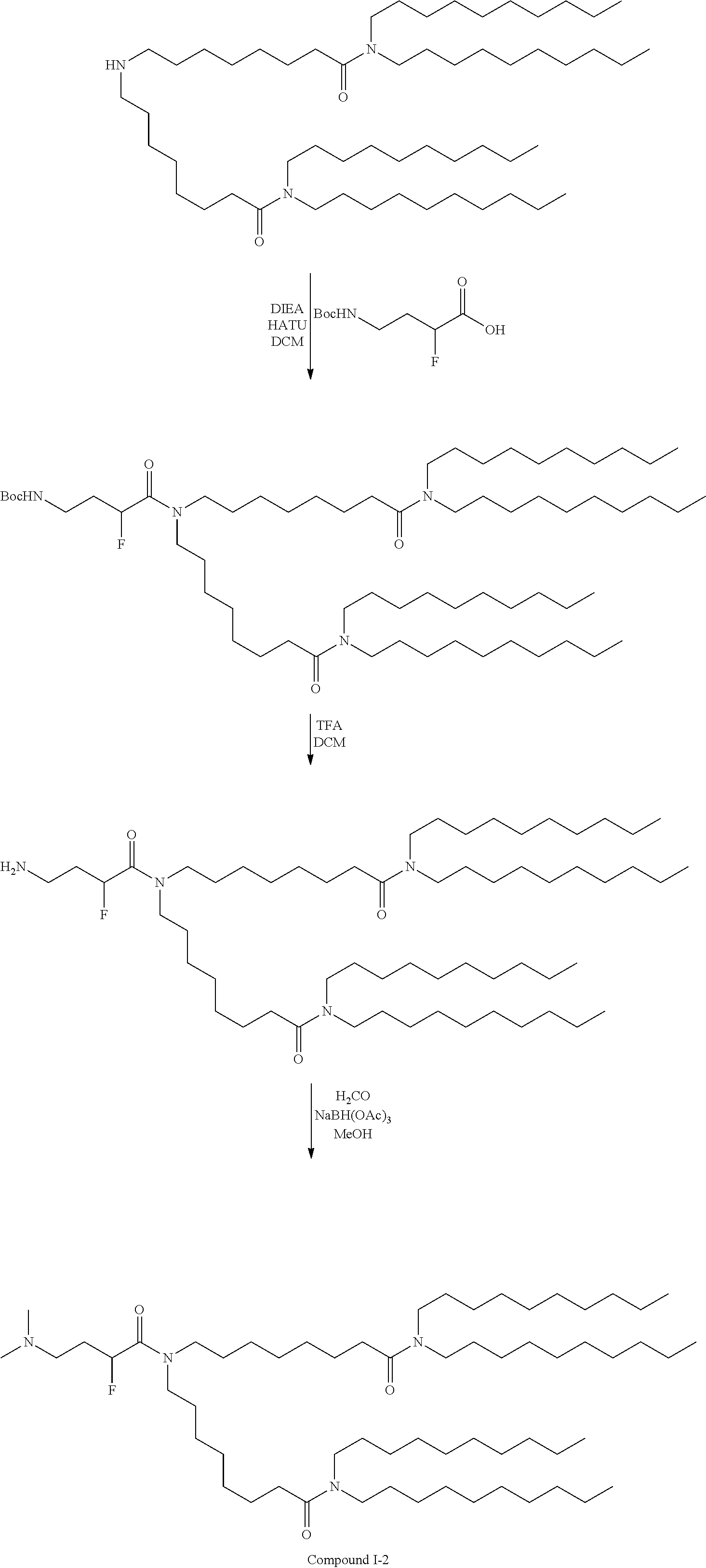

### Synthesis of tert-butyl (4-(bis(8-(didecylamino)-8-oxooctyl)amino)-3-fluoro-4-oxobutyl)carbamate

To a mixture of the 8,8′-azanediylbis(N,N-didecyloctanamide) (1.0 eq), 4-((tert-butoxycarbonyl)amino)-2-fluorobutanoic acid (1.5 eq), DIEA (3 eq) in DCM (0.2M) was added HATU (1.3 eq). The reaction was stirred at room temperature until reaction is complete. The reaction mixture was concentrated then partitioned between EtOAc and saturated NaHCO₃. The organic layer was separated, dried over Na₂SO₄ and concentrated. Purification via flash chromatography (5% to 100% EtOAc in hexanes) gave tert-butyl (4-(bis(8-(didecylamino)-8-oxooctyl)amino)-3-fluoro-4-oxobutyl)carbamate (90 mg, 65%).

### Synthesis of 8-(4-amino-N-(8-(didecylamino)-8-oxooctyl)-2-fluorobutanamido)-N,N-didecyloctanamide

Tert-butyl (4-(bis(8-(didecylamino)-8-oxooctyl)amino)-3-fluoro-4-oxobutyl)carbamate (1.0 eq) in TFA (0.08M) and DCM (0.08M) was stirred at room temperature for 2 h. The reaction was concentrated, and the crude was partitioned between EtOAc and sat. NaHCO₃. The organic layer was separated, dried over Na₂SO₄ and concentrated to give 8-(4-amino-N-(8-(didecylamino)-8-oxooctyl)-2-fluorobutanamido)-N,N-didecyloctanamide (72 mg, 88%) which was used in the subsequent step without further purification.

### Synthesis of N,N-didecyl-8-(N-(8-(didecylamino)-8-oxooctyl)-4-(dimethylamino)-2-fluorobutanamido)octanamide (Compound I-2)

A mixture of the 8-(4-amino-N-(8-(didecylamino)-8-oxooctyl)-2-fluorobutanamido)-N,N-didecyloctanamide (1.0 eq), 37 wt % formaldehyde in water (30 eq) in methanol (0.055M) was stirred at room temperature for 15 min. Sodium triacetoxyborohydride (4.2 eq) was added and the reaction was stirred at room temperature overnight. The reaction was quenched with sat. NaHCO₃ and the aqueous layer was extracted with EtOAc. Purification via flash chromatography (5% to 65% EtOAc in hexanes with 1% Et₃N) gave N,N-didecyl-8-(N-(8-(didecylamino)-8-oxooctyl)-4-(dimethylamino)-2-fluorobutanamido)octanamide (27 mg, 36%).

¹H NMR (600 MHz, CDCl₃) δ 5.22 (ddd, J=49.2, 8.1, 4.7 Hz, 1H), 3.41-3.35 (m, 1H), 3.33-3.24 (m, 6H), 3.24-3.16 (m, 4H), 2.45-2.35 (m, 2H), 2.35-2.23 (m, 9H), 2.11-1.95 (m, 2H), 1.69-1.48 (m, 18H), 1.41-1.15 (m, 69H), 0.96-0.86 (m, 12H). m/z calcd. for Chemical Formula: C₆₂H₁₂₃FN₄O₃=991.0. Found [M+H]⁺=992.0.

### Synthetic Example 3

### N-decyl-N-(8-(N-(8-(didecylamino)-8-oxooctyl)-4-(dimethylamino)-2-fluorobutanamido)octyl)decanamide (Compound I-3)

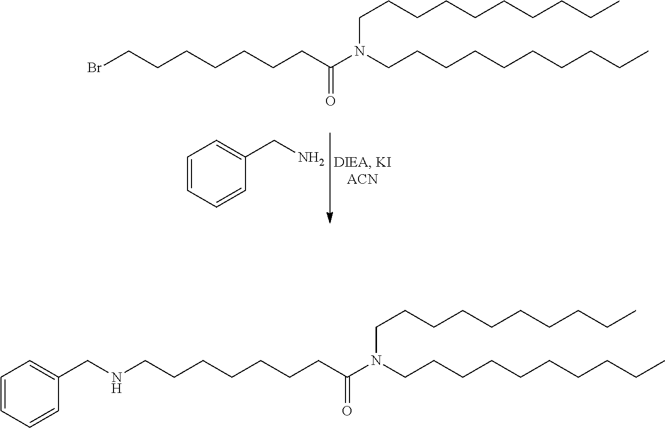 [SMILES: CCCCCCCCCCN(CCCCCCCCCC)C(=O)CCCCCCCBr ; NCc1ccccc1 ; CCCCCCCCCCN(CCCCCCCCCC)C(=O)CCCCCCCNCc1ccccc1]

### Synthesis of 8-(benzylamino)-N,N-didecyloctanamide

A mixture of 8-bromo-N,N-didecyloctanamide (4.0 mmol, 2.0 g), benzylamine (10 mmol, 1.1 mL), DIEA (6.0 mmol, 1.05 mL), and potassium iodide (6.0 mmol, 991 mg) in acetonitrile (4.0 mL) was heated via microwave irradiation at 140° C. for 30 min. The reaction mixture was concentrated and the crude was suspended in hexanes:EtOAc:Et₃N (95:5:1) and filtered. The filtrate was purified via flash chromatography (5% to 100% EtOAc in hexanes with 1% Et₃N) to give 8-(benzylamino)-N,N-didecyloctanamide (1.56 g, 74%).

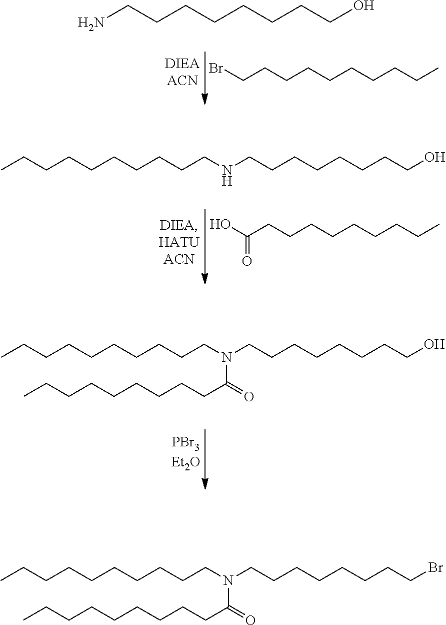 [SMILES: NCCCCCCCCO ; CCCCCCCCCCBr ; CCCCCCCCCCNCCCCCCCCO ; CCCCCCCCCC(=O)O ; CCCCCCCCCCN(CCCCCCCCO)C(=O)CCCCCCCCC ; CCCCCCCCCCN(CCCCCCCCBr)C(=O)CCCCCCCCC]

### Synthesis of 8-(decylamino)octan-1-ol

A mixture of 8-aminooctan-1-ol (136 mmol, 19.7 g), 1-bromodecane (45.2 mmol, 10 g), and DIEA (140 mmol, 24.4 mL) in acetonitrile (90 mL) was heated at 75° C. overnight. The reaction mixture was concentrated then partitioned between EtOAc and sat. NaHCO₃. The organic layer was separated, dried over Na₂SO₄ and concentrated. Purification via flash chromatography (5% to 100% EtOAc in hexanes with 1% Et₃N) gave 8-(decylamino)octan-1-ol (7.2 g, 56%).

### Synthesis of N-decyl-N-(8-hydroxyoctyl)decanamide

A mixture of 8-(decylamino)octan-1-ol (14 mmol, 4 g), decanoic acid (14 mmol, 2.4 g), DIEA (42 mmol, 7.3 mL) and HATU (18.2 mmol, 6.9 g) in DCM (28 mL) was stirred at room temperature overnight. The reaction mixture was concentrated then partitioned between EtOAc and sat. NaHCO₃. The organic layer was separated, dried over Na₂SO₄ and concentrated. Purification via flash chromatography (5% to 100% EtOAc in hexanes) gave N-decyl-N-(8-hydroxyoctyl)decanamide (4.0 g, 65%).

### Synthesis of N-(8-bromooctyl)-N-decyldecanamide

To a solution of N-decyl-N-(8-hydroxyoctyl)decanamide (9.6 mmol, 4.2 g) in diethyl ether (19 mL) was added phosphorous tribromide (28.7 mmol, 2.7 mL) dropwise at 5° C. The reaction mixture was allowed to warm to room temperature and stirred at room temperature for 72 h. The reaction was quenched with water and extracted with EtOAc. The organic layer was separated, dried over Na₂SO₄ and concentrated. Purification via flash chromatography (0% to 20% EtOAc in hexanes) gave N-(8-bromooctyl)-N-decyldecanamide (3.0 g, 63%).

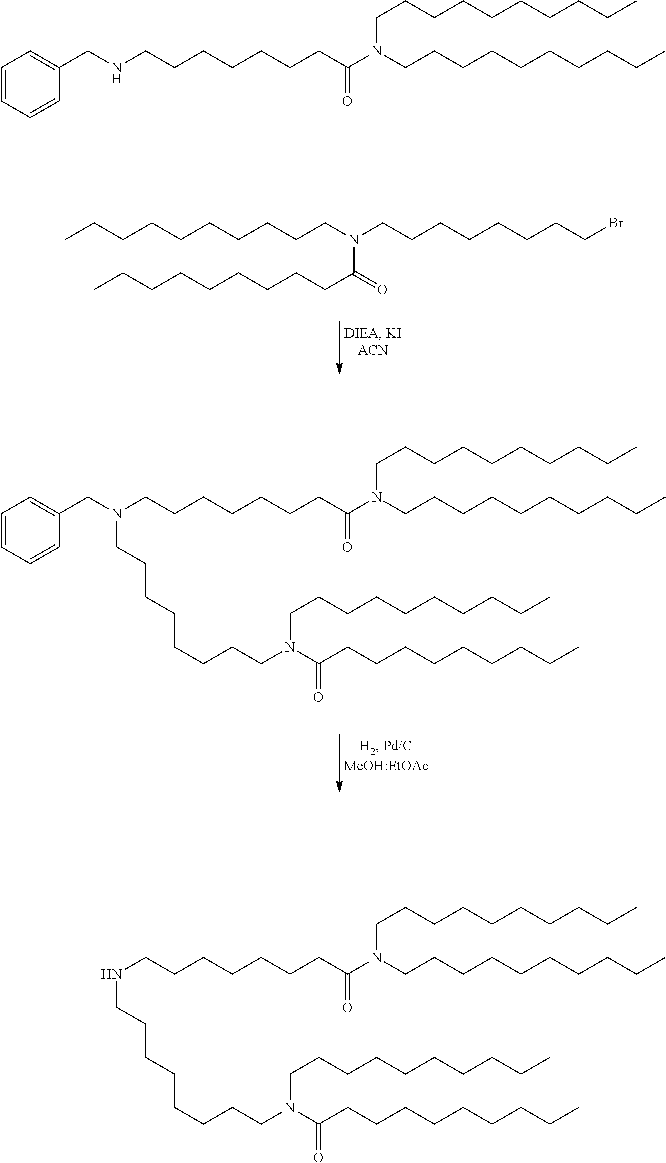

### Synthesis of N-(8-(benzyl(8-(didecylamino)-8-oxooctyl)amino)octyl)-N-decyldecanamide

A mixture of 8-(benzylamino)-N,N-didecyloctanamide (0.95 mmol, 0.5 g), N-(8-bromooctyl)-N-decyldecanamide (0.95 mmol, 475 mg), DIEA (2.9 mmol, 0.51 mL), and potassium iodide (2.8 mmol, 471 mg) in acetonitrile was heated via microwave irradiation at 140° C. for 30 min. The reaction mixture was concentrated and the crude was suspended in hexanes:EtOAc:Et₃N (95:5:1) and filtered. The filtrate was purified via flash chromatography (5% to 65% EtOAc in hexanes with 1% Et₃N) to give N-(8-(benzyl(8-(didecylamino)-8-oxooctyl)amino)octyl)-N-decyldecanamide (698 mg, 78%).

### Synthesis of N-decyl-N-(8-((8-(didecylamino)-8-oxooctyl)amino)octyl)decanamide

N-decyl-N-(8-((8-(didecylamino)-8-oxooctyl)amino)octyl)decanamide was prepared from N-(8-(benzyl(8-(didecylamino)-8-oxooctyl)amino)octyl)-N-decyldecanamide according to the to the general procedures of Synthetic Example 2. Yield (630 mg, quantitative).

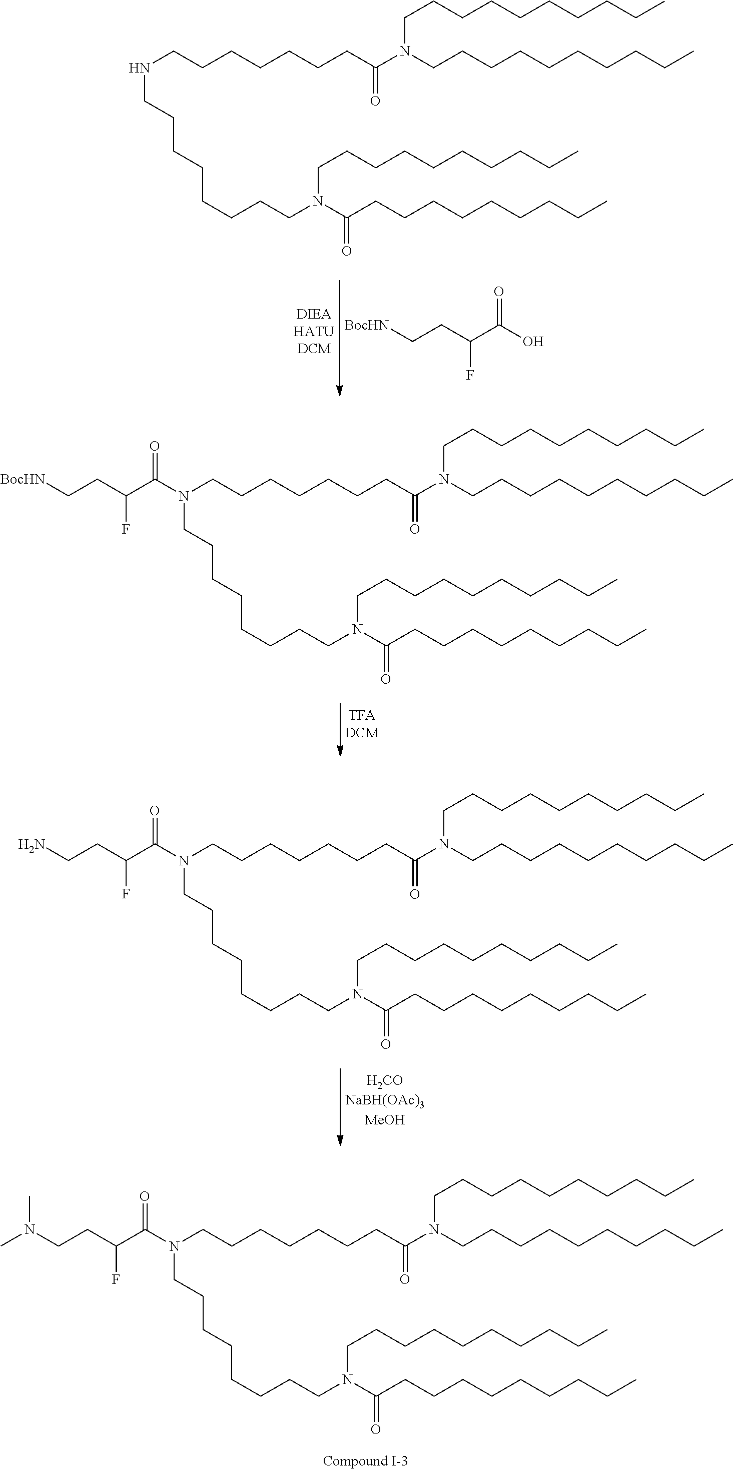

### Synthesis of tert-butyl (4-((8-(N-decyldecanamido)octyl)(8-(didecylamino)-8-oxooctyl)amino)-3-fluoro-4-oxobutyl)carbamate

Tert-butyl (4-((8-(N-decyldecanamido)octyl)(8-(didecylamino)-8-oxooctyl)amino)-3-fluoro-4-oxobutyl)carbamate was prepared from N-decyl-N-(8-((8-(didecylamino)-8-oxooctyl)amino)octyl)decanamide and 4-((tert-butoxycarbonyl)amino)-2-fluorobutanoic acid according to the general procedures of Synthetic Example 1. Yield (113 mg, 76%).

### Synthesis of N-(8-(4-amino-N-(8-(didecylamino)-8-oxooctyl)-2-fluorobutanamido)octyl)-N-decyldecanamide

N-(8-(4-amino-N-(8-(didecylamino)-8-oxooctyl)-2-fluorobutanamido)octyl)-N-decyldecanamide was prepared from tert-butyl (4-((8-(N-decyldecanamido)octyl)(8-(didecylamino)-8-oxooctyl)amino)-3-fluoro-4-oxobutyl)carbamate according to the general procedures of Synthetic Example 2.

### Synthesis of N-decyl-N-(8-(N-(8-(didecylamino)-8-oxooctyl)-4-(dimethylamino)-2-fluorobutanamido)octyl)decanamide (Compound I-3)

A mixture of N-(8-(4-amino-N-(8-(didecylamino)-8-oxooctyl)-2-fluorobutanamido)octyl)-N-decyldecanamide (1.0 eq), 37 wt % formaldehyde in water (30 eq) in methanol (0.055M) was stirred at room temperature for 15 min. Sodium triacetoxyborohydride (4.2 eq) was added and the reaction was stirred at room temperature overnight. The reaction was quenched with sat. NaHCO₃ and the aqueous layer was extracted with EtOAc. Purification via flash chromatography (5% to 65% EtOAc in hexanes with 1% Et₃N) gave Compound I-3 (58 mg, 53%).

¹H NMR (400 MHz, CDCl₃) δ 5.31-5.08 (m, 1H), 3.44-3.10 (m, 12H), 2.55-2.33 (m, 2H), 2.31-2.19 (m, 10H), 2.12-1.90 (m, 2H), 1.78-1.42 (m, 21H), 1.38-1.19 (m, 69H), 0.99-0.78 (m, 12H). m/z calcd. for Chemical Formula: C₆₂H₁₂₃FN₄O₃=990.1. Found [M+H]⁺=992.3.

### Synthetic Example 4

### N,N-didecyl-8-(N-(8-(didecylamino)-8-oxooctyl)-2-(dimethylamino)acetamido)octanamide (Compound I-4)

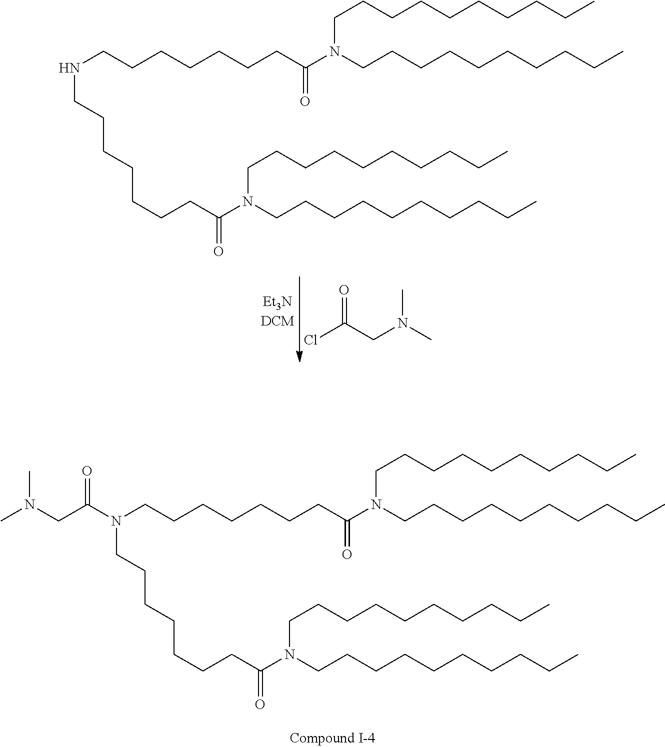

### Synthesis of N,N-didecyl-8-(N-(8-(didecylamino)-8-oxooctyl)-2-(dimethylamino)acetamido) octanamide (Compound I-4)

A mixture of intermediate 8,8′-azanediylbis(N,N-didecyloctanamide) (0.12 mmol, 100 mg), dimethylglycinoyl chloride hydrochloride (0.38 mmol, 60 mg), triethylamine (0.97 mmol, 0.14 mL), and 4-dimethylaminopyridine (cat.) in DCM (5 mL) was stirred at room temperature for 24 h. The reaction mixture was purified via flash chromatography (5% to 65% EtOAc in hexanes with 1% Et₃N) to give Compound I-4 (79 mg, 66%).

¹H NMR (400 MHz, CDCl₃) δ 3.36-3.23 (m, 8H), 3.23-3.13 (m, 4H), 3.06 (s, 2H), 2.32-2.20 (m, 10H), 1.69-1.58 (m, 7H), 1.57-1.43 (m, 12H), 1.38-1.19 (m, 69H), 0.92-0.82 (m, 12H). m/z calcd. for Chemical Formula: C₆₀H₁₂₀N₄O₃=944.9. Found [M+H]⁺=946.1.

### Synthetic Example 5

### N-decyl-N-(8-(N-(8-(didecylamino)-8-oxooctyl)-2-(dimethylamino)acetamido)octyl)decanamide (Compound I-5)

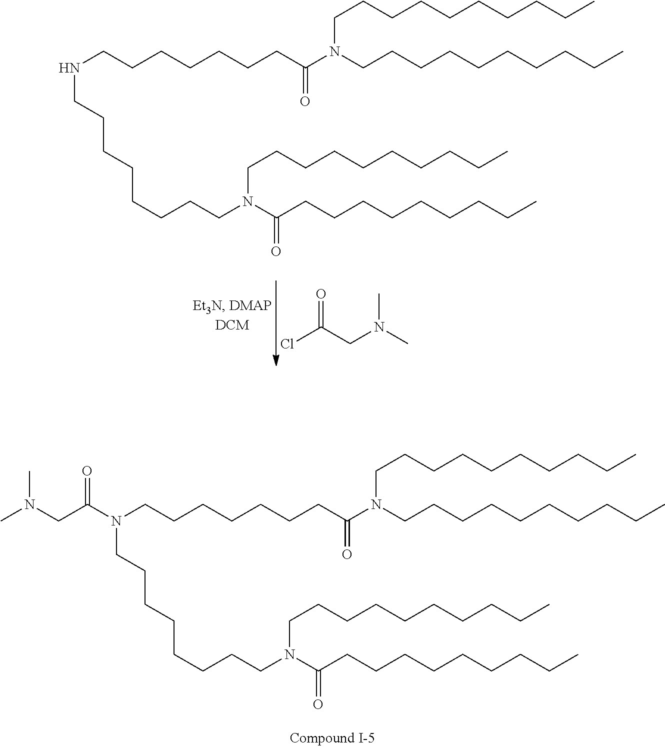

### Synthesis of N-decyl-N-(8-(N-(8-(didecylamino)-8-oxooctyl)-2-(dimethylamino)acetamido)octyl)decanamide (Compound I-5)

A mixture of N-decyl-N-(8-((8-(didecylamino)-8-oxooctyl)amino)octyl)decanamide (0.12 mmol, 100 mg), dimethylglycinoyl chloride hydrochloride (0.13 mmol, 20 mg), and triethylamine (0.26 mmol, 0.036 mL) in DCM (1 mL) was stirred at room temperature overnight. An additional portion of dimethylglycinoyl chloride hydrochloride (0.13 mmol, 20 mg), triethylamine (0.5 mL), and DCM (2 mL) was added, and the reaction mixture was stirred at room temperature for 3 h. Purification via flash chromatography (5% to 65% EtOAc in hexanes with 1% Et₃N) gave Compound I-5 (61 mg, 55%).

¹H NMR (400 MHz, CDCl₃) δ 3.35-3.24 (m, 8H), 3.23-3.14 (m, 4H), 3.07 (s, 2H), 2.32-2.20 (m, 10H), 1.68-1.59 (m, 8H), 1.58-1.42 (m, 13H), 1.40-1.16 (m, 68H), 0.94-0.78 (m, 12H). m/z calcd. for Chemical Formula: C₆₀H₁₂₀N₄O₃=944.9. Found [M+H]⁺=946.2.

### Synthetic Example 6

### 2-(dimethylamino)ethyl bis(8-(didecylamino)-8-oxooctyl)carbamate (Compound I-6)

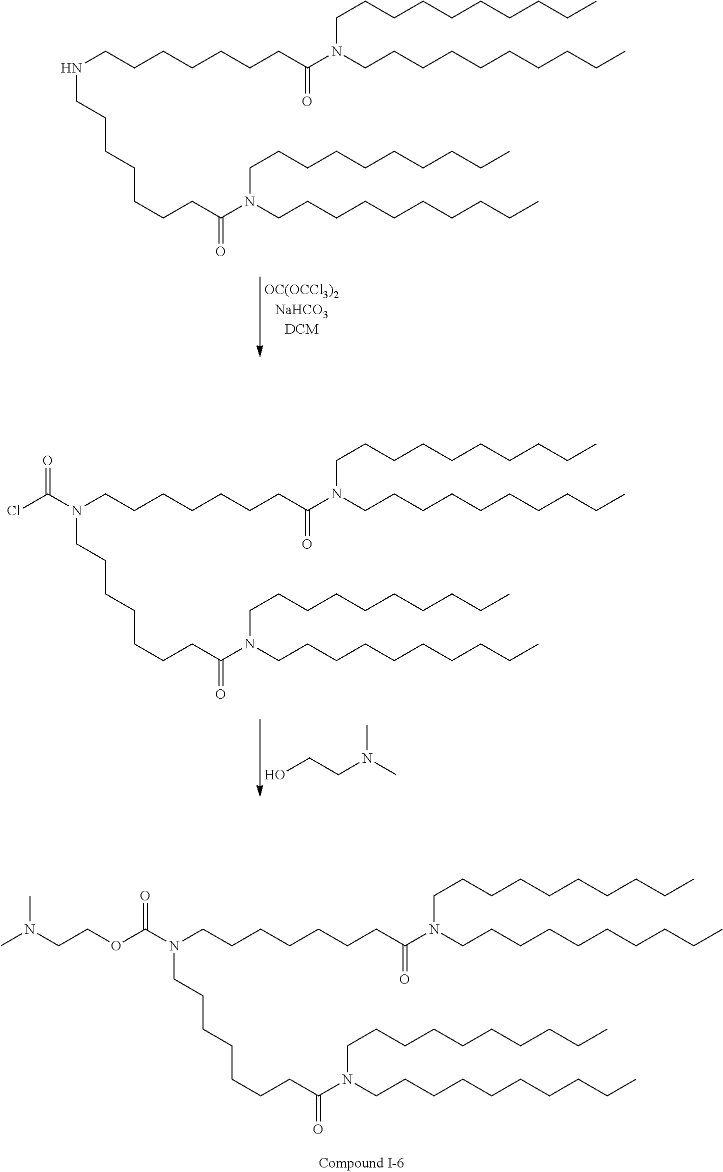

### Synthesis of bis(8-(didecylamino)-8-oxooctyl)carbamic chloride

To a mixture of 8,8′-azanediylbis(N,N-didecyloctanamide) (1.0 eq) and sodium bicarbonate (4.0 eq) in DCM (0.1M) was added triphosgene (0.67 eq). The reaction mixture was stirred at room temperature overnight. The reaction solvent was decanted and then concentrated to give the desired product which was used in the next step without further purification.

### Synthesis of 2-(dimethylamino)ethyl bis(8-(didecylamino)-8-oxooctyl)carbamate (Compound I-6)

Bis(8-(didecylamino)-8-oxooctyl)carbamic chloride (1.0 eq) was treated with the 2-(dimethylamino)ethan-1l-ol (10-20 eq) neat. The reaction was heated at 80° C. overnight. Purification via flash chromatography (5% to 100% EtOAc in hexanes with 1% Et₃N) followed by a second flash chromatography (2% to 10% MeOH in DCM) to give Compound I-6. Yield (21 mg, 15%).

¹H NMR (400 MHz, CDCl₃) δ 4.17 (t, J=6.0 Hz, 2H), 3.34-3.24 (m, 4H), 3.23-3.08 (m, 7H), 2.62-2.54 (m, 2H), 2.35-2.20 (m, 9H), 1.72-1.58 (m, 7H), 1.58-1.43 (m, 12H), 1.38-1.18 (m, 70H), 0.93-0.82 (m, 12H). m/z calcd. for Chemical Formula: C₆₁H₁₂₂N₄O₄=975.0. Found [M+H]⁺=975.9.

### Synthetic Example 7

### 3-(dimethylamino)propyl bis(8-didecylamino)-8-oxooctyl)carbamate (Compound I-7)

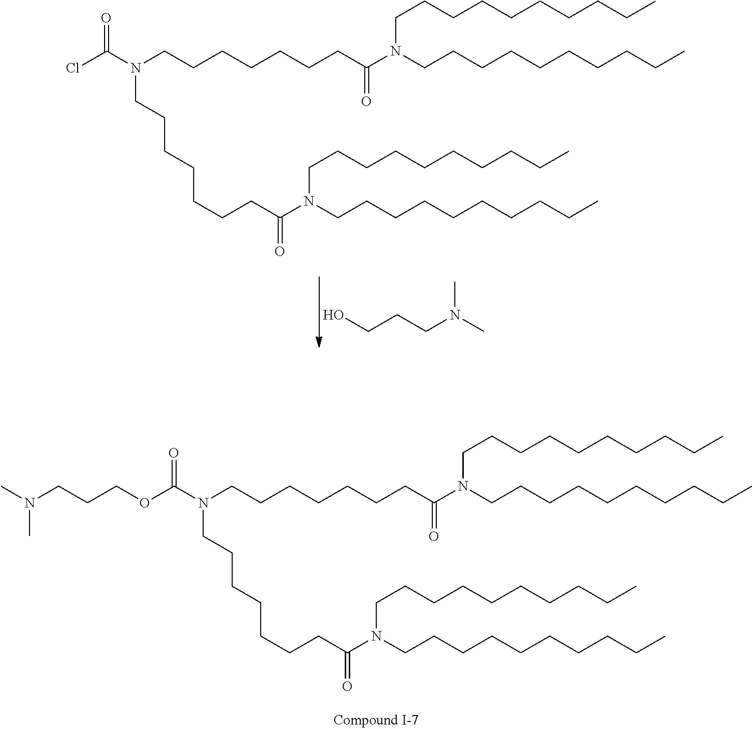

### Synthesis of 3-(dimethylamino)propyl bis(8-(didecylamino)-8-oxooctyl)carbamate (Compound I-7)

Compound I-7 was prepared from intermediate bis(8-(didecylamino)-8-oxooctyl)carbamic chloride and 3-(dimethylamino)propan-1-ol according to the general procedures of Synthetic Example 6. Yield (60 mg, 22%).

¹H NMR (400 MHz, CDCl₃) δ 4.09 (t, J=6.4 Hz, 2H), 3.32-3.23 (m, 4H), 3.24-3.09 (m, 8H), 2.39-2.30 (m, 2H), 2.31-2.18 (m, 10H), 1.79 (dq, J=8.4, 6.5 Hz, 2H), 1.67-1.56 (m, 6H), 1.58-1.41 (m, 13H), 1.39-1.15 (m, 71H), 0.93-0.82 (m, 12H). m/z calcd. for Chemical Formula: C₆₂H₁₂₄N₄O₄=989.0. Found [M+H]⁺=990.0.

### Synthetic Example 8

### 3-(dimethylamino)propyl bis(10-(didecylamino)-10-oxodecyl)carbamate (Compound I-8)

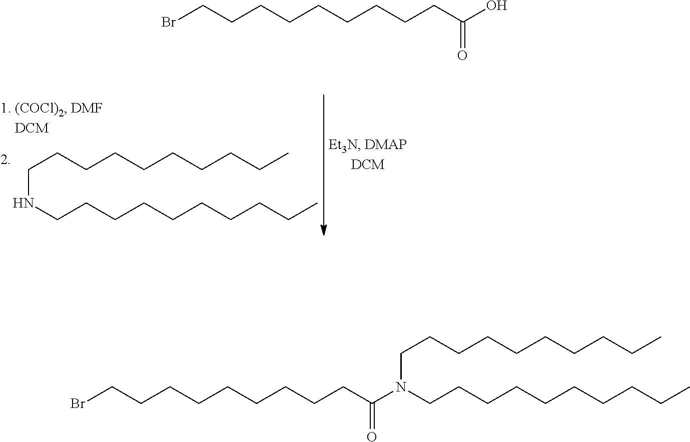 [SMILES: O=C(O)CCCCCCCCCBr ; CCCCCCCCCCNCCCCCCCCCC ; CCCCCCCCCCN(CCCCCCCCCC)C(=O)CCCCCCCCCBr]

### Synthesis of 10-bromo-N,N-didecyldecanamide

10-Bromo-N,N-didecyldecanamide was prepared from decanoic acid and didecylamine according to the general procedures Synthetic Example 2. Yield (5.1 g, 40%, 2 steps).

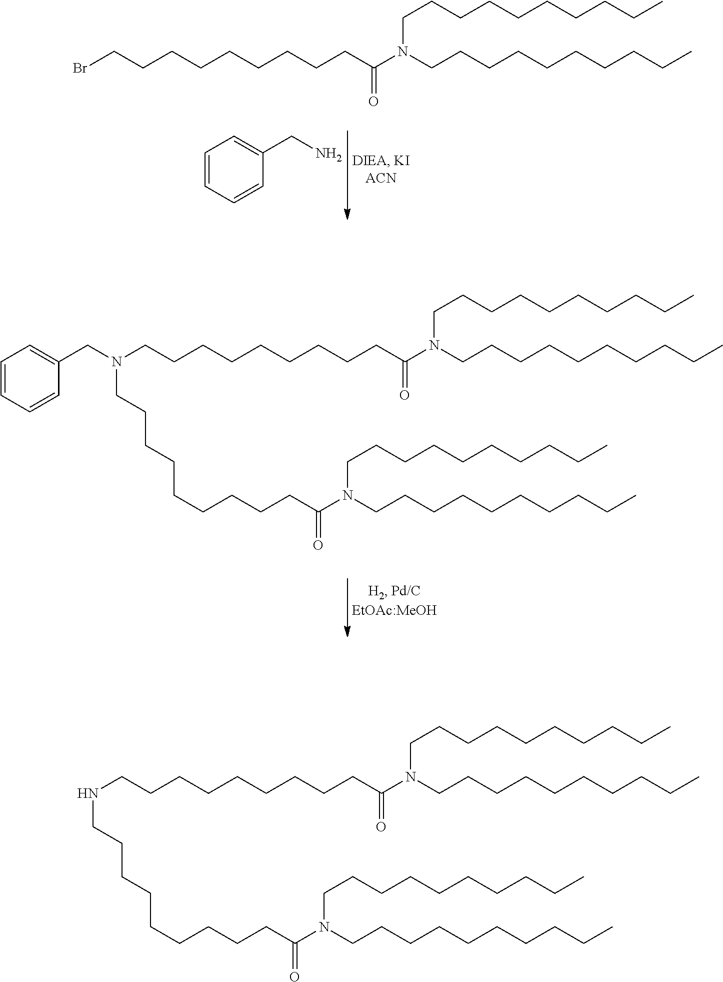 [SMILES: CCCCCCCCCCN(CCCCCCCCCC)C(=O)CCCCCCCCCBr ; NCc1ccccc1 ; CCCCCCCCCCN(CCCCCCCCCC)C(=O)CCCCCCCCCN(CCCCCCCCCC(=O)N(CCCCCCCCCC)CCCCCCCCCC)Cc1ccccc1 ; CCCCCCCCCCN(CCCCCCCCCC)C(=O)CCCCCCCCCNCCCCCCCCCC(=O)N(CCCCCCCCCC)CCCCCCCCCC]

### Synthesis of 10,10′-azanediylbis(N,N-didecyldecanamide)

10,10′-Azanediylbis(N,N-didecyldecanamide) was prepared according to the general procedures Synthetic Example 2. Yield (1.3 g, 98%).

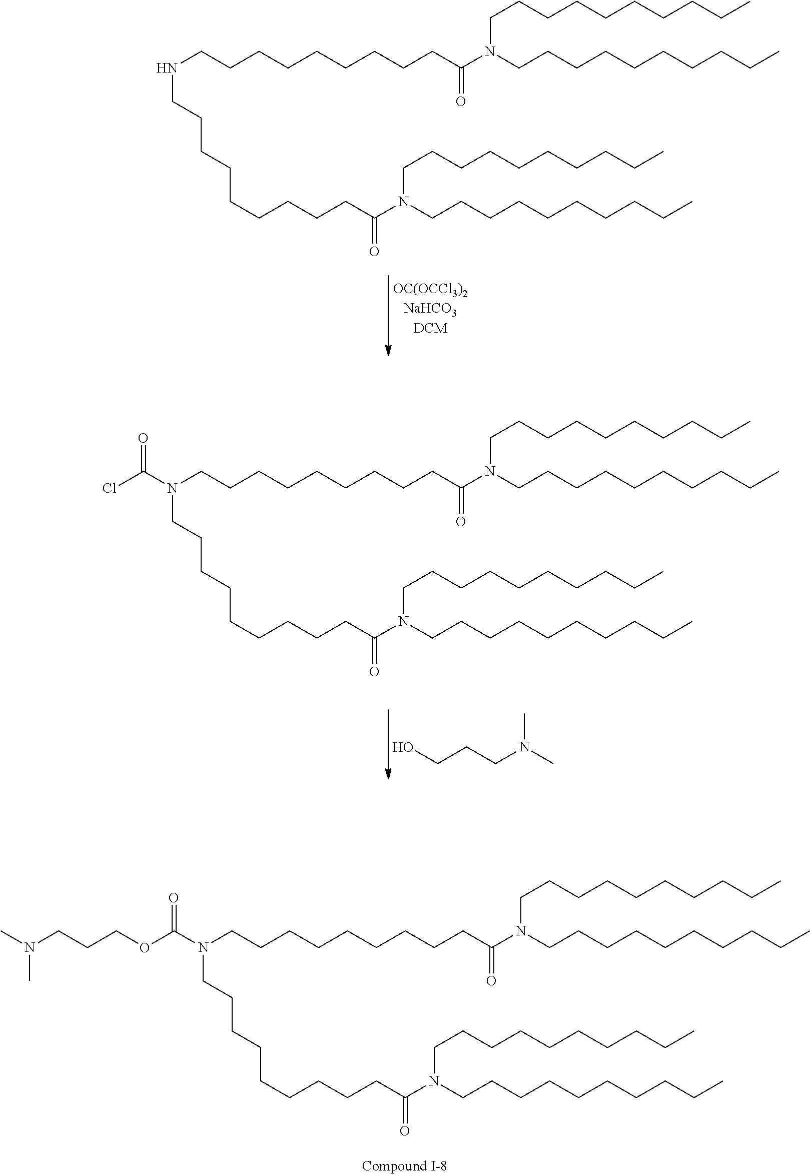

### Synthesis of bis(10-(didecylamino)-10-oxodecyl)carbamic chloride

Bis(10-(didecylamino)-10-oxodecyl)carbamic chloride was prepared according to the general procedures Synthetic Example 6. Yield (325 mg, quantitative).

### Synthesis of 3-(dimethylamino)propyl bis(10-(didecylamino)-10-oxodecyl)carbamate (Compound I-8)

A mixture of bis(10-(didecylamino)-10-oxodecyl)carbamic chloride (0.33 mmol, 325 mg) and 3-(dimethylamino)propan-1-ol (2.65 mmol, 274 mg) was heated at 75° C. for 19 h. Purification via flash chromatography (5% to 100% EtOAc in hexanes with 1% Et₃N) then via a second flash chromatography (1% to 10% MeOH in CHCl₃) to give material that was 80% pure. This material was treated with acetic acid (0.20 mmol, 0.012 mL), DIEA (0.63 mmol, 0.11 mL), HATU (0.24 mmol, 90 mg) and DCM (2 mL) at room temperature for 30 min. The reaction mixture was concentrated and partitioned between EtOAc and sat. NaHCO₃. The organic layer was separated, dried over Na₂SO₄ and concentrated. Purification via flash chromatography (50% to 100% EtOAc in hexanes) gave Compound I-8 (54 mg, 16%).

¹H NMR (400 MHz, CDCl₃) δ 4.12 (t, J=6.3 Hz, 2H), 3.32-3.23 (m, 4H), 3.23-3.10 (m, 8H), 2.53 (t, J=7.5 Hz, 2H), 2.38 (s, 6H), 2.27 (t, J=7.6 Hz, 4H), 2.07-1.78 (m, 5H), 1.69-1.42 (m, 4H), 1.57-1.43 (m, 12H), 1.36-1.20 (m, 77H), 0.94-0.78 (m, 12H). m/z calcd. for Chemical Formula: C₆₆H₁₃₂N₄O₄=1045.0. Found [M+H]⁺=1046.2.

### Synthetic Example 9

### 1,19-bis(didecylamino)-1,19-dioxononadecan-10-yl 5-(dimethylamino)pentanoate (Compound I-9)

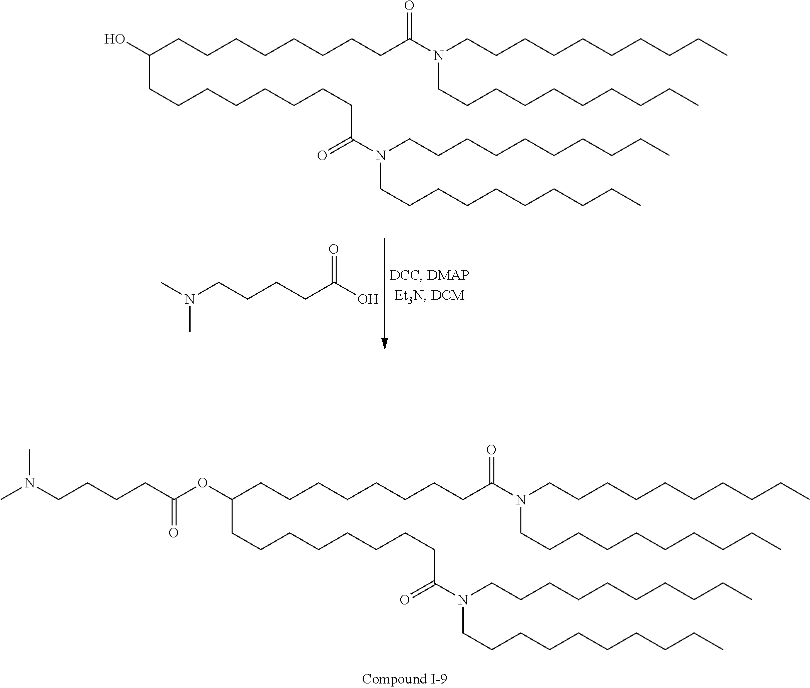

Synthesis of 1,19-bis(didecylamino)-1,19-dioxononadecan-10-yl 5-(dimethylamino)pentanoate (Compound I-9)

A mixture of N¹,N¹,N¹⁹,N¹⁹-tetrakis(decyl)-10-hydroxynonadecanediamide (1.0 eq), 5-(dimethylamino)pentanoic acid (1.05 eq), DCC (1.2 eq), 4-dimethylaminopyridine (1.0 eq), and triethylamine (4.0 eq) in DCM (0.11M) was stirred at room temperature overnight. Purification via flash chromatography (5% to 100% EtOAc in hexanes with 1% Et₃N) gave the desired compound. Yield (55 mg, 48%).

¹H NMR (400 MHz, CDCl₃) δ 4.85 (p, J=6.2 Hz, 1H), 3.32-3.23 (m, 4H), 3.23-3.14 (m, 4H), 2.35-2.22 (m, 14H), 1.98-1.69 (m, 1H), 1.70-1.56 (m, 6H), 1.53-1.44 (m, 15H), 1.39-1.07 (m, 79H), 0.96-0.77 (m, 12H). m/z calcd. for Chemical Formula: C₆₆H₁₃₁N₃O₄=1030.0. Found [M+H]⁺=1031.2.

### Synthetic Example 10

### 1,19-bis(didecylamino)-1,19-dioxononadecan-10-yl 4-(dimethylamino)butanoate (Compound I-10)

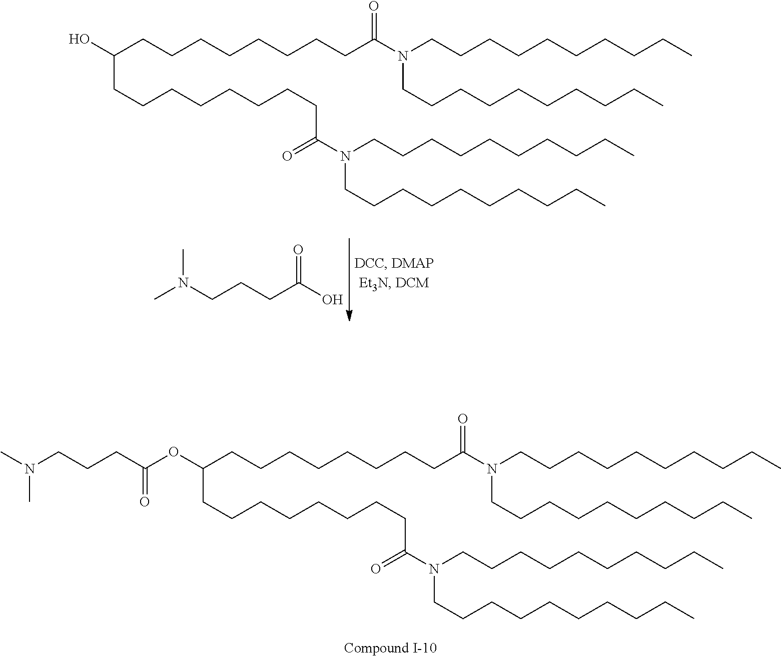

### Synthesis of 1,19-bis(didecylamino)-1,19-dioxononadecan-10-yl 4-(dimethylamino)butanoate (Compound I-10)

1,19-Bis(didecylamino)-1,19-dioxononadecan-10-yl 4-(dimethylamino)butanoate was prepared from N¹,N¹,N¹⁹,N¹⁹-tetrakis(decyl)-10-hydroxynonadecanediamide and 4-(dimethylamino)butanoic acid hydrochloride according to the general procedures of Synthetic Example 9. Yield (29 mg, 26%).

¹H NMR (400 MHz, CDCl₃) δ 4.85 (p, J=6.2 Hz, 1H), 3.32-3.23 (m, 4H), 3.23-3.14 (m, 4H), 2.76-2.42 (m, 7H), 2.42-2.33 (m, 1H), 2.30-2.22 (m, 4H), 2.04-1.88 (m, 2H), 1.68-1.43 (m, 17H), 1.36-1.14 (m, 78H), 0.95-0.82 (m, 12H). m/z calcd. for Chemical Formula: C₆₅H₁₂₉N₃O₄=1016.0. Found [M+H]⁺=1017.0.

### Synthetic Example 11

### 1,19-bis(didecylamino)-1,19-dioxononadecan-10-yl 3-(dimethylamino)propanoate (Compound I-11)

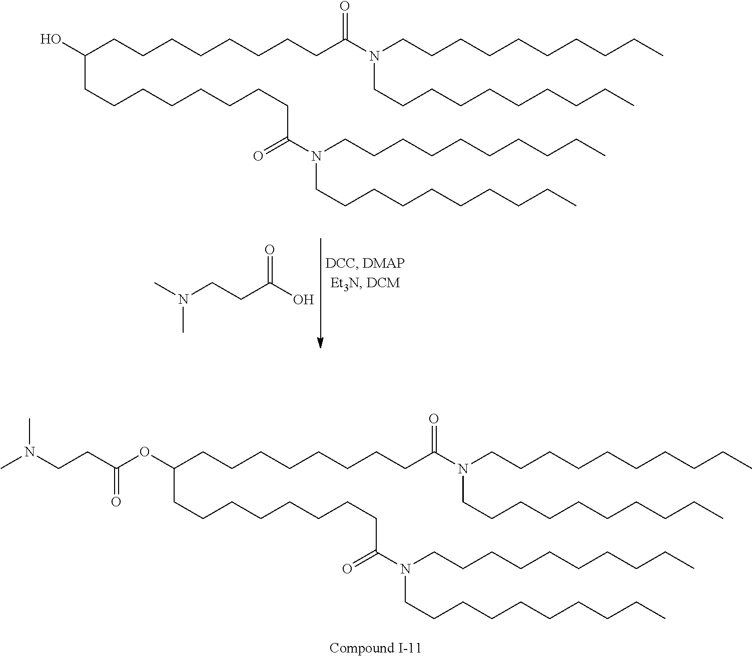

### Synthesis of 1,19-bis(didecylamino)-1,19-dioxononadecan-10-yl 3-(dimethylamino)propanoate (Compound I-11)

1,19-Bis(didecylamino)-1,19-dioxononadecan-10-yl 3-(dimethylamino)propanoate was prepared from N¹,N¹,N¹⁹,N¹⁹-tetrakis(decyl)-10-hydroxynonadecanediamide and 3-(dimethylamino)propanoic acid hydrochloride according to the general procedures of Synthetic Example 9. Yield (40 mg, 36%).

¹H NMR (400 MHz, CDCl₃) δ 4.86 (p, J=6.2 Hz, 1H), 3.32-3.23 (m, 4H), 3.23-3.15 (m, 4H), 2.67-2.54 (m, 2H), 2.49-2.39 (m, 2H), 2.33-2.17 (m, 10H), 1.67-1.58 (d, 6H), 1.57-1.44 (m, 13H), 1.38-1.07 (m, 78H), 0.97-0.78 (m, 12H). m/z calcd. for Chemical Formula: C₆₄H₁₂₇N₃O₄=1002.0. Found [M+H]⁺=1003.2.

### Synthetic Example 12

### N¹,N¹,N¹⁹,N¹⁹-tetrakis(decyl)-10-(n-decyl-5-(dimethylamino)-2-fluoropentanamido)nonadecanediamide (Compound I-12)

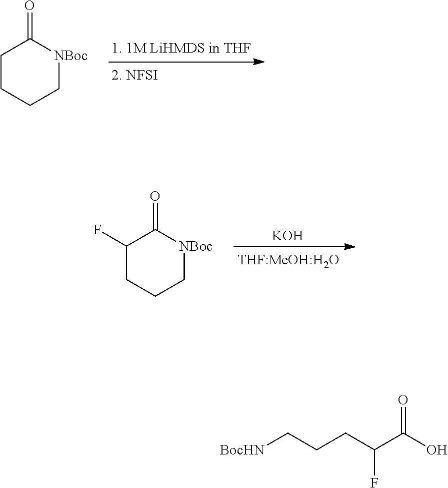 [SMILES: CC(C)(C)OC(=O)N1CCCCC1=O ; CC(C)(C)OC(=O)N1CCCC(F)C1=O ; "KOH" ; CC(C)(C)OC(=O)NCCCC(F)C(=O)O]

### Synthesis of tert-butyl 3-fluoro-2-oxopiperidine-1-carboxylate

To a solution of tert-butyl 2-oxopiperidine-1-carboxylate (5.0 mmol, 1.0 g) in THF (8 mL) was added 1M LiHMDS in THF (5.25 mmol, 5.3 mL) under argon atmosphere at −78° C. The reaction mixture was stirred at −78° C. for 1 h. NFSI (5.25 mmol, 1.66 g) was then added, and the reaction mixture was allowed to warm to −40° C. over 2 h. The reaction was quenched with saturated NaHCO₃ and extracted with EtOAc. The organic layer was separated, dried over Na₂SO₄ and concentrated. Purification via flash chromatography (5% to 40% EtOAc in hexanes) gave tert-butyl 3-fluoro-2-oxopiperidine-1-carboxylate (730 mg, 68%).

### Synthesis of 5-((tert-butoxycarbonyl)amino)-2-fluoropentanoic acid

A mixture of tert-butyl 3-fluoro-2-oxopiperidine-1-carboxylate (0.9 mmol, 200 mg) and potassium hydroxide (1.2 mmol, 67 mg) in MeOH:THF:H₂O (1 mL:1 mL:1 mL) was stirred at room temperature overnight. The reaction mixture was quenched with 1M HCl and extracted with EtOAc.

The organic layer was separated, dried over Na₂SO₄ and concentrated to give 5-((tert-butoxycarbonyl)amino)-2-fluoropentanoic acid (166 mg, 79%) which was used in the next step without further purification.

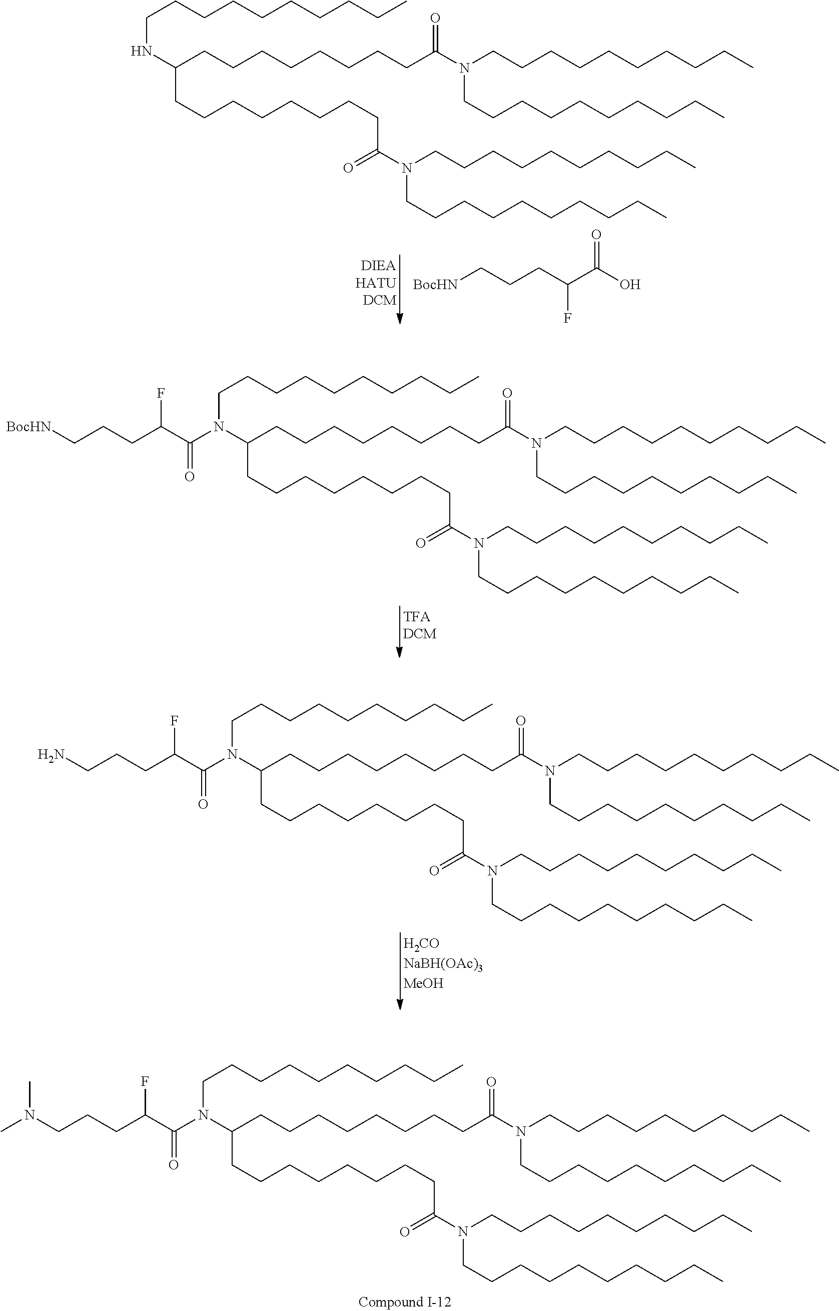

### Synthesis of N¹,N¹,N⁹,N⁹-tetrakis(decyl)-10-(N-decyl-5-(dimethylamino)-2-fluoropentanamido) nonadecanediamide (Compound I-12)

Compound I-12 was prepared from 10-(5-amino-N-decyl-2-fluoropentanamido)-N¹,N¹,N¹⁹,N¹⁹-tetrakis(decyl)nonadecanediamide according to according to the general procedures of Synthetic Example 3. A second purification via flash chromatography (2% to 10% MeOH in DCM) gave the desired product. Yield (40 mg, 26%).

¹H NMR (400 MHz, CDCl₃) δ 5.25-4.98 (m, 1H), 3.69-3.57 (m, 1H), 3.33-2.93 (m, 4H), 3.23-3.15 (m, 4H), 3.14-2.95 (m, 2H), 2.78-2.34 (m, 8H), 2.32-2.19 (m, 4H), 2.07-1.71 (m, 4H), 1.70-1.38 (m, 20H), 136-1.06 (m, 91H), 0.98-0.78 (m, 15H). m/z calcd. for Chemical Formula: C₇₆H₁₅FN₄O₃=1187.2. Found [M+H]⁺=1188.4.

### Synthetic Example 13

### N¹-butyl-N¹⁹,N¹⁹-didecyl-10-(n-decyl-3-(dimethylamino)propanamido)-N-(heptadecan-9-yl)nonadecanediamide (Compound I-17)

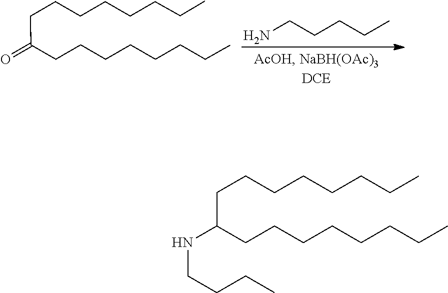

### Synthesis of N-butylheptadecan-9-amine

A mixture of heptadecan-9-one (5.9 mmol, 1.5 g), butan-1-amine (8.8 mmol, 0.87 mL), acetic acid (8.8 mmol, 0.51 mL), and sodium triacetoxyborohydride (23.6 mmol, 5.0 g) in dichloroethane (30 mL) was stirred at room temperature overnight. An additional portion of sodium triacetoxyborohydride (9.4 mmol, 2.0 g) was added and the reaction mixture was stirred for another 24 h. The reaction mixture was concentrated, and the crude was partitioned between EtOAc and sat. NaHCO₃. The organic layer was separated, dried over Na₂SO₄ and concentrated. Purification via flash chromatography (1% to 10% MeOH in CHCl₃) gave the desired product (1.29 g, 70%).

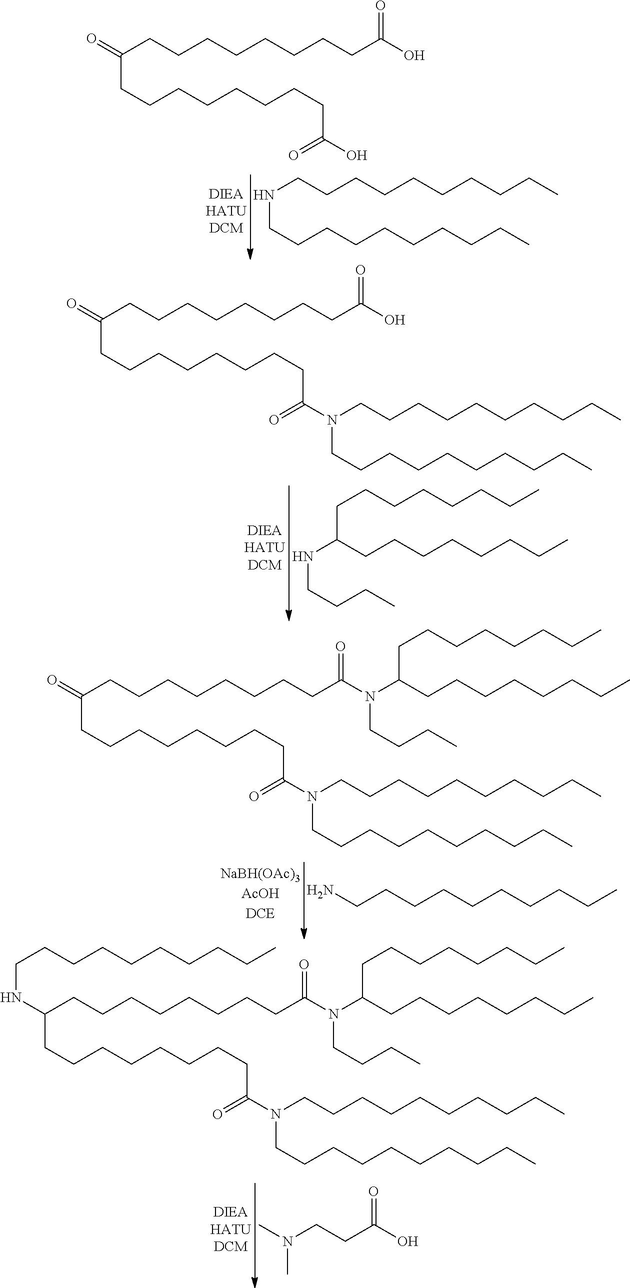

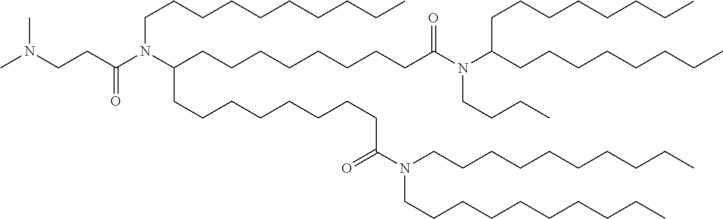 [SMILES: CCCCCCCCCCN(CCCCCCCCCC)C(=O)CCCCCCCCC(CCCCCCCCC(=O)N(CCCC)C(CCCCCCCC)CCCCCCCC)N(CCCCCCCCCC)C(=O)CCN(C)C]

### Synthesis of 19-(didecylamino)-10,19-dioxononadecanoic acid

To a mixture of commercially available 10-oxononadecanedioic acid (5.8 mmol, 2.0 g), didecylamine (5.8 mmol, 1.7 g), and DIPEA (17.5 mmol, 3.05 mL) in DCM (29 mL) was added HATU (7.6 mmol, 2.9 g) and the reaction mixture was stirred at room temperature for 50 min. The reaction mixture was concentrated, and the crude was partitioned between EtOAc and 1M HCl. The organic layer was separated, dried over Na₂SO₄ and concentrated. The crude material was purified via automated flash chromatography (1% to 10% MeOH in DCM). The isolated product was triturated in hexanes and filtered. The filtrate was subjected to another purification via automated flash chromatography (10% to 70% EtOAc in hexanes) to give desired product (500 mg, 14%).

### Synthesis of N¹-butyl-N¹⁹,N¹⁹-didecyl-N-(heptadecan-9-yl)-10-oxononadecanediamide

A mixture of 19-(didecylamino)-10,19-dioxononadecanoic acid (1.3 mmol, 0.80 g), N-butylheptadecan-9-amine (1.3 mmol, 400 mg), DIPEA (3.9 mmol, 0.67 mL), and HATU (1.7 mmol, 640 mg) in DCM (6 mL) was stirred at room temperature for 2 h. The reaction mixture was concentrated, and the crude was partitioned between EtOAc and sat. NaHCO₃. The organic layer was separated, dried over Na₂SO₄ and concentrated. Purification via flash chromatography (5% to 70% EtOAc in hexanes) gave N¹-butyl-N¹⁹,N¹⁹-didecyl-N¹-(heptadecan-9-yl)-10-oxononadecanediamide (1.07 g, 91%).

### Synthesis of N¹-butyl-N¹⁹,N¹⁹-didecyl-10-(decylamino)-N¹-(heptadecan-9-yl)nonadecanediamide

N¹-butyl-N¹⁹,N¹⁹-didecyl-10-(decylamino)-N¹-(heptadecan-9-yl)nonadecanediamide was prepared from N¹-butyl-N¹⁹,N¹⁹-didecyl-N¹-(heptadecan-9-yl)-10-oxononadecanediamide according to the procedure detailed in Synthetic Example 1. Yield (570 mg, 87%).

### Synthesis of Compound I-17

Compound I-17 was prepared from N¹-butyl-N¹⁹,N¹⁹-didecyl-10-(decylamino)-N¹-(heptadecan-9-yl)nonadecanediamide and 3-(dimethylamino)propanoic acid according to the procedure described in Synthetic Example 1. Yield (67 mg, 51%).

¹H NMR (400 MHz, CDCl₃) δ 4.55-4.28 (m, 1H), 3.67-3.56 (m, 1H), 3.32-3.23 (m, 2H), 3.23-3.15 (m, 2H), 3.10-2.96 (m, 4H), 2.72-2.60 (m, 2H), 2.54-2.40 (m, 2H), 2.32-2.21 (m, 10H), 1.71-1.08 (m, 109H), 1.00-0.83 (m, 18H). m/z calcd. for Chemical Formula: C₇₅H₁₅₀N₄O₃=1155.2. Found [M+H]⁺=1156.4.

### Synthetic Example 14

### 1,13-bis(didecylamino)-1,13-dioxotridecan-7-yl 3-(dimethylamino)propanoate (Compound I-19)

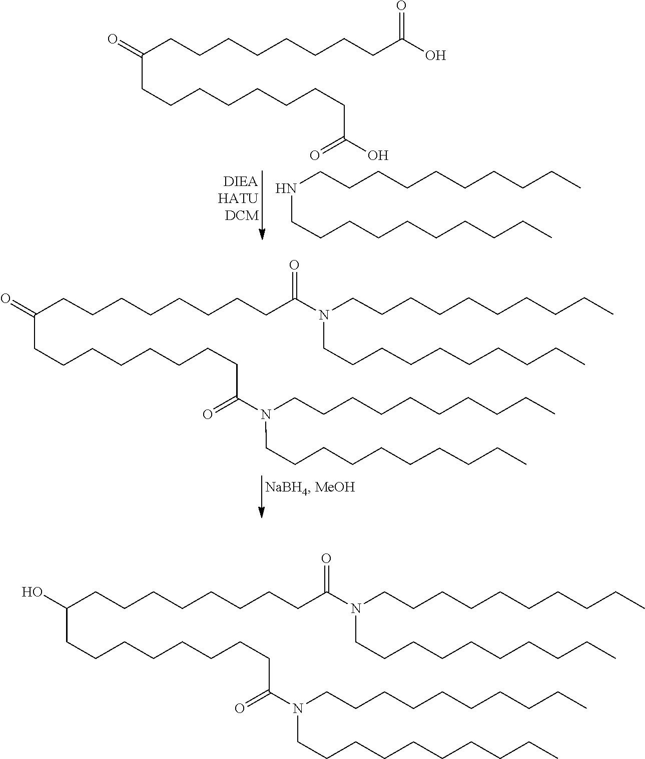

### Synthesis of Intermediate N¹,N¹,N¹⁹,N¹⁹-tetrakis(decyl)-10-oxononadecanediamide

A mixture of 10-oxononadecanedioic acid (5.8 mmol, 2.0 g), dodecylamine (12.8 mmol, 3.8 g), DIPEA (35 mmol, 6.1 mL) and HATU (15.2 mmol, 5.8 g) in DCM (29 mL) was stirred at room temperature overnight. The reaction mixture was concentrated and then partitioned between EtOAc and sat. NaHCO₃. The organic layer was separated, dried over Na₂SO₄ and concentrated. Purification via flash chromatography (5% to 50% EtOAc in hexanes) gave desired product (4.3 g, 81%).

### Synthesis of Intermediate N¹,N¹,N¹⁹,N¹⁹-tetrakis(decyl)-10-hydroxynonadecanediamide

To a solution of N¹,N¹,N⁹,N¹⁹-tetrakis(decyl)-10-oxononadecanediamide (0.56 mmol, 500 mg) in methanol (10 mL) was added sodium borohydride (0.56 mmol, 21 mg). The reaction mixture was stirred at room temperature overnight. The reaction was quenched with sat. NaHCO₃ and extracted with EtOAc. The organic layer was dried over Na₂SO₄ and concentrated to give desired product (470 m. 94%) which was used in the subsequent step without further purification.

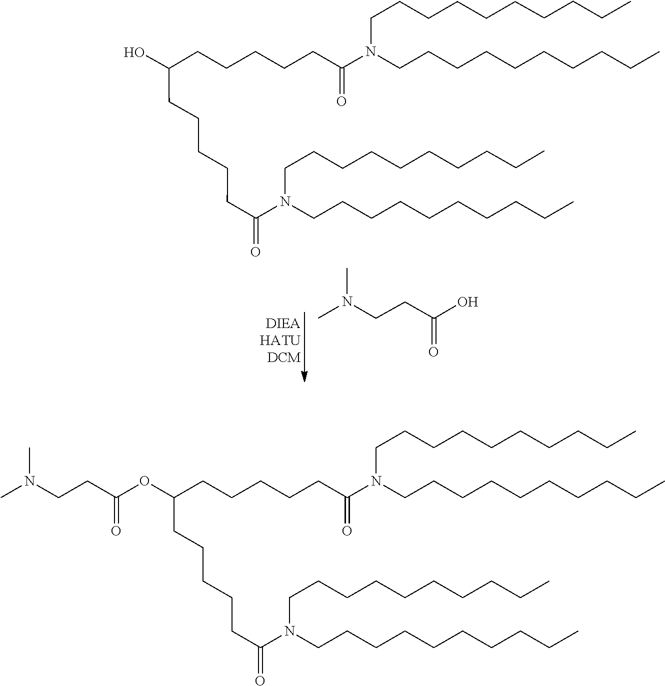

### Synthesis of N¹,N¹,N¹³,N¹³-tetrakis(decyl)-7-hydroxytridecanediamiide

N¹,N¹,N¹³,N¹³-tetrakis(decyl)-7-hydroxytridecanediamide was synthesized from commercially available 7-oxotridecanedioic acid according to the procedure for N¹,N¹,N¹⁹,N¹⁹-tetrakis(decyl)-10-hydroxynonadecanediamide. Yield (1.0 g, quantitative).

### Synthesis of Compound I-19

Compound I-19 was prepared from N¹,N¹,N¹³,N¹³-tetrakis(decyl)-7-hydroxytridecanediamide and 3-(dimethylamino)propanoic acid according to the procedure for Compound I-11. A second purification via flash chromatography was required (1% to 10% MeOH in CHCl₃). Yield (95 mg, 57%).

¹H NMR (400 MHz, CDCl₃) δ 4.94-4.82 (m, 1H), 3.34-3.25 (m, 4H), 3.25-3.16 (m, 4H), 2.66-2.58 (m, 2H), 2.51-2.43 (m, 2H), 2.32-2.23 (m, 10H), 1.72-1.46 (m, 20H), 1.39-1.23 (m, 64H), 0.95-0.86 (m, 12H). m/z calcd. for Chemical Formula: C₅₈H₁₁₅N₃O₄=917.9. Found [M+H]⁺=919.2.

### Synthetic Example 15

### 1,13-bis(didecylamino)-1,13-dioxotridecan-7-yl 4-(dimethylamino)butanoate (Compound I-18)

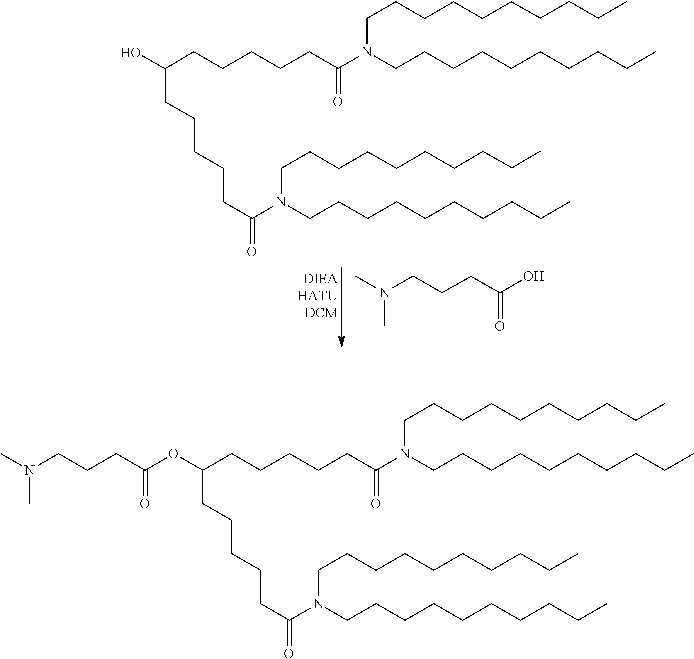

### Synthesis of Compound I-18

Compound I-18 was prepared from N¹,N¹,N¹³,N¹³-tetrakis(decyl)-7-hydroxytridecanediamide and 4-(dimethylamino)butanoic acid according to the procedure for Compound I-11. Yield (93 mg, 54%).

¹H NMR (400 MHz, CDCl₃) δ 4.91-4.80 (m, 1H), 3.31-3.24 (m, 4H), 3.23-3.13 (m, 4H), 2.35-2.18 (m, 45H), 1.83-1.71 (m, 2H), 1.70-1.40 (m, 22H), 1.40-1.08 (m, 65H), 0.92-0.83 (m, 12H). m/z calcd. for Chemical Formula: C₅₉H₁₁₇N₃O₄=931.9. Found [M+H]⁺=933.2.

### Synthetic Example 16

### 3-(pyrrolidin-1-yl)propyl bis(10-(n-dodecyloctanamido)decyl)carbamate (Compound I-20)

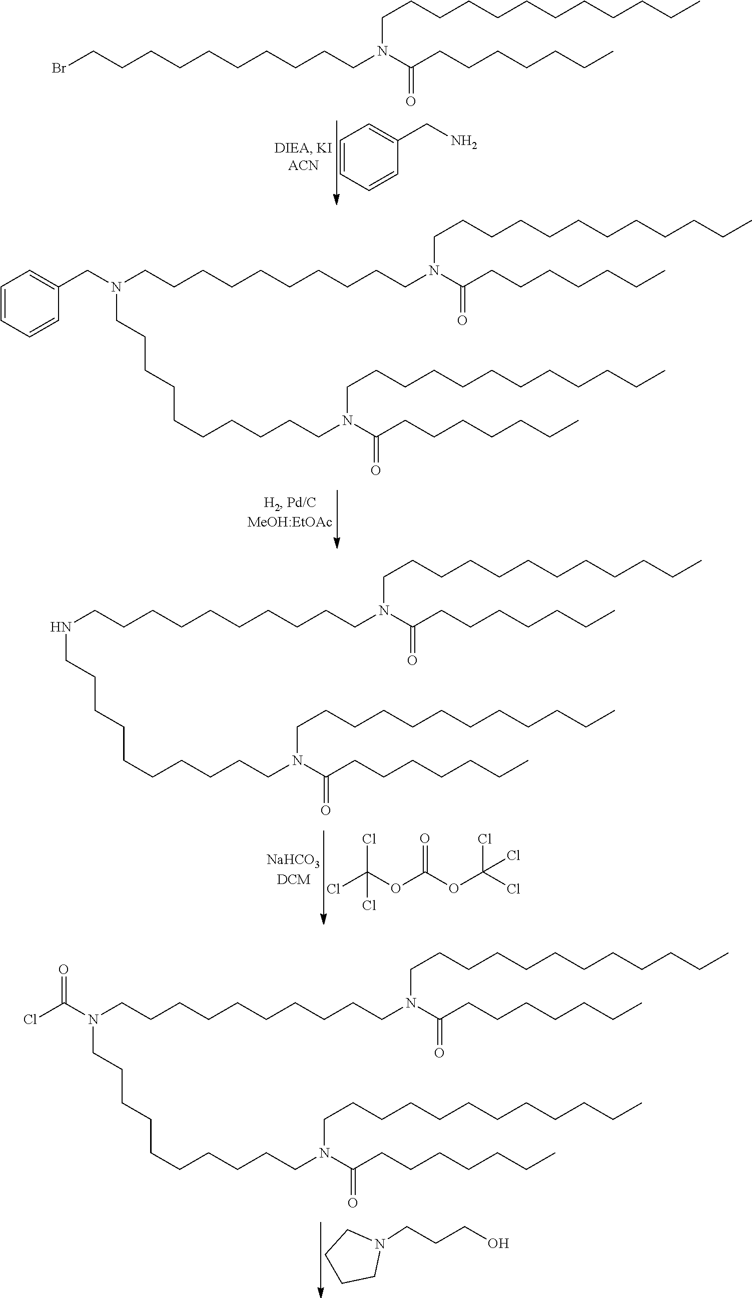

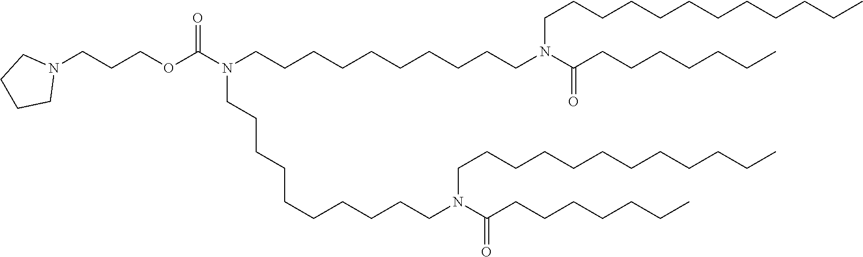 [SMILES: CCCCCCCCCCCCN(CCCCCCCCCCN(CCCCCCCCCCN(CCCCCCCCCCCC)C(=O)CCCCCCC)C(=O)OCCCN1CCCC1)C(=O)CCCCCCC]

### Synthesis of N-(10-bromodecyl)-N-dodecyloctanamide

N-(10-bromodecyl)-N-dodecyloctanamide was prepared starting from 10-amiinodecan-1-ol and 1-bromododecane according to the procedure described in Synthetic Example 3. Yield (612 mg, 8.9% over 3 steps).

### Synthesis of N,N′-((benzylazanediyl)bis(decane-10,1-diyl))bis(N-dodecyloctanamide)

N,N′-((benzylazanediyl)bis(decane-10,1-diyl))bis(N-dodecyloctanamide) was prepared from N-(10-bromodecyl)-N-dodecyloctanamide according to the procedure described in Synthetic Example 2. Yield (289 mg, 49%).

### Synthesis of N,N′-(azanediylbis(decane-10,1-diyl))bis(N-dodecyloctanamide)

N,N′-(azanediylbis(decane-10,1-diyl))bis(N-dodecyloctanamide) was prepared from N,N′-((benzylazanediyl)bis(decane-10,1-diyl))bis(N-dodecyloctanamide) according to the procedure described in Synthetic Example 2. Yield (253 mg, 98%).

### Synthesis of Compound I-20

Compound I-20 was prepared in two steps from starting materials according to the procedure described in Synthetic Example 6. Purification via flash chromatography (5% to 100% EtOAc in hexanes with 1% Et₃N) followed by a second purification via flash chromatography (c18 column, 80% to 100% MeOH in water with 0.1% TFA) to give desired product (40 mg, 24% over 2 steps).

¹H NMR (400 MHz, CDCl₃) δ 4.11 (t, J=6.4 Hz, 2H), 3.32-3.24 (m, 4H), 3.23-3.10 (m, 6H), 2.57-2.49 (m, 6H), 2.31-2.22 (m, 4H), 1.92-1.75 (m, 6H), 1.69-1.12 (m, 98H), 0.92-0.83 (m, 12H). m/z calcd. for Chemical Formula: C₆₈H₁₃₄N₄O₄=1071.0. Found [M+H]⁺=1072.3.

### Synthetic Example 17

### 1,16-bis(didecylamino)-1,16-dioxohexadecan-6-yl 4-(dimethylamino)butanoate (Compound I-21)

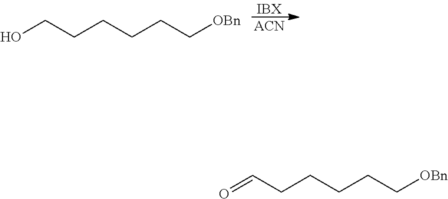 [SMILES: OCCCCCCOCc1ccccc1 ; O=CCCCCCOCc1ccccc1]

### Synthesis of 6-(benzyloxy)hexanal

A mixture of commercially available 6-(benzyloxy)hexan-1-ol (24 mmol, 5.0 g) and 2-iodoxybenzoic acid (72 mmol, 45 g, 45% w/w) in acetonitrile was heated at 80° C. for 3 h. The reaction mixture was cooled to room temperature, diluted with hexanes, and filtered. The filtrate was concentrated and the crude material was purified via flash chromatography (0% to 30% EtOAc in hexanes) to give desired product (2.8 g, 57%).

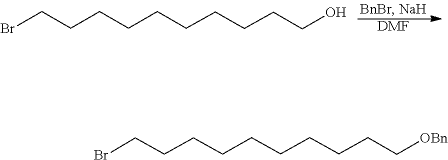 [SMILES: OCCCCCCCCCCBr ; BrCCCCCCCCCCOCc1ccccc1]

### Synthesis of (((10-bromodecyl)oxy)methyl)benzene

To a cooled (5° C.) mixture of commercially available 10-bromodecan-1-ol (42.2 mmol, 10 g) and benzyl bromide (126 mmol, 15 mL) in anhydrous THF (85 mL) was added 60% w/w sodium hydride (148 mmol, 5.9 g). The reaction mixture was warmed to room temperature and stirred for 19 h. The reaction was quenched with sat. NaHCO₃ and extracted with dichloromethane. The organic layer was separated, dried over Na₂SO₄ and concentrated. Purification via flash chromatography (0% to 20% EtOAc in hexanes) gave desired product (10 g, 72%).

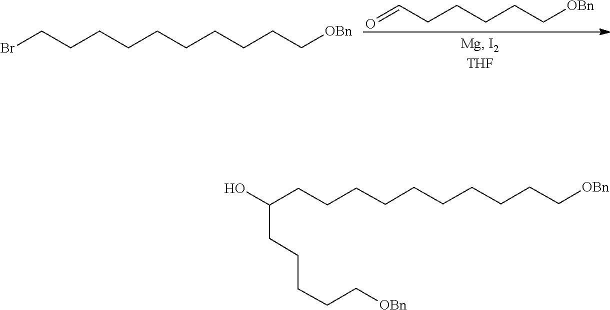

### Synthesis of 1,16-bis(benzyloxy)hexadecan-6-ol

To starting material (7.4 mmol, 4.9 g) in anhydrous THF (30 mL) was added magnesium (45 mmol, 1.1 g) and iodine (0.74 mmol, 94 mg). The reaction mixture was heated at 60° C. for 30 min then cooled to room temperature. 6-(benzyloxy)hexanal (7.4 mmol, 1.5 g) in anhydrous THF (9 mL) was added dropwise and the reaction mixture was heated at 60° C. for 3 hours then at room temperature for 19 h. The reaction was quenched with sat. NaHCO₃ and extracted with dichloromethane. The organic layer was separated, dried over Na₂SO₄ and concentrated. Purification via flash chromatography (0% to 50% EtOAc in hexanes) gave desired product (2.9 g, 85%).

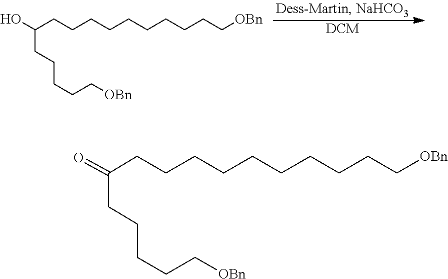 [SMILES: OC(CCCCCCCCCCOCc1ccccc1)CCCCCOCc1ccccc1 ; O=C(CCCCCCCCCCOCc1ccccc1)CCCCCOCc1ccccc1]

### Synthesis of 1,16-bis(benzyloxy)hexadecan-6-one

A mixture of 1,16-bis(benzyloxy)hexadecan-6-ol (6.3 mmol, 2.9 g), sodium bicarbonate (38 mmol, 3.2 g) and Dess-Martin periodinane (12.5 mmol, 5.3 g) in dichloromethane (21 mL) was stirred at room temperature for 3 hours. The reaction mixture was cooled to room temperature and filtered. The filtrate was purified via flash chromatography (0% to 30% EtOAc in hexanes) to give desired product 2.6, 90%).

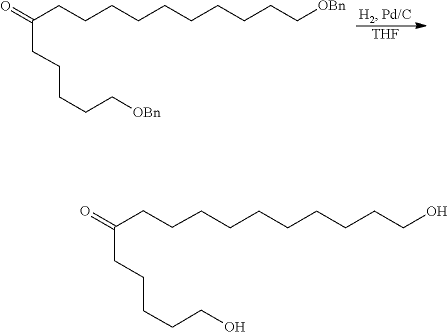 [SMILES: O=C(CCCCCCCCCCOCc1ccccc1)CCCCCOCc1ccccc1 ; O=C(CCCCCO)CCCCCCCCCCO]

### Synthesis of 1,16-dihydroxyhexadecan-6-one

A mixture of 1,16-bis(benzyloxy)hexadecan-6-one (4.9 mmol, 2.2 g) and Pd/C (10% w/w, 0.97 mmol, 1.0 g) in THF (49 mL) was stirred at room temperature under hydrogen (balloon pressure) for 2 h. The reaction mixture was filtered over a pad of diatomaceous earth (e.g., Celite®) and the filtrate concentrated to give 1,16-dihydroxyhexadecan-6-one (1.34 g, quantitative).

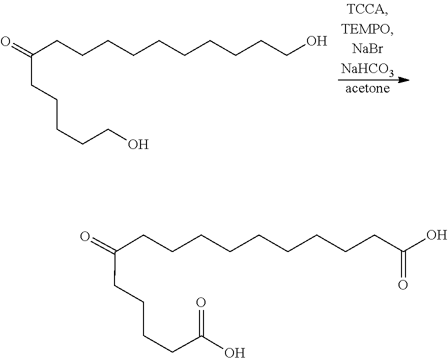 [SMILES: O=C(CCCCCO)CCCCCCCCCCO ; O=C(O)CCCCCCCCCC(=O)CCCCC(=O)O]

### Synthesis of 6-oxohexadecanedioic acid

A mixture of 1,16-dihydroxyhexadecan-6-one (5.25 mmol, 1.43 g), trichloroisocyanuric acid (21 mmol, 4.9 g), TEMPO (0.21 mmol, 33 mg), saturated sodium bicarbonate (16 mL), and sodium bromide (2.1 mmol, 216 mg) in acetone (53 mL) was stirred at room temperature for 19 hours. The reaction mixture was quenched with i-PrOH (12 mL), stirred at room temperature for 30 min then filtered. The filtrate was concentrated and to the crude material was added 1M NaOH. The aqueous layer was washed with DCM, acidified with 1M HCl and extracted with EtOAc. The EtOAc layer was separated, dried over Na₂SO₄, and concentrated to give 6-oxohexadecanedioic acid (1.4 g, 89%) which was used without further purification.

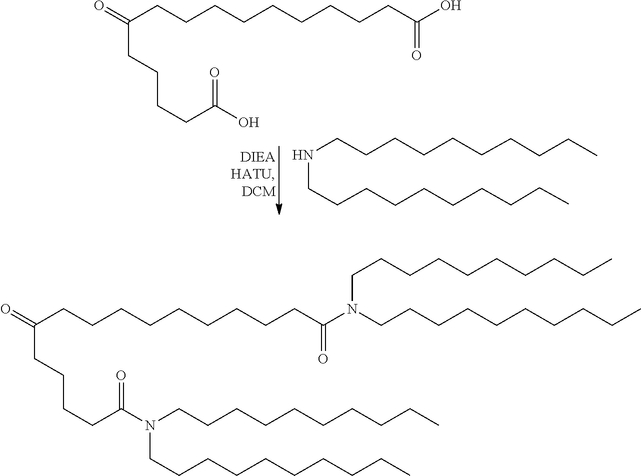 [SMILES: O=C(O)CCCCCCCCCC(=O)CCCCC(=O)O ; CCCCCCCCCCNCCCCCCCCCC ; CCCCCCCCCCN(CCCCCCCCCC)C(=O)CCCCCCCCCC(=O)CCCCC(=O)N(CCCCCCCCCC)CCCCCCCCCC]

### Synthesis of N¹,N¹,N¹⁶,N¹⁶-tetrakis(decyl)-6-oxohexadecanediamide

A mixture of 6-oxohexadecanedioic acid (1.0 mmol, 0.3 g), didecylamine (2.1 mmol, 624 mg), DIPEA (6.0 mmol, 1.0 mL), and HATU (2.6 mmol, 987 mg) in dichloromethane (10 mL) was stirred at room temperature for 19 hours. The reaction mixture was concentrated, and the crude was partitioned between EtOAc and sat. NaHCO₃. The organic layer was separated, dried over Na₂SO₄ and concentrated. Purification via flash chromatography (0% to 30% EtOAc in hexanes) gave N¹,N¹,N⁶,N⁶-tetrakis(decyl)-6-oxohexadecanediamide (635 mg, 74%).

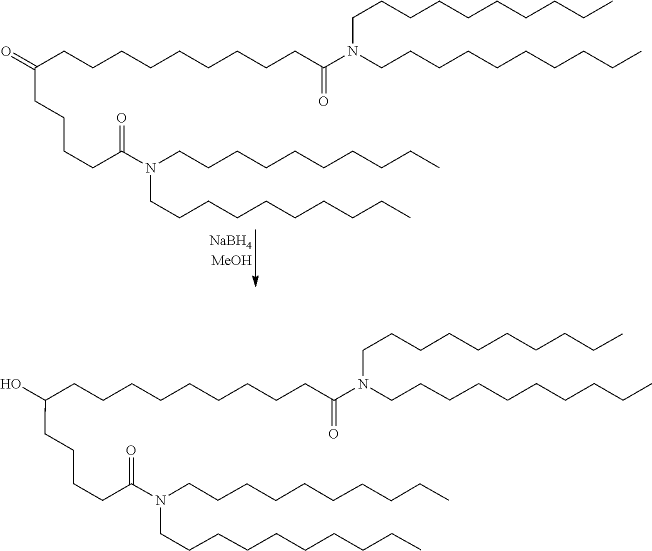 [SMILES: CCCCCCCCCCN(CCCCCCCCCC)C(=O)CCCCCCCCCC(=O)CCCCC(=O)N(CCCCCCCCCC)CCCCCCCCCC ; CCCCCCCCCCN(CCCCCCCCCC)C(=O)CCCCCCCCCC(O)CCCCC(=O)N(CCCCCCCCCC)CCCCCCCCCC]

### Synthesis of N¹,N¹,N¹⁶,N¹⁶-tetrakis(decyl)-6-hydroxyhexadecanediamide

To N¹,N¹,N¹⁶,N¹⁶-tetrakis(decyl)-6-oxohexadecanediamide (0.32 mmol, 275 mg) in MeOH (10 mL) at room temperature was added sodium borohydride (1.6 mmol, 60 mg) in three portions over 3 days. Following completion of the reaction, the reaction mixture was concentrated. The crude material was partitioned saturated NaHCO₃ and EtOAc. The organic layer was separated, dried over Na₂SO₄ and concentrated to give N¹,N¹,N¹⁶,N¹⁶-tetrakis(decyl)-6-hydroxyhexadecanediamide (180 mg, 46%).

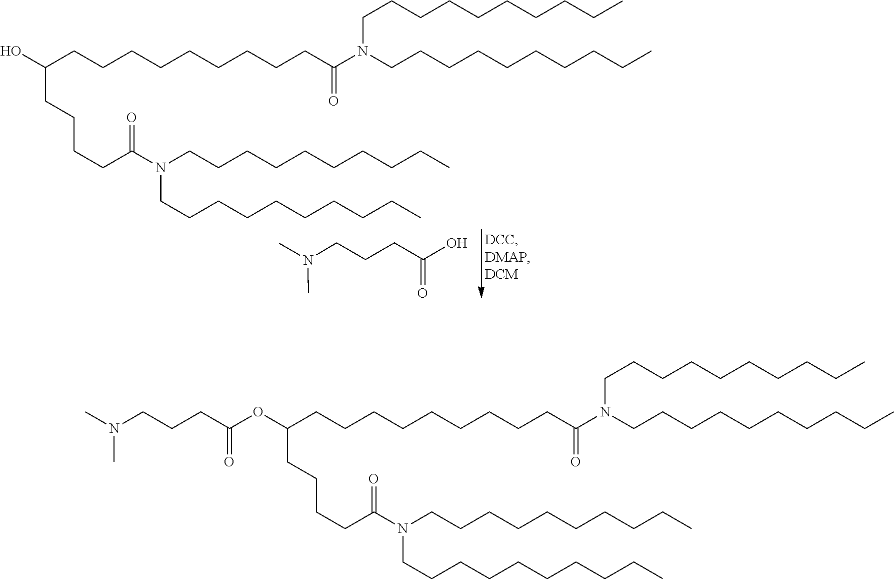

### Synthesis of Compound I-21

A mixture of N¹,N¹,N¹⁶,N¹⁶-tetrakis(decyl)-6-hydroxyhexadecanediamide (0.12 mmol, 0.10 g), 4-(dimethylamino)butanoic acid (0.14 mmol, 18 mg), DCC (0.14 mmol, 29 mg), and DMAP (0.14 mmol, 17 mg) in dichloromethane (1.2 ML) was stirred at room temperature for 19 h. Additional portions of 4-(dimethylamino)butanoic acid (0.31 mmol, 40 mg), DCC (0.29 mmol, 60 mg) and DMAP (0.29 mmol, 35 mg) were added and the reaction mixture was stirred at room temperature for another 19 h. The reaction mixture was diluted with hexanes and filtered. The filtrate was purified via flash chromatography (5% to 65% EtOAc in hexanes with 1% Et₃N) to give desired product (52 mg, 46%).

¹H NMR (400 MHz, CDCl₃) δ 4.94-4.83 (m, 1H), 3.34-3.26 (m, 4H), 3.26-3.14 (m, 4H), 2.39-2.14 (m, 14H), 1.87-1.74 (m, 2H), 1.73-1.10 (m, 91H), 0.95-0.85 (m, 12H). m/z calcd. for Chemical Formula: C₆₂H₁₂₃N₃O₄=974.0. Found [M+H]⁺=975.2.

The various embodiments described above can be combined to provide further embodiments. All the U.S. patents, U.S. patent application publications, U.S. patent applications, foreign patents, foreign patent applications and non-patent publications referred to in this specification are incorporated herein by reference, in their entirety. Aspects of the embodiments can be modified, if necessary to employ concepts of the various patents, applications, and publications to provide yet further embodiments.

These and other changes can be made to the embodiments considering the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1. A compound having a structure of Formula (I): 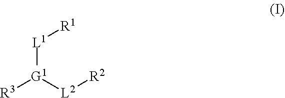
or a pharmaceutically acceptable salt, tautomer, or stereoisomer thereof, wherein:
G¹ is N or CH;
R¹ is —C(═O)N(R^{1a})R^{1b} or —NR^{1c}—C(═O)—R^{1d};
R² is —C(═O)N(R^{2a})R^{2b} or —NR^{2c}—C(═O)—R^{2d};
R^{1c} and R^{2c} are each independently hydrogen, C₁-C₁₆ alkyl, or C₂-C₁₆ alkenyl;
R^{1a} and R^{2a} are each independently C₄-C₂₄ alkyl or C₄-C₂₄ alkenyl;
R^{1b}, R^{2b}, R^{1d}, and R^{2d} is independently C₆-C₂₄ alkyl or C₆-C₂₄ alkenyl;
R^{3a} is —C(═O)—R^{3a} when G¹ is N or R³ is R^{3c}, —NR^{3b}C(═O)—R^{3a}, or —OC(═O)—R^{3a} when G¹ is CH;
R^{3a} is C₁-C₈ alkyl or C₁-C₈ alkoxy optionally substituted with one or more substituents selected from the group consisting of halo, oxo, —OH, —CN, —N(R⁴)R⁵, C₁-C₈ alkoxy, C₁-C₈ hydroxylalkoxy, C₁-C₈ alkoxyalkoxy, —C(═O)N(R⁴)R⁵, —NR⁴—C(═O)—R⁵, —N(R⁴)C(═O)OR⁵; —C(═O)OR⁶, —OC(═O)R⁶, cycloalkyl, heterocyclyl, and heteroaryl;
R^{3b} is C₁-C₁₈ alkyl optionally substituted with one or more substituents selected from the group consisting of halo, oxo, —OH, and —N(R⁴)R⁵;
R^{3c} is C₁-C₈ alkoxy optionally substituted with one or more substituents selected from the group consisting of halo, oxo, —OH, —CN, —N(R⁴)R⁵, C₁-C₈ alkoxy, C₁-C₈ hydroxylalkoxy, C₁-C₈ alkoxyalkoxy, —C(═O)N(R⁴)R⁵, —NR⁴—C(═O)—R⁵, —N(R⁴)C(═O)OR⁵; —C(═O)OR⁶, —OC(═O)R⁶, cycloalkyl, heterocyclyl, and heteroaryl;
each occurrence of R⁴ is independently hydrogen or C₁-C₈ alkyl;
each occurrence of R⁵ is independently C₁-C₈ alkyl, C₃-C₈ cycloalkyl, or 3-8 membered heterocyclyl;
R⁶ is hydrogen, C₁-C₁₂ alkyl, C₂-C₁₂ alkenyl, or aralkyl;
L¹ is C₅-C₁₂ alkylene; and
L² is C₄-C₁₂ alkylene,
wherein each alkyl, alkenyl, alkylene, alkoxy, hydroxylalkoxy, alkoxyalkoxy, cycloalkyl, heterocyclyl, heteroaryl and aralkyl is optionally substituted with one or more fluoro.

2. The compound of claim 1, wherein the compound has the following Formula (Ia): 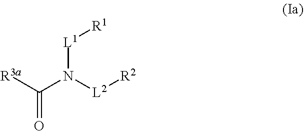
or a pharmaceutically acceptable salt, tautomer, or stereoisomer thereof.

3. The compound of claim 1, wherein the compound has the following Formula (Ib): 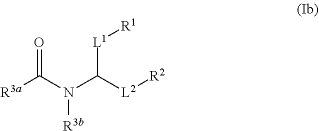
or a pharmaceutically acceptable salt, tautomer, or stereoisomer thereof.

4. The compound of claim 1, wherein the compound has the following Formula (Ic): 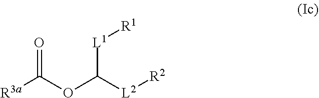
or a pharmaceutically acceptable salt, tautomer, or stereoisomer thereof.

5. The compound of claim 1, wherein the compound has the following Formula (Id): 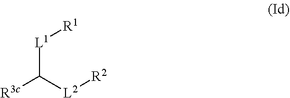
or a pharmaceutically acceptable salt, tautomer, or stereoisomer thereof.

6. The compound of claim 1, wherein R¹ is —C(═O)N(R^{1a})R^{1b}.

7. The compound of claim 6, wherein R^{1a} is C₄-C₂₄ alkyl.

8-9. (canceled)

10. The compound of claim 6, wherein R^{1a} is unbranched and unsubstituted C₄, C₈, or C₁₀ alkyl.

11. The compound of claim 6, wherein R^{1b} is unbranched and unsubstituted C₆-C₂₄ alkyl.

12. (canceled)

13. The compound of claim 6, wherein R^{1b} is unbranched and unsubstituted C₁₀ alkyl.

14. (canceled)

15. The compound of claim 6, wherein R^{1b} is branched and unsubstituted C₁₆-C₁₈ alkyl.

16. The compound of claim 1, wherein R¹ is —NR^{1c}—C(═O)—R^{1d}.

17. The compound of claim 16, wherein R^{1c} is hydrogen or unbranched and unsubstituted C₁₀ alkyl or C₁₂ alkyl.

18-19. (canceled)

20. The compound of claim 16, wherein R^{1d} is unbranched and unsubstituted C₆-C₂₄ alkyl.

21-22. (canceled)

23. The compound of claim 1, wherein R¹ has one of the following structures:

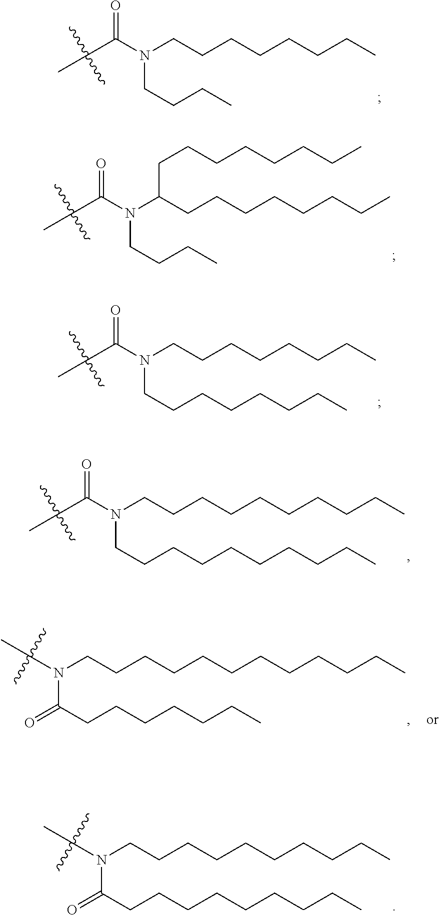

24. The compound of claim 1, wherein R² is —C(═O)N(R^{2a})R^{2b}.

25. The compound of claim 24, wherein R^{2a} is C₄-C₂₄ alkyl.

26-27. (canceled)

28. The compound of claim 24, wherein R^{2a} is unbranched and unsubstituted C₄, C₈, or C₁₀ alkyl.

29. The compound of claim 24, wherein R^{2b} is unbranched and unsubstituted C₄-C₂₄ alkyl.

30. (canceled)

31. The compound of claim 24, wherein R^{2b} is unbranched and unsubstituted C₄, C₈, or C₁₀ alkyl.

32. The compound of claim 1, wherein R² is —NR^{2c}—C(═O)—R^{2d}.

33. The compound of claim 32, wherein R^{2c} is hydrogen or unbranched and unsubstituted C₁₀ alkyl or C₁₂ alkyl.

34-35. (canceled)

36. The compound of claim 32, wherein R^{2d} is unbranched and unsubstituted C₆-C₂₄ alkyl.

37-38. (canceled)

39. The compound of claim 1, wherein R² has one of the following structures:

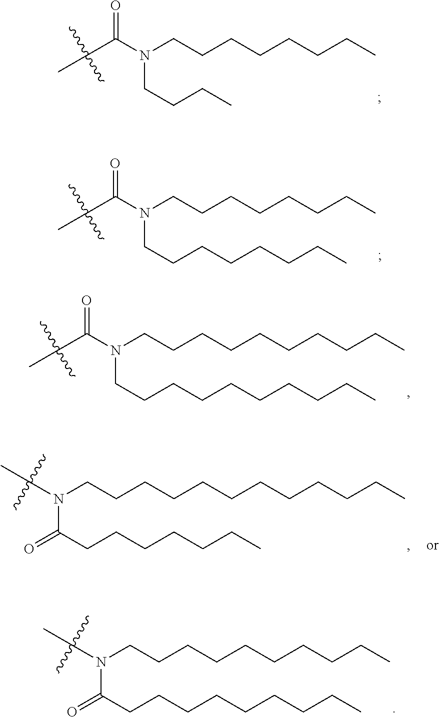 [SMILES: *C(=O)N(CCCC)CCCCCCCC ; *C(=O)N(CCCCCCCC)CCCCCCCC ; *C(=O)N(CCCCCCCCCC)CCCCCCCCCC ; *N(CCCCCCCCCCCC)C(=O)CCCCCCC ; *N(CCCCCCCCCC)C(=O)CCCCCCCCC]

40-42. (canceled)

43. The compound of claim 1, wherein R^{3a} has one of the following structures:

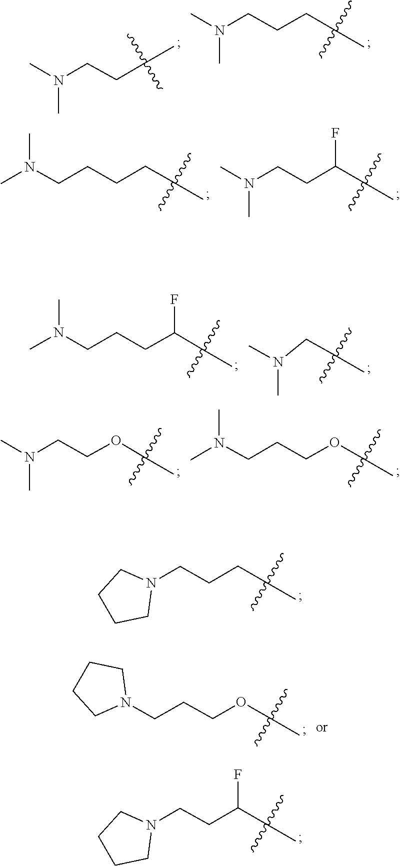

44. (canceled)

45. The compound of claim 1, wherein R^{3b} is —CH₃ or unbranched and unsubstituted C₈-C₁₀ alkyl.

46. (canceled)

47. The compound of claim 1, wherein R^{3c} has one of the following structures:

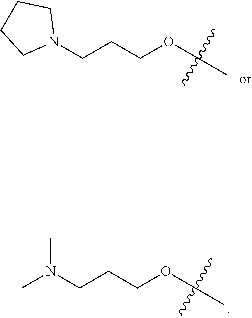

48. The compound of claim 1, wherein L¹ is C₅-C₁₀ alkylene and L² is C₄-C₁₀ alkylene.

49. The compound of claim 1, wherein L¹ is C₅, C₇, C₈, C₉, or C₁₀ alkylene.

50. (canceled)

51. The compound of claim 1, wherein L¹ and L² are both C₅ alkylene, both C₇ alkylene, both C₈ alkylene, both C₉ alkylene, or both C₁₀ alkylene.

52-56. (canceled)

57. The compound of claim 1, wherein L¹ is C₈-C₁₀ alkylene and L² is C₄-C₆ alkylene.

58. (canceled)

59. The compound of claim 1, wherein the compound has one of the following structures: 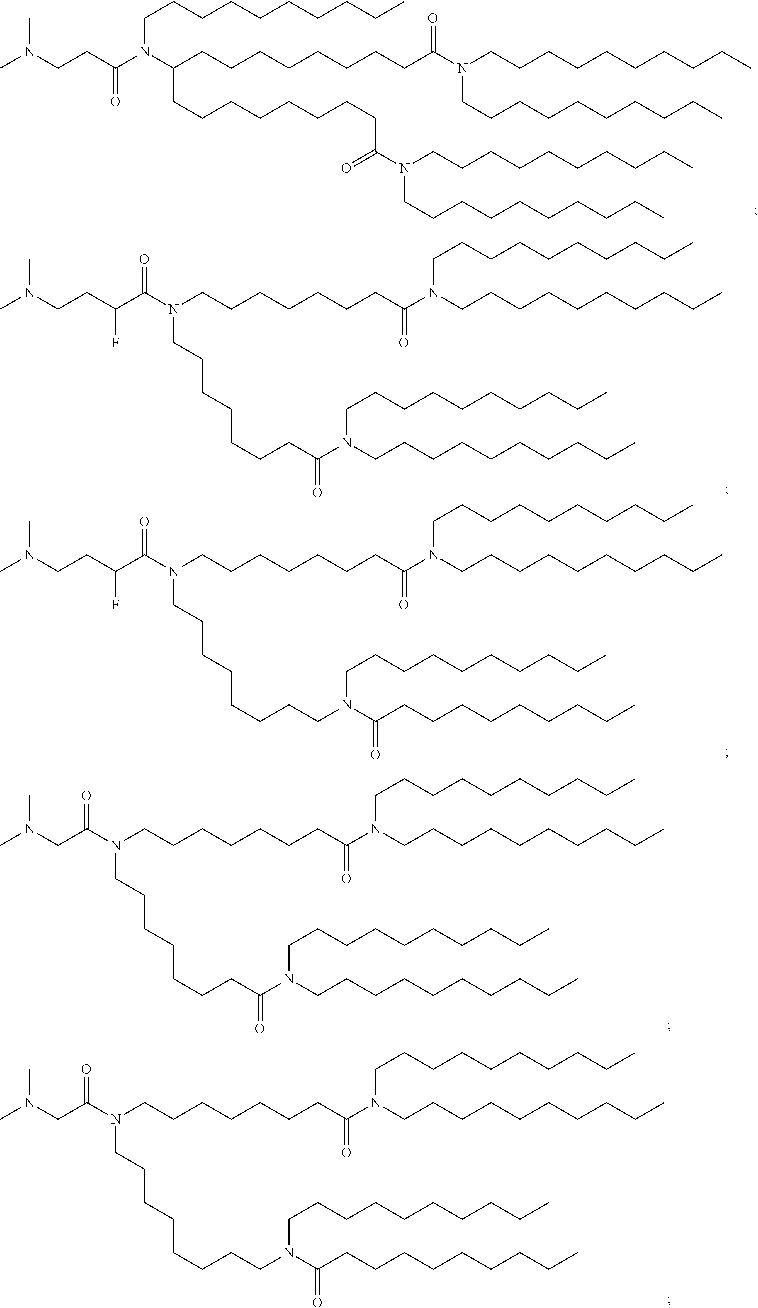 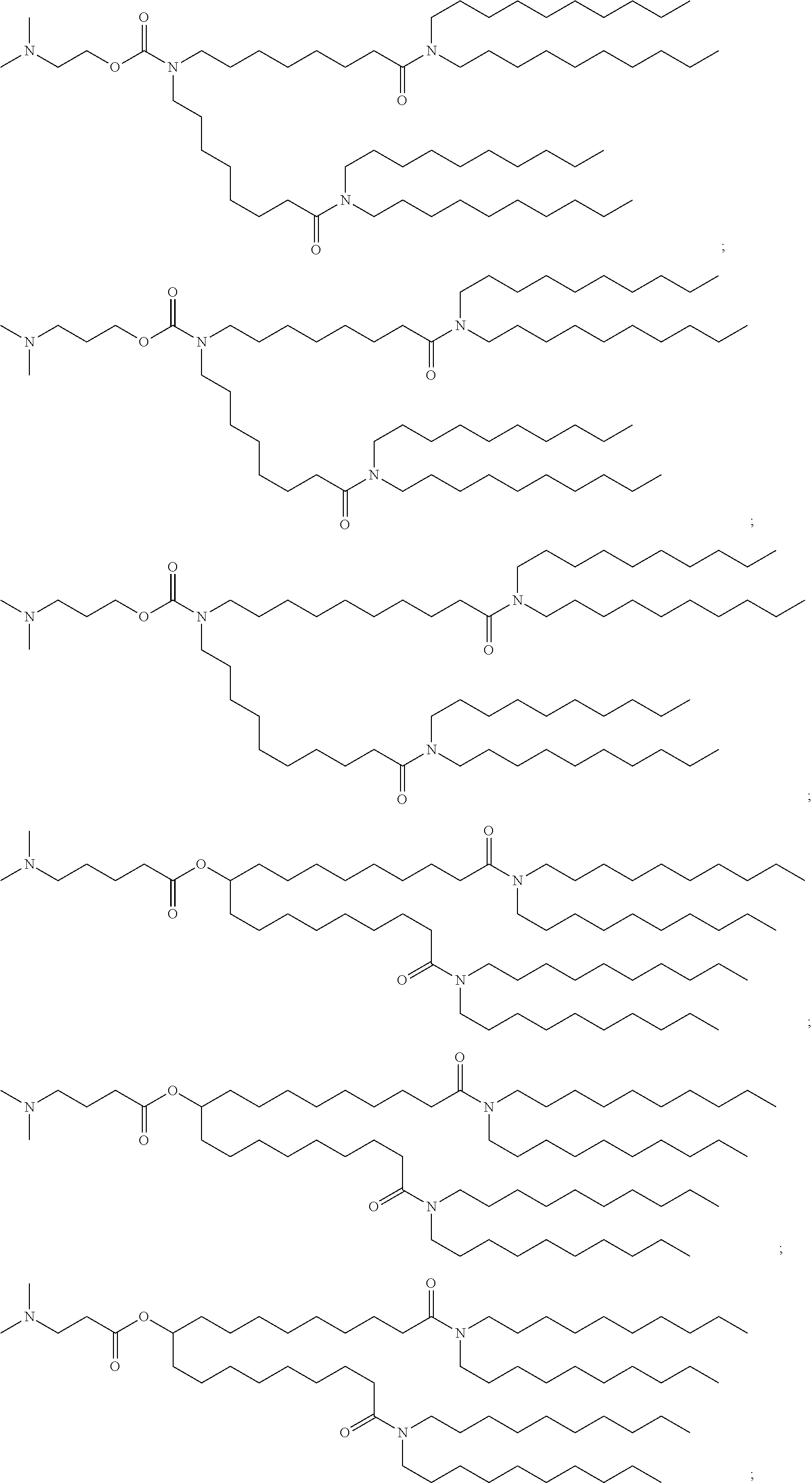 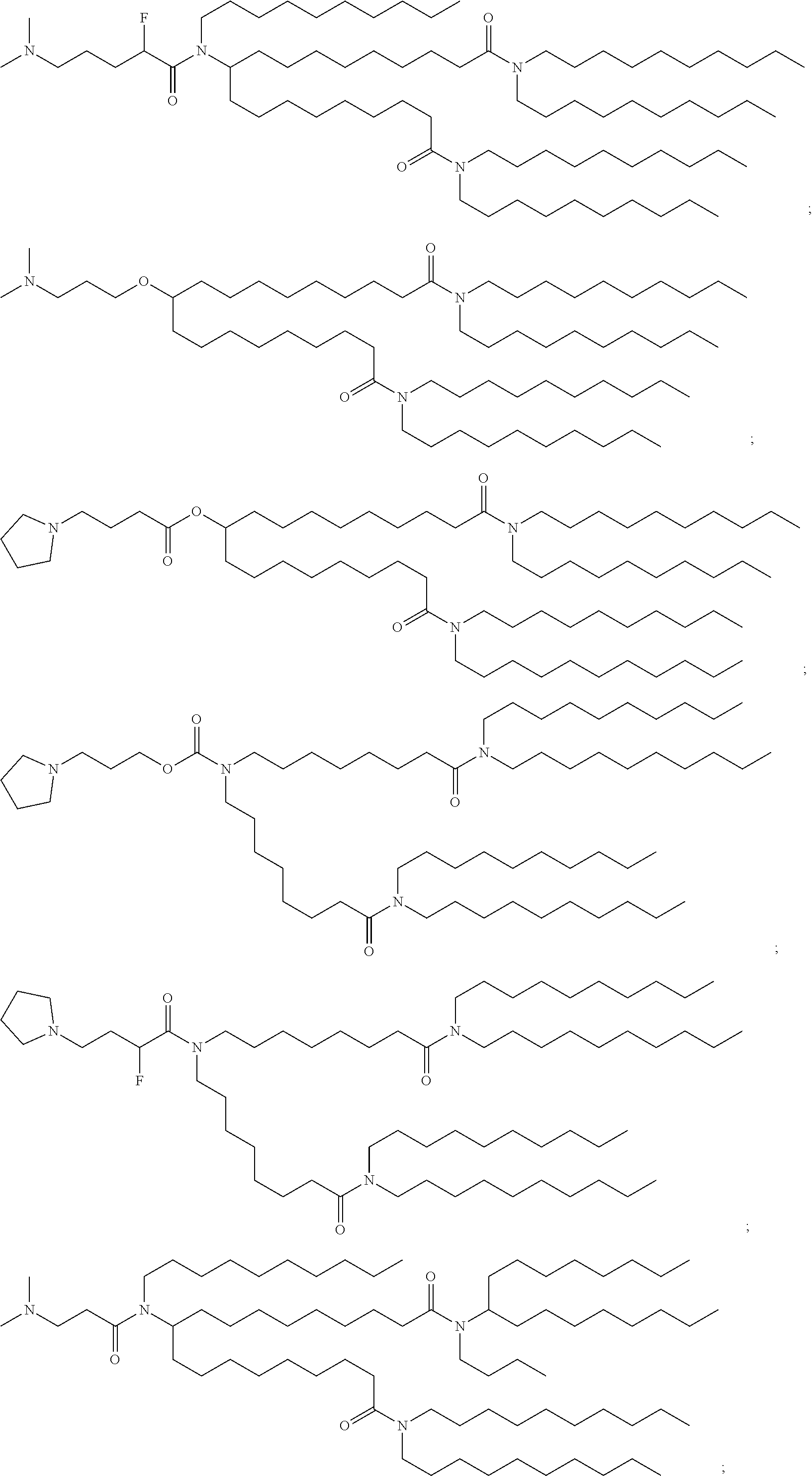 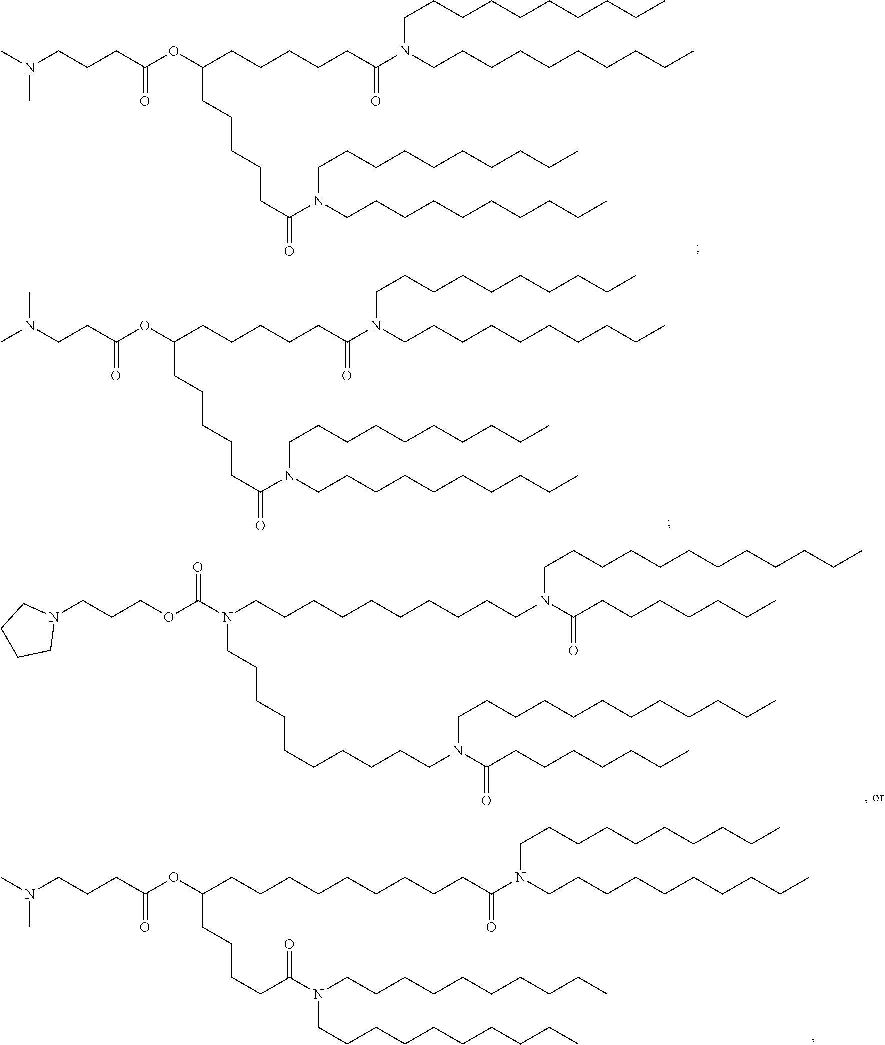
or a pharmaceutically acceptable salt, tautomer, or stereoisomer thereof.

60. A lipid nanoparticle comprising the compound of any one of claim 1 and a therapeutic agent.

61. A composition comprising the compound of claim 1 and a therapeutic agent.

62-75. (canceled)

76. A method for inducing expression of a desired protein in a subject in need thereof, the method comprising administering a therapeutically effective amount of the lipid nanoparticle of claim 60 to the subject, wherein the therapeutic agent comprises a nucleic acid.

77-80. (canceled)
